(19) United States
(12) Patent Application Publication
(10) Pub. No.: US 2009/0012091 A1
(43) Pub. Date: Jan. 8, 2009

(54) Oximide derivatives and their therapeutical application

(75) Inventor: Chang Jun Yu, Pasadena, CA (US)

Correspondence Address: BACON & THOMAS, PLLC, 625 SLATERS LANE, FOURTH FLOOR, ALEXANDRIA, VA, 22314-1176, US

(73) Assignee: KinaGen, Inc., Monrovia, CA (US)

(21) Appl. No.: 12/213,627

(22) Filed: Jun. 23, 2008

Related U.S. Application Data
(60) Provisional application No. 60/958,020, filed on Jul. 2, 2007.

(51) Int. Cl. A61K 31/53; C07D 213/62; A61K 31/44; C07D 239/32; A61P 3/00; A61P 9/00; A61P 35/04; A61P 29/00; A61P 7/00; A61K 31/505; C07D 251/12

(52) U.S. Cl. 514/241; 546/298; 514/350; 544/319; 514/256; 544/219

(57) ABSTRACT

The present invention relates to a compound represented as the following Formula (I) and a pharmaceutical composition thereof

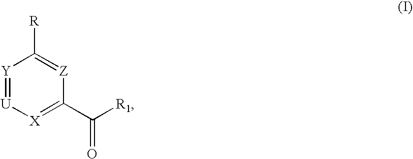

wherein all substituents are as defined in the specification; and also relates to a method for treating or lessening the severity of a disease or a condition, comprising administering said compound or said pharmaceutical composition.

### CROSS REFERENCES TO THE RELATED APPLICATIONS

This application claims the benefit of U.S. Provisional Application No. 60/958,020, filed Jul. 2, 2007.

### BACKGROUND OF THE INVENTION

1. Field of the Invention

The present invention relates generally to the use of compounds to treat a variety of disorders, diseases and pathologic conditions, and more specifically to the use of oximide compounds for modulating protein kinases and for treating Raf kinase-mediated diseases.

2. Description of the Related Art

The kinases may be categorized into families by the substrates in the phosphorylate (e.g., protein-tyrosine, protein-serine/threonine, lipids, etc.). Tyrosine phosphorylation is a central event in the regulation of a variety of biological processes such as cell proliferation, migration, differentiation and survival. Several families of receptor and non-receptor tyrosine kinases control these events by catalyzing the transfer of phosphate from ATP to a tyrosine residue of specific cell protein targets.

Cancer results from the deregulation of the normal processes that control cell division, differentiation and apoptotic cell death. Protein kinases play a critical role in this regulatory process. Such kinases includes, but not limit to, abi, ATK, bcr-abi, Bik, Brk, Btk, c-kit, c-met, c-src, CDK1, CDK2, CDK4, CDK6, cRafi, CSF1R, CSK, EGFR, ErbB2, ErbB3, ErbB4, ERK, Fak, fes, FGFR1, FGFR2, FGFR3, FGFR4, FGFR5, Fgr, FLK4, fit-i, Fps, Frk, Fyn, Hck, IGF-1R, INS-R, Jak, KDR, Lck, Lyn, MEK, p38, PDGFR, P1K, PKC, PYK2, ros, tie, TRK, Yes and Zap70. Protein kinases play a critical role in many other diseases. Those diseases include autoimmune diseases, inflammatory diseases (such. as psoriasis), bone diseases (such as osteoporosis), central nervous system disorders (such as Alzheimer's), metabolic disorders (such as diabetes), neurological and neurodegenerative diseases, cardiovascular diseases, allergies and asthma, and hormone-related diseases and infectious diseases (such as viral and fungal infections). In addition, endothelial cell specific receptor PTKs, such as VEGF-2 and Tie-2, mediate the angiogenic process and are involved in supporting the progression of cancers and other diseases involving uncontrolled vascularization. Accordingly, there has been a substantial effort in medicinal chemistry to find protein kinase inhibitors that are effective as therapeutic agents.

One of the most commonly studied pathways involving kinase regulation is cellular signalling from receptors at the cell surface to the nucleus. One example of this pathway includes a cascade of kinases in which members of the Growth Factor receptor Tyrosine Kinases (such as EGF-R, PDGF-R, VEGF-R, IGF1-R, the Insulin receptor) deliver signals through phosphorylation to other kinases such as Src Tyrosine kinase, and the Raf, Mek and Erk serine/threonine kinase families. Each of these kinases is represented by several family members which play related, but functionally distinct roles. The loss of regulation of the growth factor signaling pathway is a frequent occurrence in cancer as well as other disease states.

Raf protein kinases are key components of signal transduction pathways by which specific extracellular stimuli elicit precise cellular responses in mammalian cells. Activated cell surface receptors activate ras/rap proteins at the inner aspect of the plasma membrane which in turn recruit and activate Raf proteins. Activated Raf proteins phosphorylate and activate the intracellular protein kinases MEK1 and MEK2. In turn, activated MEKs catalyse phosphorylation and activation of p42/p44 mitogen-activated protein kinase (MAPK). A variety of cytoplasmic and nuclear substrates of activated MAPK are known which directly or indirectly contribute to the cellular response to environmental change. Three distinct genes have been identified in mammals that encode Raf proteins; A-Raf, B-Raf and C-Raf (also known as Raf-1) and isoformic variants that result from differential splicing of mRNA are known.

Inhibitors of Raf kinases have been suggested for use in disruption of tumor cell growth and hence in the treatment of cancers, e.g. histiocytic lymphoma, lung adenocarcinoma, small cell lung cancer and pancreatic and breast carcinoma; and also in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth.

There has been increasing interest in recent years in the development of protein kinase inhibitors, particularly Raf inhibitors, as therapeutic agents for the treatment of diseases/conditions involving protein kinase-mediated events. We have now found a group of novel compounds that are inhibitors of Raf kinases, in particular inhibitors of B-Raf kinase.

### SUMMARY OF THE INVENTION

Accordingly, it is an objective of the present invention to provide an antitumor agent comprising a heteroaryl alkoxyamide derivative as described in Formula (I), pharmaceutically-acceptable formulations thereof, methods for making novel compounds and methods and compositions for using the compounds. The compounds and compositions comprising the compounds in Formula (I) have utility in treatment of a variety of diseases.

The combination therapy described herein may be provided by the preparation of the derivatives of Formula (I) and the other therapeutic agent as separate pharmaceutical formulations followed by the administration thereof to a patient simultaneously, semi-simultaneously, separately or over regular intervals.

In certain other embodiments, the invention provides pharmaceutical compositions comprising an inventive compound, wherein the compound is present in an amount effective to inhibit Raf activity. In certain other embodiments, the invention provides pharmaceutical compositions comprising an inventive compound and optionally further comprising an additional therapeutic agent. In yet other embodiments, the additional therapeutic agent is an agent for the treatment of cancer.

In yet another aspect, the present invention provides methods for inhibiting kinase activity (e.g., Raf) activity in a patient or a biological sample, comprising administering to said patient, or contacting said biological sample with an effective inhibitory amount of a compound of the invention. In still another aspect, the present invention provides methods for treating any disorder involving Raf activity, comprising administering to a subject in need thereof a therapeutically effective amount of a compound of the invention.

### DETAILED DESCRIPTION OF THE INVENTION

The present invention is related to a compound represented Formula (I)

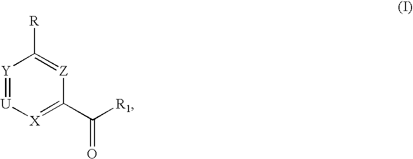

wherein

X represents N or CRₓ; Y represents N or CR_{y}; Z represents N or CR_{z}; U represents N or CRᵤ; wherein Rₓ, R_{y}, R_{z} and Rᵤ independently represent hydrogen, halogen, alkyl, alkoxy, alkylthio, amino, alkylamino or sulfoalkyl, optionally represent Formula (II) if R is not Formula (II); among X, Y, Z and U, at least one of them is N;

R represents
1) hydrogen, halogen, alkyl, alkoxy, alkylthio, amino, alkylamino or sulfoalkyl; or
2) Formula (II):

-L₁-Ar₁-L₂-Ar₂ (II),
wherein
   L₁ and L₂ independently represent NR₂, NR₂CONR₂, NR₂CSR₂, NR₂CO, S, SO, SO₂, O or CONR₃, or optionally represent cycloalkyl and heterocycloalkyl to link Ar₁ and Ar₂; wherein R₂ and R₃ independently represent hydrogen or C₁-C₄ alkyl;
   Ar₁ and Ar₂ independently represent an aryl or heteroaryl, each of which is substituted with from 0 to 4 substituents independently chosen from:
      (1) halogen, hydroxy, amino, cyano, —COOH, —SO₂NH₂, oxo, nitro or alkoxycarbonyl; or
      (2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₆alkyl)amino, C₁-C₆ alkylsulfonyl, mono- or di-(C₁-C₆alkyl) sulfonamido, mono- or di-(C₁-C₆alkyl)aminocarbonyl, phenylC₀-C₄alkyl or (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy or C₁-C₄haloalkyl;

R1 represents
1) OR₄
2) NR₄R₅, or
3) Formula (III)

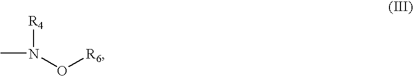

wherein R₄, R₅ and R₆ independently represent hydrogen, C₁-C₁₀ alkyl, C₁-C₁₀alkoxy, C₃-C₁₀ cycloalkyl, C₂-C₁₀ alkenyl, C₂-C₁₀ alkynyl, C₂-C₁₀ alkanoyl, C₁-C₁₀ haloalkyl, C₁-C₁₀ haloalkoxy, mono- or di-(C₁-C₁₀alkyl)amino, C₁-C₁₀ alkylsulfonyl, mono- or di-(C₁-C₁₀alkyl) sulfonamido, mono- or di-(C₁-C₆alkyl)aminocarbonyl, phenylC₀-C₆alkyl or (4- to 7-membered heterocycle)C₀-C₆alkyl, each of which is substituted with from 0 to 4 secondary substituents independently chosen from halogen, hydroxy, cyano, oxo, amino, imino, C₁-C₄alkyl, C₁-C₄alkoxy or C₁-C₄haloalkyl; and

only when X is N and R is Formula (II), R1 represents Formula (III).

The following definitions refer to the various terms used above and throughout the disclosure.

When used herein, the term “halo” or “halogen” includes, unless otherwise defined, fluorine, chlorine, bromine or iodine.

When used herein, the term “alkyl” includes, unless otherwise defined, a monovalent alkane (hydrocarbon) derived radical containing from 1 to 12 carbon atoms. Alkyl groups may be substituted at any available point of attachment. An alkyl group substituted with another alkyl group is also referred to as a “branched alkyl group”. Exemplary alkyl groups include methyl, ethyl, propyl, isopropyl, n-butyl, t-butyl, isobutyl, pentyl, hexyl, isohexyl, heptyl, dimethylpentyl, octyl, 2,2,4-trimethylpentyl, nonyl, decyl, undecyl, dodecyl, and the like. Exemplary substituents include, but are not limited to, one or more of the following groups: alkyl, aryl, halo (such as F, Cl, Br, I), haloalkyl (such as CCl₃ or CF₃), alkoxy, alkylthio, hydroxy, carboxy (—COOH), alkyloxycarbonyl (—C(O)R), alkylcarbonyloxy (—OCOR), amino (—NH₂), carbamoyl (—NHCOOR— or —OCONHR—), urea (—NHCONHR—) or thiol (—SH). In some preferred embodiments of the present invention, alkyl groups are substituted with, for example, amino, heterocycloalkyl, such as morpholine, piperazine, piperidine, azetidine, hydroxyl, methoxy, or heteroaryl groups such as pyrrolidine.

When used herein, the term “cycloalkyl” includes, unless otherwise defined, fully saturated and partially unsaturated hydrocarbon rings of 3 to 9, preferably 3 to 7 carbon atoms. The examples include cyclopropyl, cyclobutyl, cyclopentyl and cyclohexyl, and the like. Further, a cycloalkyl may be substituted. A substituted cycloalkyl refers to such rings having one, two, or three substituents, selected from the group consisting of halo, alkyl, substituted alkyl, alkenyl, alkynyl, nitro, cyano, oxo (═O), hydroxy, alkoxy, thioalkyl, —CO2H, —C(═O)H, CO2-alkyl, —C(═O)alkyl, keto, ═N—OH, ═N—O-alkyl, aryl, heteroaryl, heterocyclo, —NR′R″, —C(═O)NR′R″, —CO2NR′R″, —C(═O)NR′R″, —NR′CO2R″, —NR′C(═O)R″, —SO2NR′R″, and —NR′SO2R″, wherein each of R′ and R″ are independently selected from hydrogen, alkyl, substituted alkyl, or cycloalkyl, or R′ and R″ together form a heterocyclo or heteroaryl ring.

When used herein, the term “alkenyl” includes, unless otherwise defined, a hydrocarbon radical straight, branched or cyclic containing from 2 to 12 carbon atoms and at least one carbon to carbon double bond. Examples of such groups include the vinyl, allyl, 1-propenyl, isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, and the like. Alkenyl groups may also be substituted at any available point of attachment. Exemplary substituents for alkenyl groups include those listed above for alkyl groups, and especially include C3 to C7 cycloalkyl groups such as cyclopropyl, cyclopentyl and cyclohexyl, which may be further substituted with, for example, amino, oxo, hydroxyl, etc.

When used herein, the term “alkynyl” includes, unless otherwise defined, straight or branched chain alkyne groups, which have one or more unsaturated carbon-carbon bonds, at least one of which is a triple bond. Alkynyl groups include C2-C8 alkynyl, C2-C6 alkynyl and C2-C4 alkynyl groups, which have from 2 to 8, 2 to 6 or 2 to 4 carbon atoms, respectively. Illustrative of the alkynyl group include ethenyl, propenyl, isopropenyl, butenyl, isobutenyl, pentenyl, and hexenyl. Alkynyl groups may also be substituted at any available point of attachment. Exemplary substituents for alkynyl groups include those listed above for alkyl groups such as amino, alkylamino, etc. The numbers in the subscript after the symbol “C” define the number of carbon atoms a particular group can contain.

When used herein, the term “alkoxy” includes, unless otherwise defined, alone or as part of another group denotes an alkyl group as described above bonded through an oxygen linkage (—O—). Preferred alkoxy groups have from 1 to 8 carbon atoms. Examples of such groups include the methoxy, ethoxy, n-propoxy, isopropoxy, n-butoxy, isobutoxy, sec-butoxy, tert-butoxy, n-pentyloxy, isopentyloxy, n-hexyloxy, cyclohexyloxy, n-heptyloxy, n-octyloxy and 2-ethylhexyloxy.

When used herein, the term “alkylthio” includes, unless otherwise defined, an alkyl group as described above attached via a sulfur bridge. Preferred alkoxy and alkylthio groups are those in which an alkyl group is attached via the heteroatom bridge. Preferred alkylthio groups have from 1 to 8 carbon atoms. Examples of such groups include the methylthio, ethylthio, n-propythiol, n-butylthiol, and the like.

When used herein, the term “oxo” refers to, unless otherwise defined, a keto (C═O) group. An oxo group that is a substituent of a nonaromatic carbon atom results in a conversion of —CH2- to —C(═O)—.

When used herein, the term “aryl” refers to, unless otherwise defined, monocyclic or bicyclic aromatic rings, e.g. phenyl, substituted phenyl and the like, as well as groups which are fused, e.g., napthyl, phenanthrenyl and the like. An aryl group thus contains at least one ring having at least 6 atoms, with up to five such rings being present, containing up to 20 atoms therein, with alternating (resonating) double bonds between adjacent carbon atoms or suitable heteroatoms. Aryl groups may optionally be substituted with one or more groups including, but not limited to, halogen, such as I, Br, F or Cl, alkyl, such as methyl, ethyl or propyl, alkoxy, such as methoxy or ethoxy, hydroxy, carboxy, carbamoyl, alkyloxycarbonyl, nitro, alkenyloxy, trifluoromethyl, amino, cycloalkyl, aryl, heteroaryl, cyano, alkyl S(O)m (m=0, 1, 2), or thiol.

When used herein, the term “aromatic” refers to, unless otherwise defined, a cyclically conjugated molecular entity with a stability, due to delocalization, significantly greater than that of a hypothetical localized structure, such as the Kekule structure.

When used herein the term “heterocyclyl” includes, unless otherwise defined, non-aromatic, single and fused, rings suitably containing up to four heteroatoms in each ring, each of which is selected from O, N and S, which rings, may be unsubstituted or substituted by, for example, up to three substituents. Each heterocyclic ring suitably has from 4 to 7, preferably 5 or 6, ring atoms. A fused heterocyclic ring system may include carbocyclic rings and include only one heterocyclic ring. Examples of heterocyclyl groups include pyrrolidine, piperidine, piperazine, morpholine, imidazolidine and pyrazolidine.

When used herein, the term “heteroaryl” includes, unless otherwise defined, mono- and bicyclic heteroaromatic ring systems comprising up to four, preferably 1 or 2, heteroatoms, each selected from O, N and S. Each ring may have from 4 to 7, preferably 5 or 6, ring atoms. A bicyclic heteroaromatic ring system may include a carbocyclic ring. Examples of heteroaryl groups include pyrrole, quinoline, isoquinoline, pyridine, pyrimidine, oxazole, thiazole, thiadiazole, triazole, imidazole and benzimidazole.

Aryl, heterocyclyl and heteroaryl groups may be optionally substituted by preferably up to three substituents. Suitable substituents include halogen, C₁₋₆alkyl, aryl, aryl C₁₋₆alkyl, C₁₋₆alkoxy, C₁₋₆alkoxy C₁₋₆alkyl, halo C₁₋₆alkyl, arylC₁₋₆alkoxy, hydroxy, nitro, cyano, azido, amino, mono- and di-N—C₁₋₆alkylamino, acylamino, arylcarbonylamino, acyloxy, carboxy, carboxy salts, carboxy esters, carbamoyl, mono- and di-N—C₁₋₆alkylcarbamoyl, C₁₋₆alkoxycarbonyl, aryloxycarbonyl, ureido, guanidino, C₁₋₆alkylguanidino, amidino, C₁₋₆alkylamidino, sulphonylamino, aminosulphonyl, C₁₋₆alkylthio, C₁₋₆alkylsulphinyl, C₁₋₆alkylsulphonyl, heterocyclyl, heteroaryl, heterocyclyl C₁₋₆alkyl, hydroxyimino-C₁₋₆alkyl and heteroaryl C₁₋₆alkyl and combinations thereof. Preferably the optional substituent contains a solubilising group; suitable solubilising moieties will be apparent to those skilled in the art and include hydroxy and amine groups. Even more preferably the optional substituent include heterocyclyl, amino, mono- or di-C₁₋₆alkylamino, amide and hydroxy, or any combination thereof.

When used herein, the term “amino” includes, unless otherwise defined, —NH2. An “amino” may optionally be substituted with one or two substituents, which may be the same or different, such as alkyl, aryl, arylalkyl, alkenyl, alkynyl, heteroaryl, heteroarylalkyl, cycloheteroalkyl, cycloheteroalkylalkyl, cycloalkyl, cycloalkylalkyl, haloalkyl, hydroxyalkyl, alkoxyalkyl, thioalkyl, carbonyl or carboxyl. These substituents may be further substituted with a carboxylic acid, any of the alkyl or aryl substituents set out herein. In some embodiments, the amino groups are substituted with carboxyl or carbonyl to form N-acyl or N-carbamoyl derivatives.

When used herein, the term “alkylsulfonyl” includes, unless otherwise defined, groups of the formula (SO2)-alkyl, in which the sulfur atom is the point of attachment. Preferably, alkylsulfonyl groups include C1-C6 alkylsulfonyl groups, which have from 1 to 6 carbon atoms. Methylsulfonyl is one representative alkylsulfonyl group.

When used herein, the term “heteroatom” includes, unless otherwise defined, any atom other than carbon, for example, N, O, or S.

When used herein, the term “optionally substituted” refers, unless otherwise defined, that the aryl or heterocyclyl or other group may be substituted at one or more substitutable positions by one or more groups independently selected from alkyl (preferably lower alkyl), alkoxy (preferably lower alkoxy), nitro, monoalkylamino (preferably with one to six carbons), dialkylamino (preferably with one to six carbons), cyano, halo, haloalkyl (preferably trifluoromethyl), alkanoyl, aminocarbonyl, monoalkylaminocarbonyl, dialkylaminocarbonyl, alkyl amido (preferably lower alkyl amido), alkoxyalkyl (preferably a lower alkoxy or lower alkyl), alkoxycarbonyl (preferably a lower alkoxycarbonyl), alkylcarbonyloxy (preferably a lower alkylcarbonyloxy) or aryl (preferably phenyl), said aryl being optionally substituted by halo, lower alkyl or lower alkoxy groups. Optional substitution is also indicated by the phrase “substituted with from 0 to X substituents,” where X is the maximum number of possible substituents. Certain optionally substituted groups are substituted with from 0 to 2, 3 or 4 independently selected substituents.

A dash (“-”) that is not between two letters or symbols is used to indicate a point of t attachment for a substituent. For example, —CONH2 is attached through the carbon atom.

When used herein, the term “kinase” refers to, unless otherwise defined, any enzyme that catalyzes the addition of phosphate groups to a protein residue; for example, serine and threonine kineses catalyze the addition of phosphate groups to serine and threonine residues.

When used herein, the term “Raf kinase” refers to, unless otherwise defined, a 74 kDa serine/threonine kinase and the related homologs or analogs belonging to the mammalian family of Raf kineses, including, for example, A-, B- and C-Raf.

When used herein, the term “therapeutically effective amount” refers to, unless otherwise defined, the amount of the compound or pharmaceutical composition that will elicit the biological or medical response of a tissue, system, animal or human that is being sought by the researcher, veterinarian, medical doctor or other clinician, e.g., restoration or maintenance of vasculostasis or prevention of the compromise or loss or vasculostasis; reduction of tumor burden; reduction of morbidity and/or mortality.

When used herein, the term “pharmaceutically acceptable” refers to the fact that the carrier, diluent or excipient must be compatible with the other ingredients of the formulation and not deleterious to the recipient thereof.

When used herein, the terms “administration of a compound” or “administering a compound” refer to, unless otherwise defined, the act of providing a compound of the invention or pharmaceutical composition to the subject in need of treatment.

When used herein, the term “protected” refers, unless otherwise defined, that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, John Wiley & Sons, New York (1999).

When used herein, the term “pharmaceutically acceptable salt” refers to, unless otherwise defined, an acid or base salt that is suitable for use in contact with the tissues of human beings or animals without excessive toxicity or carcinogenicity, and preferably without irritation, allergic response, or other problem or complication, and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof means that a metabolite or residue thereof is also an inhibitor of a Raf kinase.

Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(alkyl)₄ salts. This invention also envisions the quaternization of any basic nitrogen-containing groups of the compounds disclosed herein. Water or oil-soluble or dispersable products may be obtained by such quaternization.

Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Preferred compounds in Formula (I) are list below, wherein the substitute may be the specific ones as defined here or may be one or multiple substitutes as defined above:

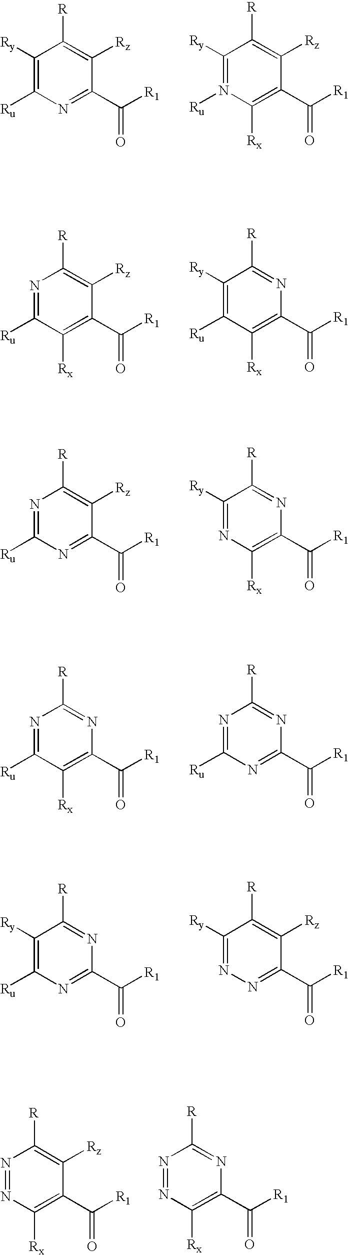

The examples of the preferred R6 group of Formula (III) are list below, wherein the substitute may be the specific ones as defined here or may be one or multiple substitutes as defined above:

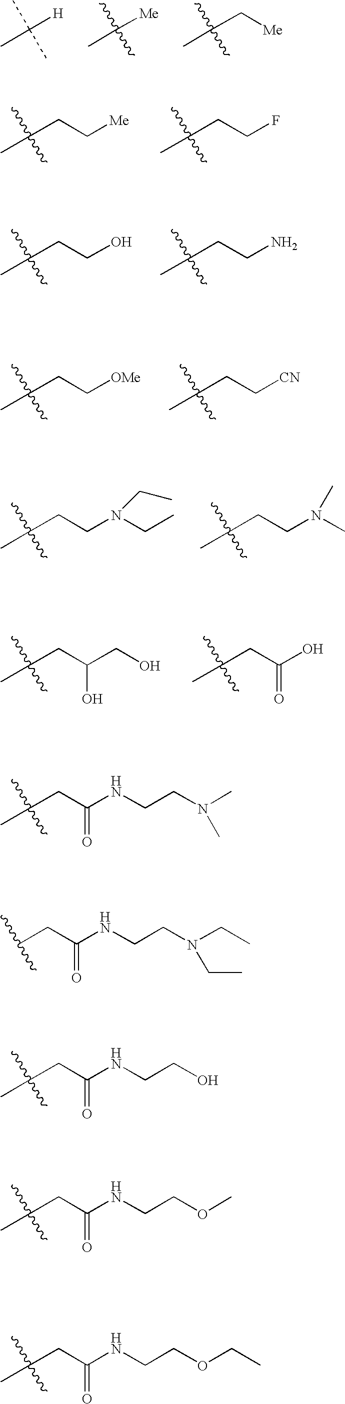

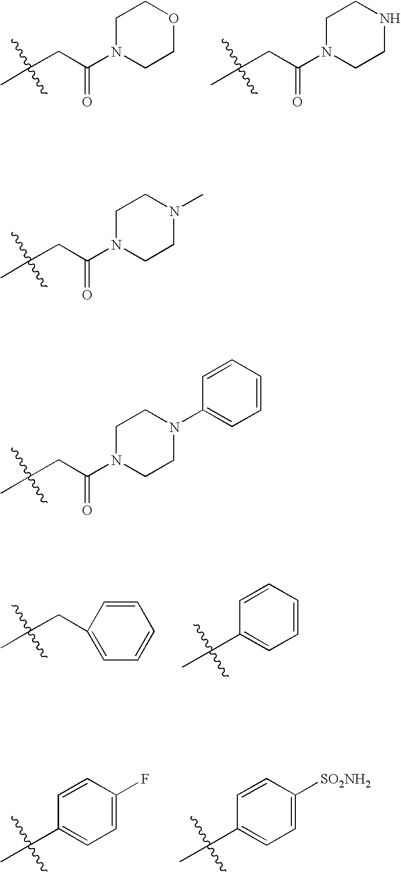

Preferred R groups of Formula (II) are list below, wherein the substitute may be the specific ones as defined here or may be one or multiple substitutes as defined above:

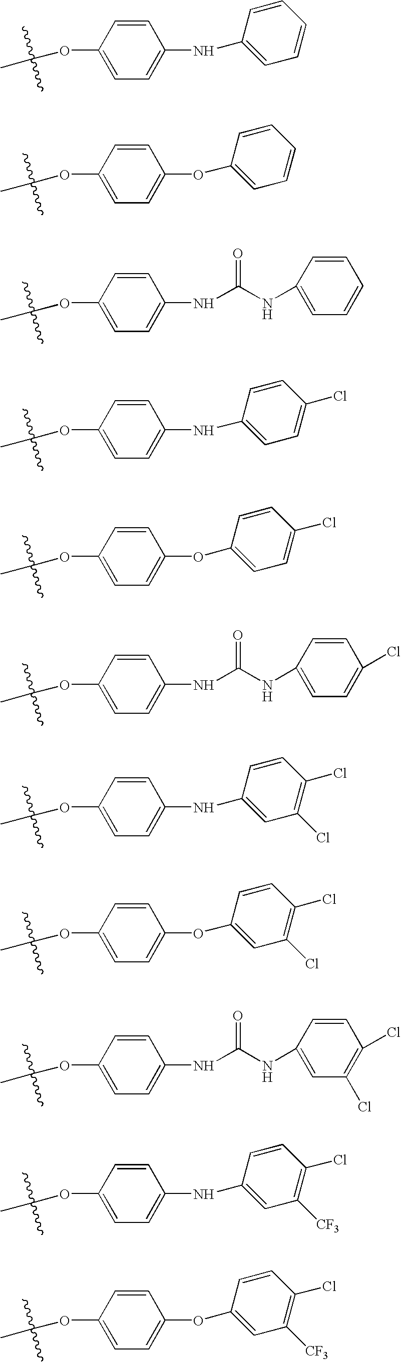

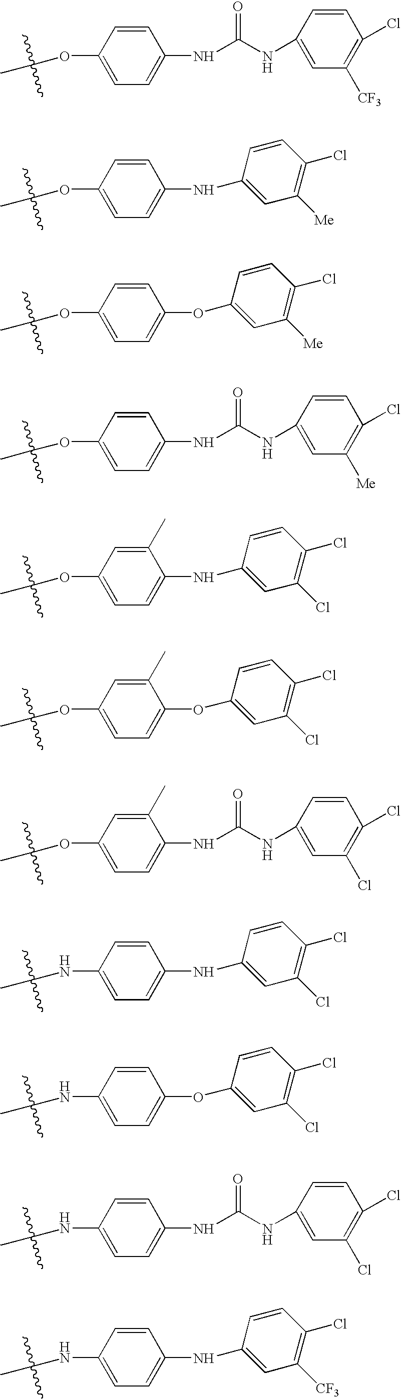

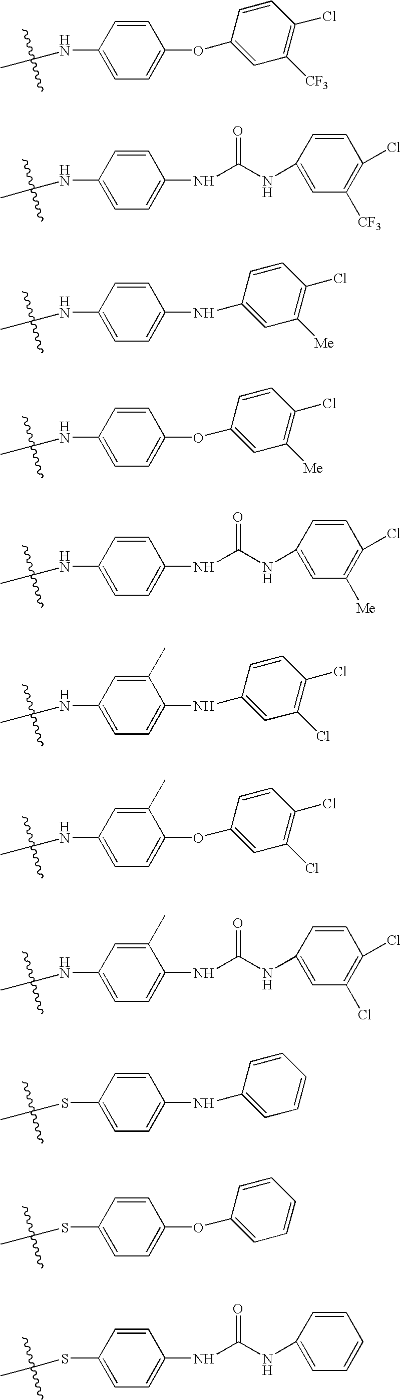

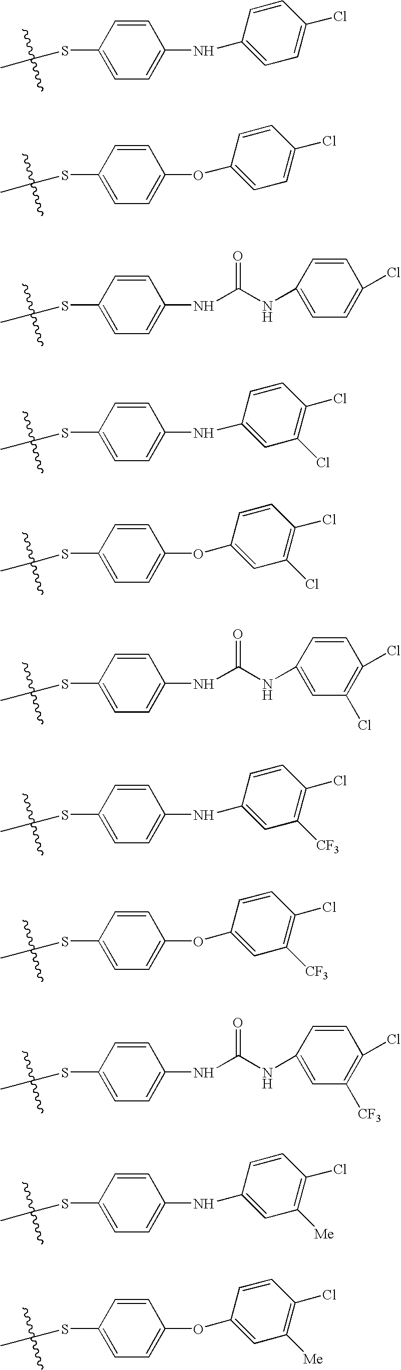

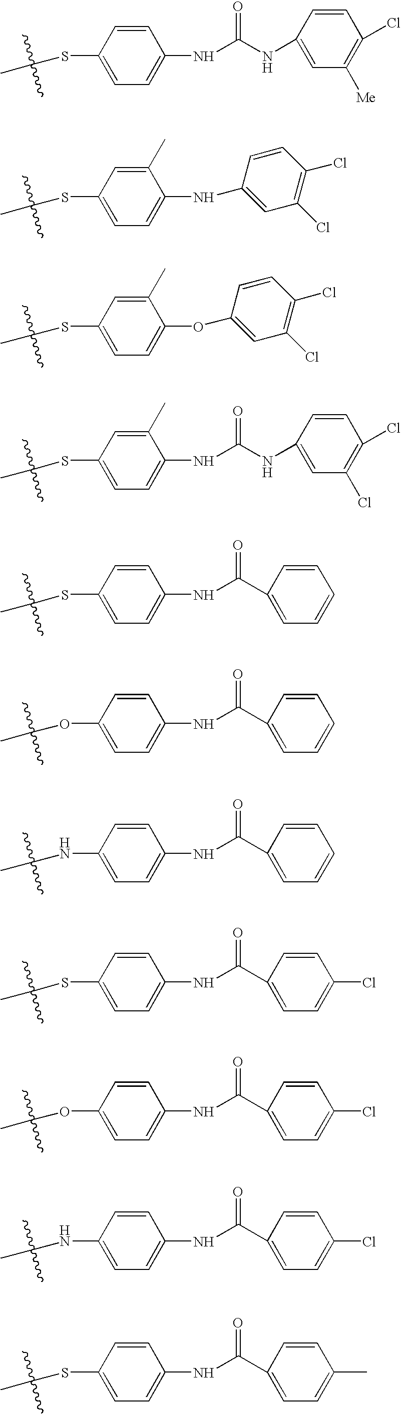

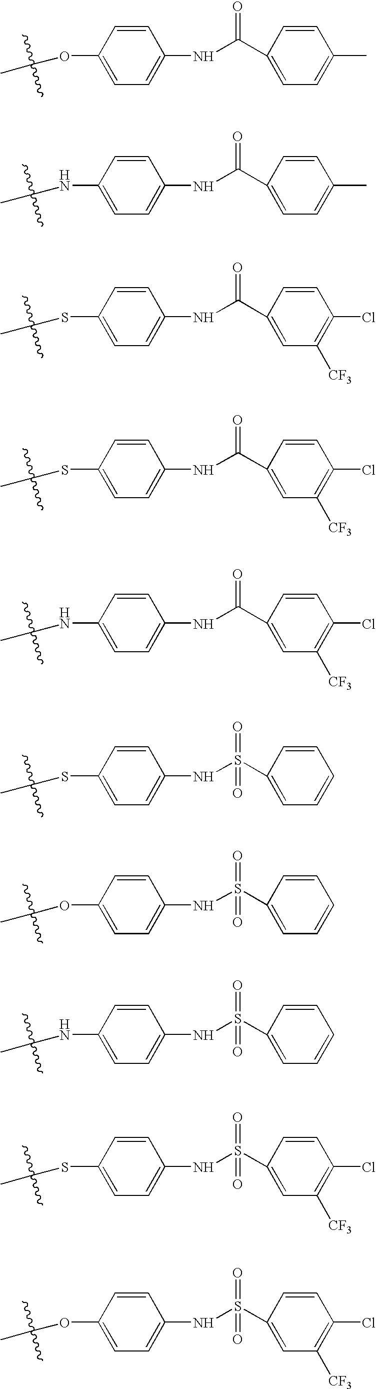

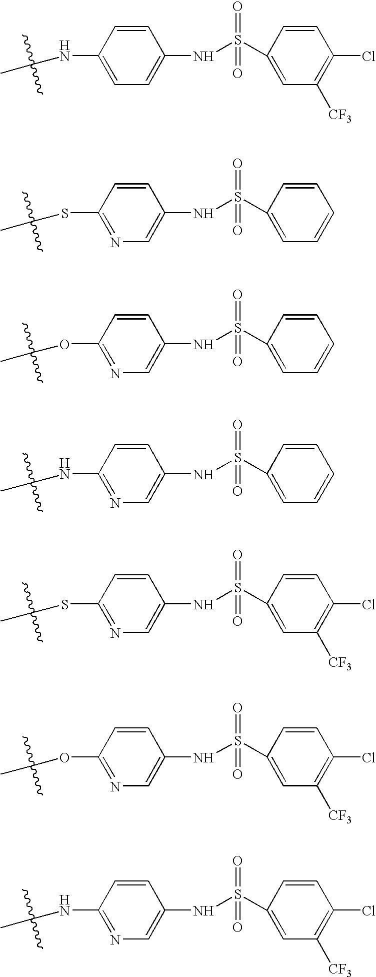

Preferred Ar1 groups of Formula (I) are list below, wherein the substitute may be the specific ones as defined here or may be one or multiple substitutes as defined above:

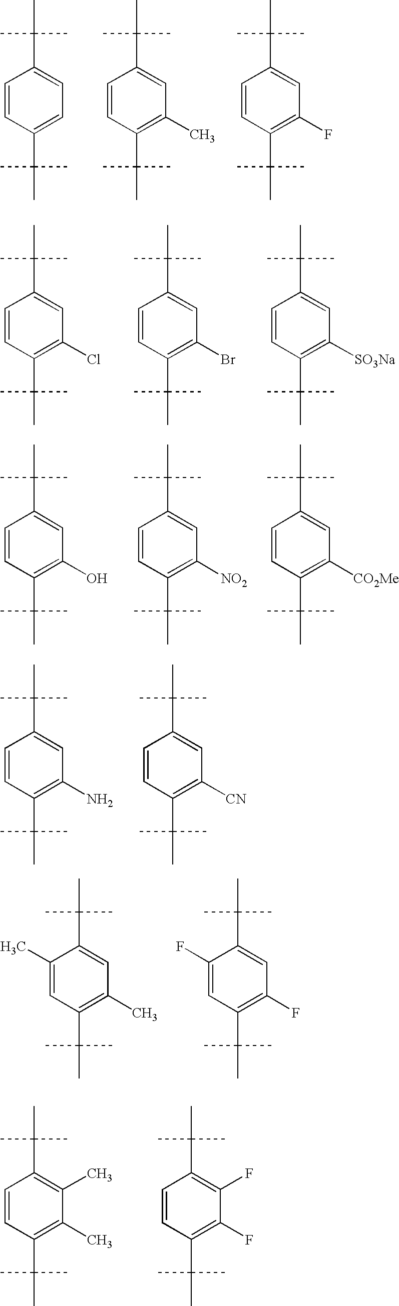

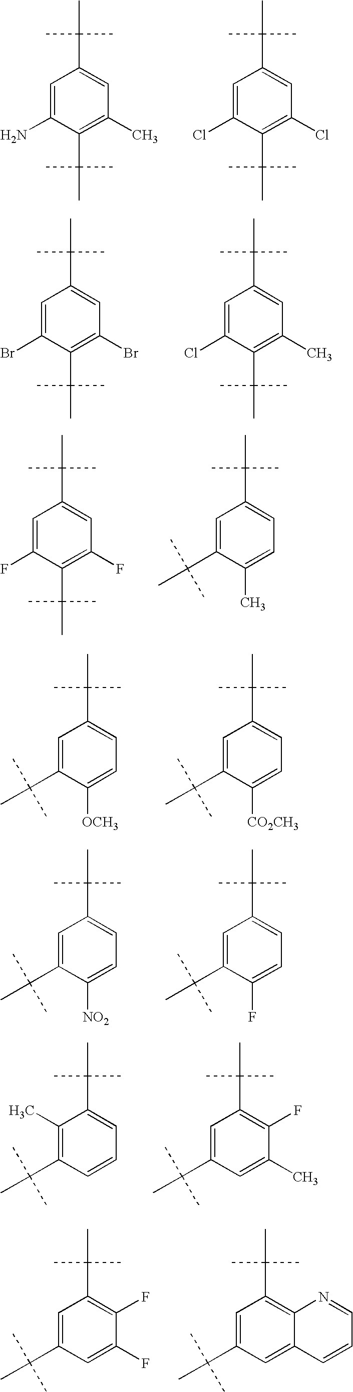

Preferred Ar2 groups of Formula (I) are list below, wherein the substitute may be the specific ones as defined here or may be one or multiple substitutes as defined above:

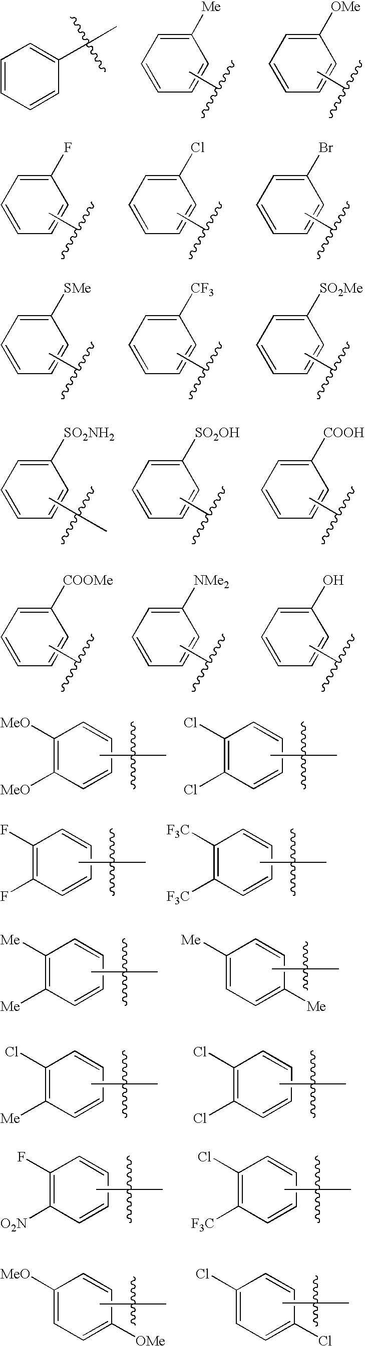

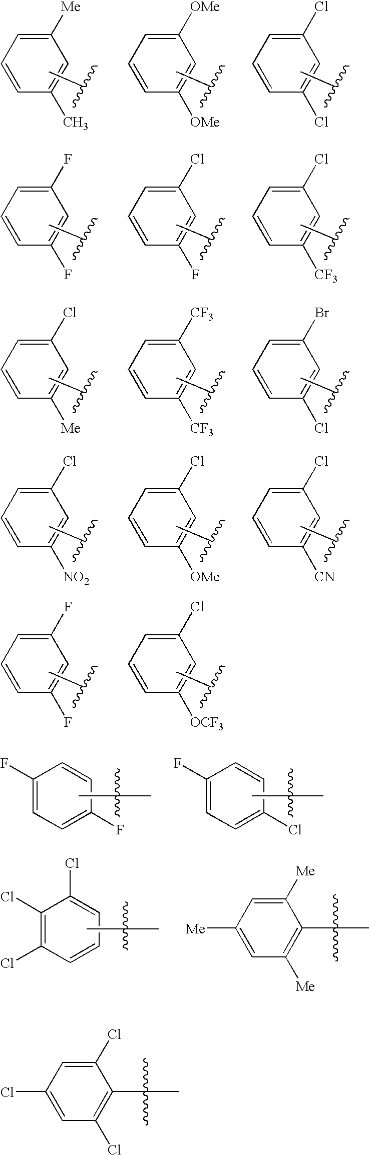

Ar1 and Ar2 are preferably an optionally substituted phenyl. Preferred substituents for the group Ar1 and Ar2 include halo, hydroxy, hydroxy C₁₋₆alkyl e.g. hydroxymethyl, hydroxyimino-C₁₋₆alkyl and C₁₋₆alkoxy e.g. methoxy. More preferred are halo and hydroxy. When Ar2 is phenyl, the substituents are preferably present in the 3-position or the 3,4-positions.

Preferably for the Formula (I), X represents N or CRₓ; Y represents N or CR_{y}; Z represents N or CR_{z; U represents N or CR}ᵤ; wherein Rₓ, R_{y}, R_{z} and Rᵤ independently represent hydrogen, halogen, alkyl, alkoxy, alkylthio, amino, alkylamino or sulfoalkyl, optionally represent Formula (II) if R is not selected from Formula (II); among X, Y, Z and U, at least one of them is N;

R represents
1) hydrogen, halogen, alkyl, alkoxy, alkylthio, amino, alkylamino or sulfoalkyl; or
2) Formula (II):

-L₁-Ar₁-L₂-Ar₂ (II),
wherein
L₁ and L₂ independently represent NR₂, NR₂CONR₂, NR₂CSR₂, NR₂CO, SR₃, SO, SO₂ or CONR₃, or optionally represent cycloalkyl or heterocycloalkyl to link Ar₁ and Ar₂; wherein R₂ and R₃ represents hydrogen or C₁-C₄ alkyl;
Ar₁ and Ar₂ independently represent an aryl or heteroaryl, each of which is substituted with from 0 to 4 substituents independently chosen from:
   (1) halogen, hydroxy, amino, cyano, —COOH, —SO₂NH₂, oxo, nitro or alkoxycarbonyl; or
   (2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₆alkyl)amino, C₁-C₆ alkylsulfonyl, mono- or di-(C₁-C₆alkyl) sulfonamido or mono- or di-(C₁-C₆alkyl)aminocarbonyl, phenylC₀-C₄alkyl or (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy or C₁-C₄haloalkyl;

R₁ represents
1) NR₄R₅, or
2) Formula (III)

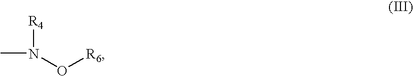

wherein R₄, R₅ and R₆ independently represent hydrogen, C₁-C₁₀ alkyl, C₁-C₁₀alkoxy, C₃-C₁₀ cycloalkyl, C₂-C₁₀ alkenyl, C₂-C₁₀ alkynyl, C₂-C₁₀ alkanoyl, C₁-C₁₀ haloalkyl, C₁-C₁₀ haloalkoxy, mono- or di-(C₁-C₁₀alkyl)amino, C₁-C₁₀ alkylsulfonyl, mono- or di-(C₁-C₁₀alkyl) sulfonamido, mono- or di-(C₁-C₆alkyl)aminocarbonyl, phenylC₀-C₆alkyl or (4- to 7-membered heterocycle)C₀-C₆alkyl, each of which is substituted with from 0 to 4 secondary substituents independently chosen from halogen, hydroxy, cyano, oxo, amino, imino, C₁-C₄alkyl, C₁-C₄alkoxy or C₁-C₄haloalkyl.

More preferably for the Formula (I), X represents N or CRₓ; Y represents N or CR_{y}; Z represents N or CR_{z}; U represents N or CRᵤ; wherein Rₓ, R_{y}, R_{z} and Rᵤ independently represent hydrogen, halogen, alkyl, alkoxy, alkylthio, amino, alkylamino or sulfoalkyl, optionally represent Formula (II) if R is not selected from Formula (II); among X, Y, Z and U, at least one of them is N;

R represents
1) hydrogen, halogen, alkyl, alkoxy, alkylthio, amino, alkylamino or sulfoalkyl; or
2) Formula (II):

-L1-Ar1-L2-Ar2 (II),

wherein
L₁ and L₂ independently represent NR₂, NR₂CONR₂, NR₂CSR₂, NR₂CO, SR₃, SO, SO₂ or CONR₃, or optionally represent cycloalkyl or heterocycloalkyl to link Arm and Ar₂; wherein R₂ and R₃ independently represent hydrogen or C₁-C₄ alkyl;
Ar₁ and Ar₂ independently represent an aryl or heteroaryl, each of which is substituted with from 0 to 4 substituents independently chosen from:
   (1) halogen, hydroxy, amino, cyano, —COOH, —SO₂NH₂, oxo, nitro or alkoxycarbonyl; or
   (2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₆alkyl)amino, C₁-C₆ alkylsulfonyl, mono- or di-(C₁-C₆alkyl) sulfonamido, mono- or di-(C₁-C₆alkyl)aminocarbonyl, phenylC₀-C₄alkyl or (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy or C₁-C₄haloalkyl;

R₁ represents
1) NR₄R₅, or
2) Formula (III)

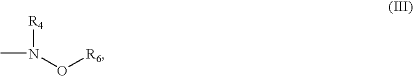

wherein R₄, R₅ and R₆ independently represent hydrogen, C₁-C₆ alkyl, C₁-C₆alkoxy, C₃-C₆ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₁₀alkyl)amino, C₁-C₆ alkylsulfonyl, mono- or di-(C₁-C₆alkyl) sulfonamido, mono- or di-(C₁-C₆alkyl)aminocarbonyl, phenylC₀-C₆alkyl or (4- to 7-membered heterocycle)C₀-C₆alkyl, each of which is substituted with from 0 to 4 secondary substituents independently chosen from halogen, hydroxy, cyano, oxo, amino, imino, C₁-C₄alkyl, C₁-C₄alkoxy or C₁-C₄haloalkyl;

only when X is N and R is Formula (II), R1 represents Formula (III).

Most preferably, X represents N or CRₓ; Y represents N or CR_{y}; Z represents N or CR_{z}; U represents N or CRᵤ; wherein Rₓ, R_{y}, R_{z} and Rᵤ independently represent hydrogen, halogen, alkyl, alkoxy, alkylthio, amino, alkylamino or sulfoalkyl;

R represents
Formula (II):

-L1-Ar1-L2-Ar2 (II),

wherein
L₁ and L₂ independently represent NR₂, NR₂CONR₂, NR₂CSR₂, NR₂CO, SR₃, SO, SO₂ or CONR₃, or optionally represent cycloalkyl or heterocycloalkyl to link Ar₁ and Ar₂; wherein R₂ and R₃ independently represent hydrogen or C₁-C₄ alkyl;
Ar₁ and Ar₂ independently represent an aryl or heteroaryl, each of which is substituted with from 0 to 4 substituents independently chosen from:
   (1) halogen, hydroxy, amino, cyano, —COOH, —SO₂NH₂, oxo, nitro or alkoxycarbonyl; or
   (2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₆alkyl)amino, C₁-C₆ alkylsulfonyl, mono- or di-(C₁-C₆alkyl) sulfonamido, mono- or di-(C , —C₆alkyl)aminocarbonyl, phenylC₀-C₄alkyl or (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy or C₁-C₄haloalkyl;

R₁ represents
1) NR₄R₅, or
2) Formula (III)

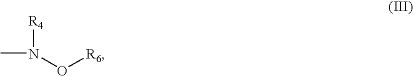

wherein R₄, R₅ and R₆ independently represent hydrogen, C₁-C₁₀ alkyl, C₁-C₁₀alkoxy, C₃-C₁₀ cycloalkyl, C₂-C₁₀ alkenyl, C₂-C₁₀ alkynyl, C₂-C₁₀ alkanoyl, C₁-C₁₀ haloalkyl, C₁-C₁₀ haloalkoxy, mono- or di-(C₁-C₁₀alkyl)amino, C₁-C₁₀ alkylsulfonyl, mono- or di-(C₁-C₁₀alkyl) sulfonamido, mono- or di-(C₁-C₆alkyl)aminocarbonyl, phenylC₀-C₆alkyl or (4- to 7-membered heterocycle)C₀-C₆alkyl, each of which is substituted with from 0 to 4 secondary substituents independently chosen from halogen, hydroxy, cyano, oxo, amino, imino, C₁-C₄alkyl, C₁-C₄alkoxy or C₁-C₄haloalkyl;

only when X is N and R is Formula (II), R1 represents Formula (III).

According to one embodiment, the present invention relates to a compound of Formula (I), wherein Ar1 is phenyl

According to another embodiment, the present invention relates to a compound of Formula (I), wherein Ar2 is 2-methyl-6-chloro-phenyl.

According to another embodiment, the present invention relates to a compound of Formula (I), wherein Ar2 is 2,6-dichlorophenyl.

According to another embodiment, the present invention relates to a compound of Formula (I), wherein Ar2 is 2,6-dimethylphenyl.

According to another embodiment, the present invention relates to a compound of Formula (I), wherein Ar2 is 3-trifluoromethyl-4-chlorophenyl.

According to another embodiment, the present invention relates to a compound of Formula (I), wherein R4 is hydrogen.

According to another embodiment, the present invention relates to a compound of Formula (I), wherein R2 is hydrogen.

According to another embodiment, the present invention relates to a compound of Formula (I), wherein R6 is methyl.

According to another embodiment, the present invention relates to a compound of Formula (I), wherein R6 is ethyl.

According to another embodiment, the present invention relates to a compound of Formula (I), wherein L1 is oxygen.

According to another embodiment, the present invention relates to a compound of Formula (I), wherein L1 is S.

According to another embodiment, the present invention relates to a compound of Formula (I), wherein L2 is CO.

According to another embodiment, the present invention relates to a compound of Formula (I), wherein L2 is NHCO.

According to another embodiment, the present invention relates to a compound of Formula (I), wherein L2 is CONH.

According to another embodiment, the present invention relates to a compound of Formula (I), wherein L2 is NHCONH.

According to another embodiment, the present invention relates to a compound of Formula (I), wherein L2 is NHCSNH.

According to another embodiment, the present invention relates to a compound of Formula (I), wherein L2 is NH.

According to another embodiment, the present invention relates to a compound of Formula (I), wherein L2 is S.

According to another embodiment, the present invention relates to a compound of Formula (I), wherein L2 is SO.

According to another embodiment, the present invention relates to a compound of Formula (I), wherein L2 is SO2.

According to another embodiment, the present invention relates to a compound of Formula (I), wherein L2 is NHSO2.

Examples of specific compounds of the present invention are those compounds defined in the following:

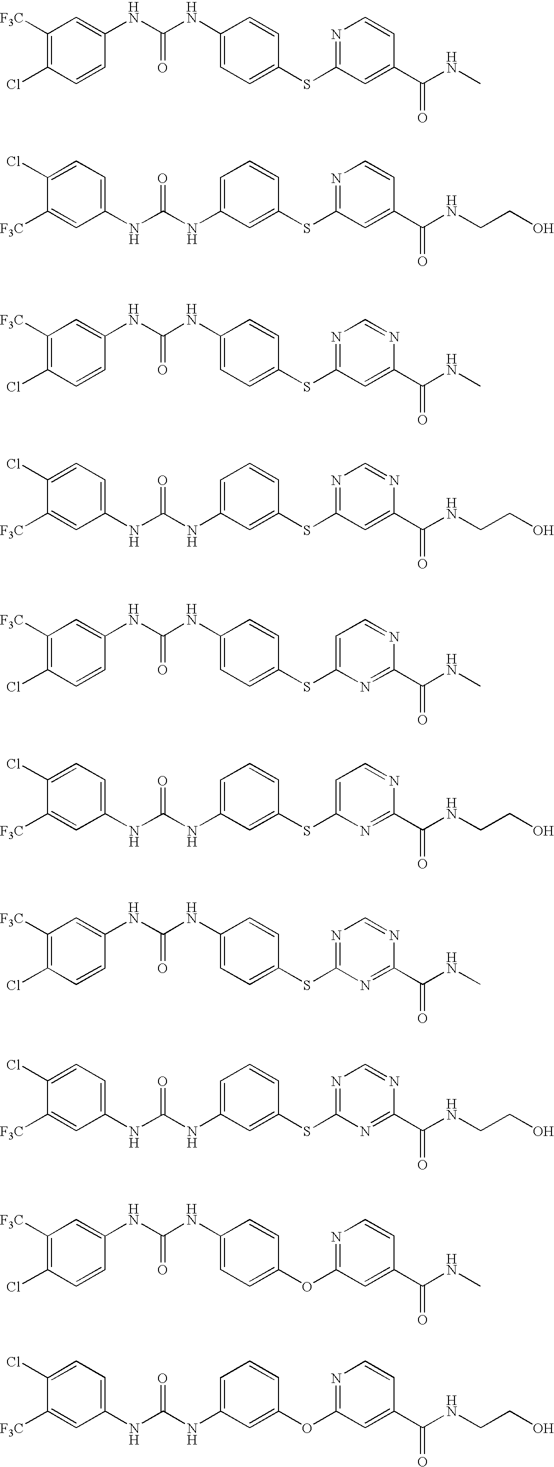

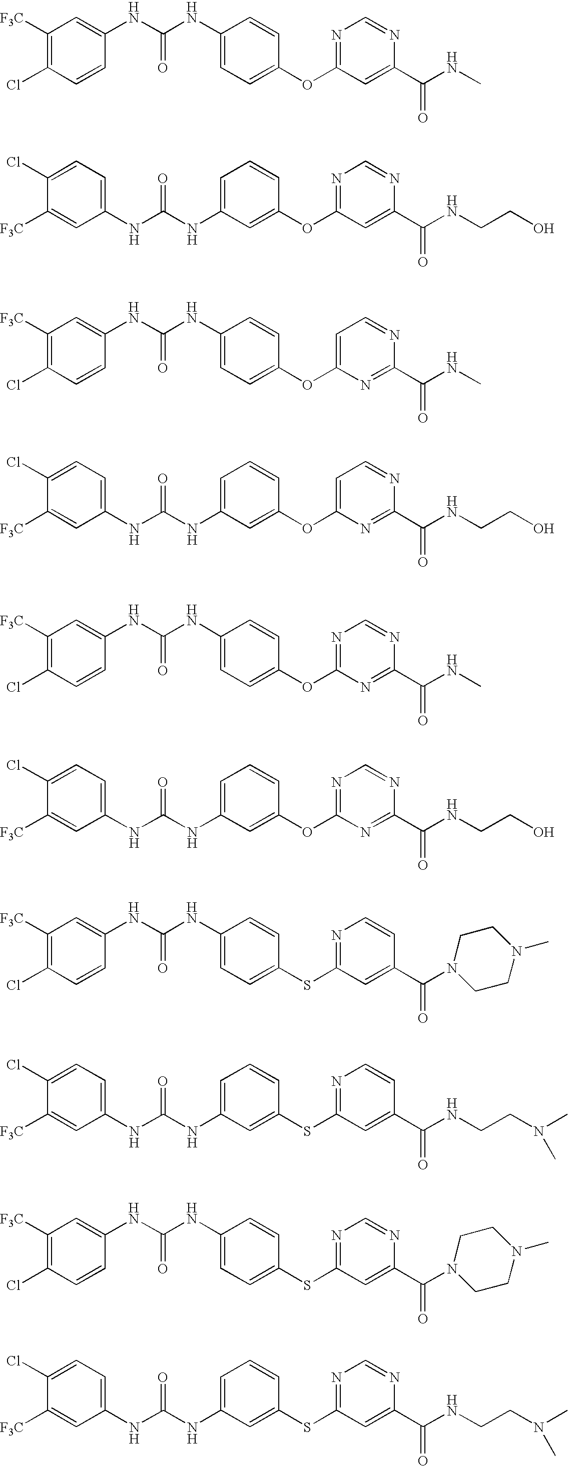

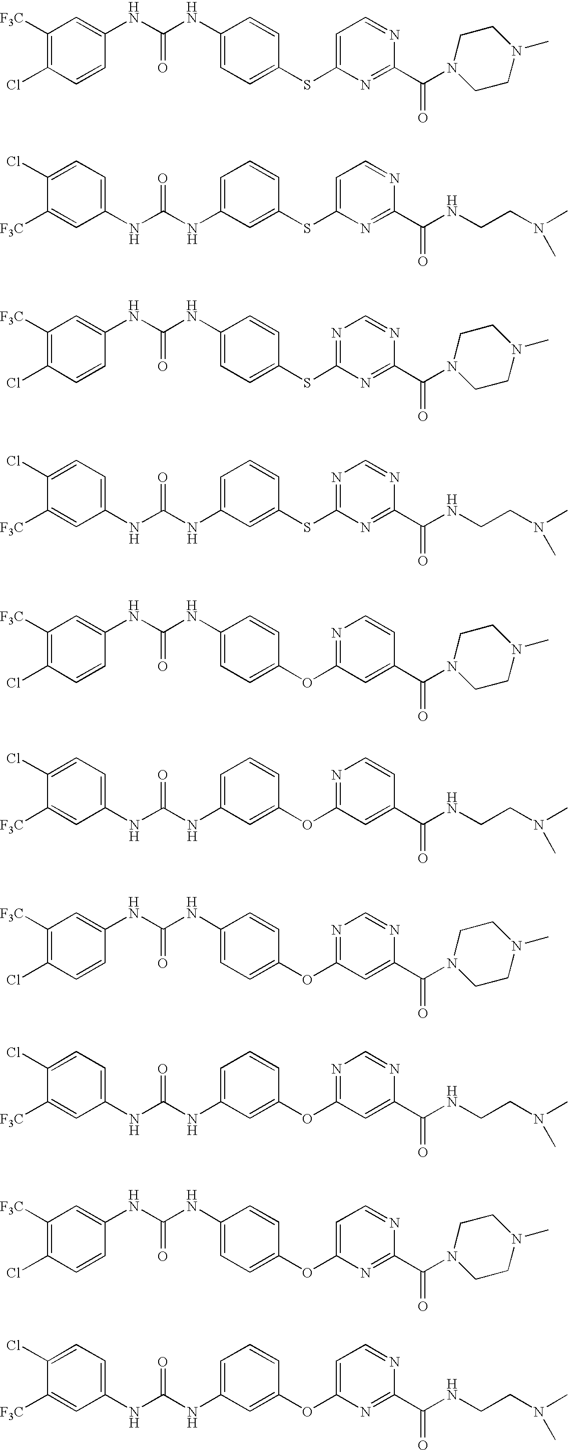

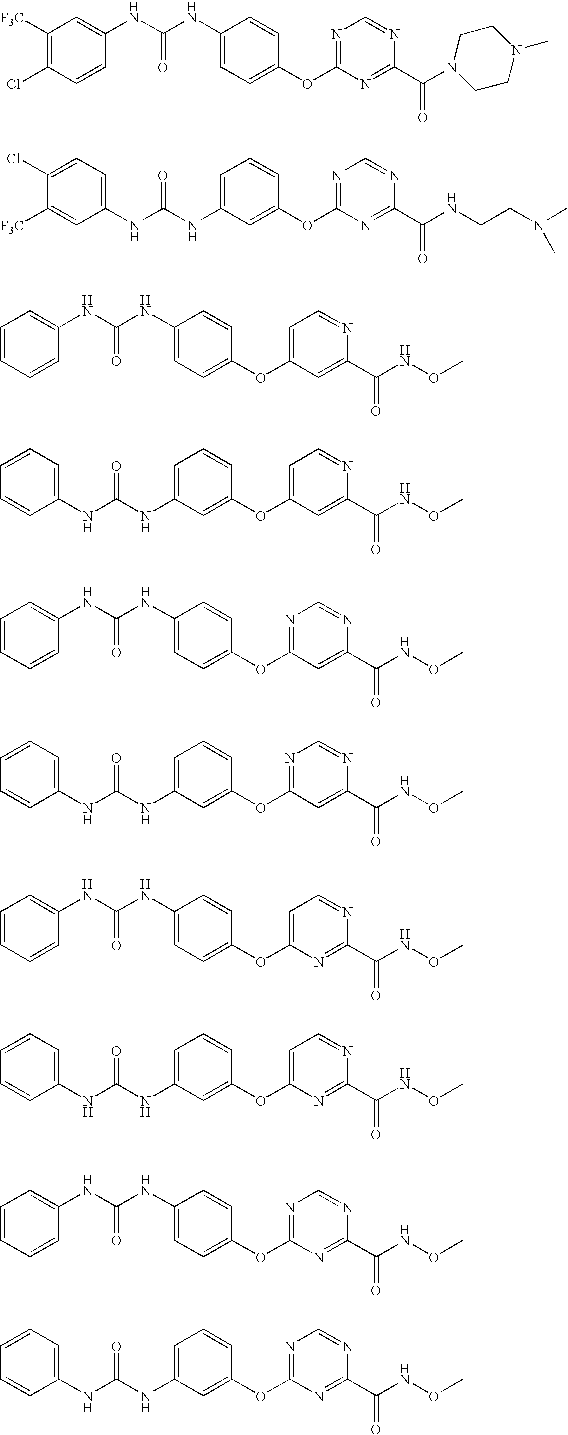

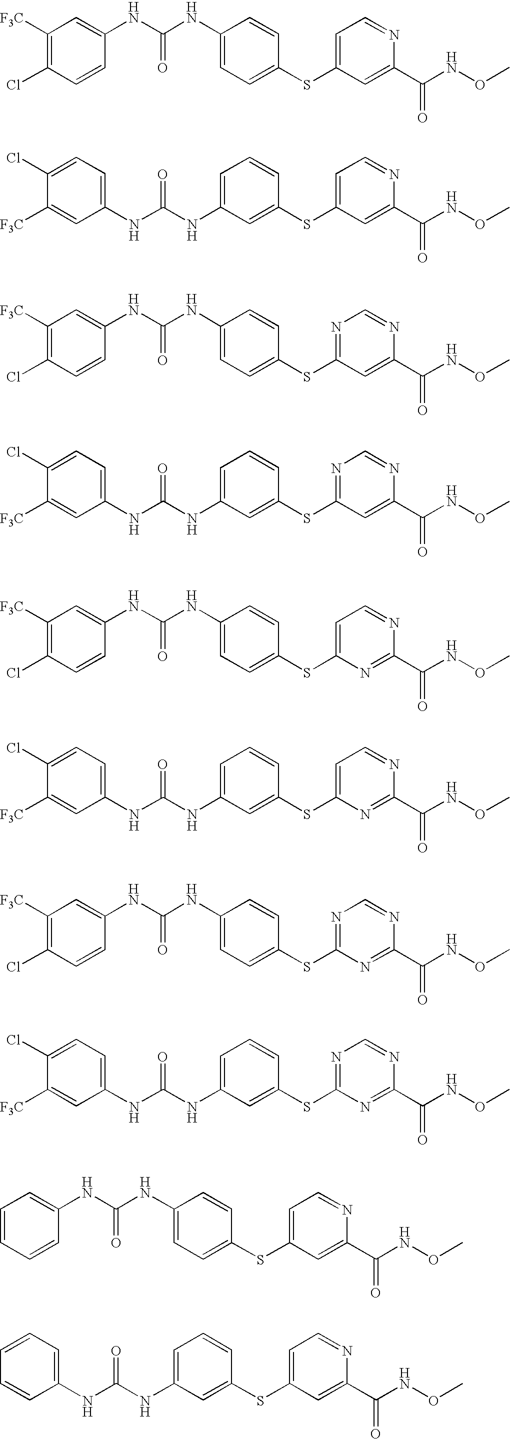

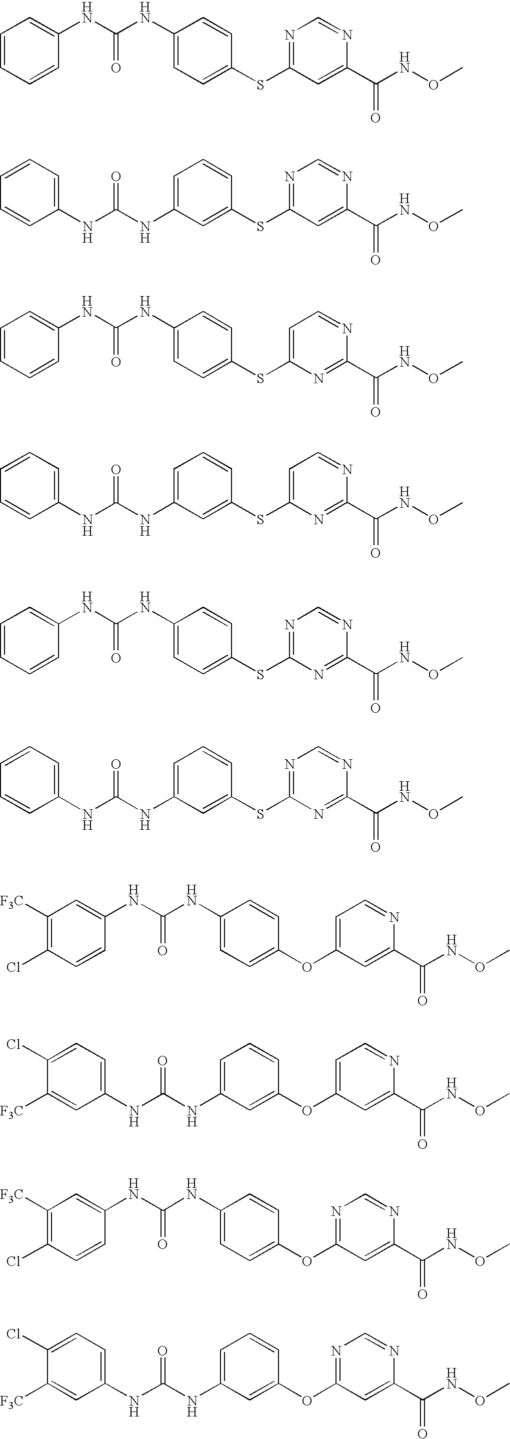

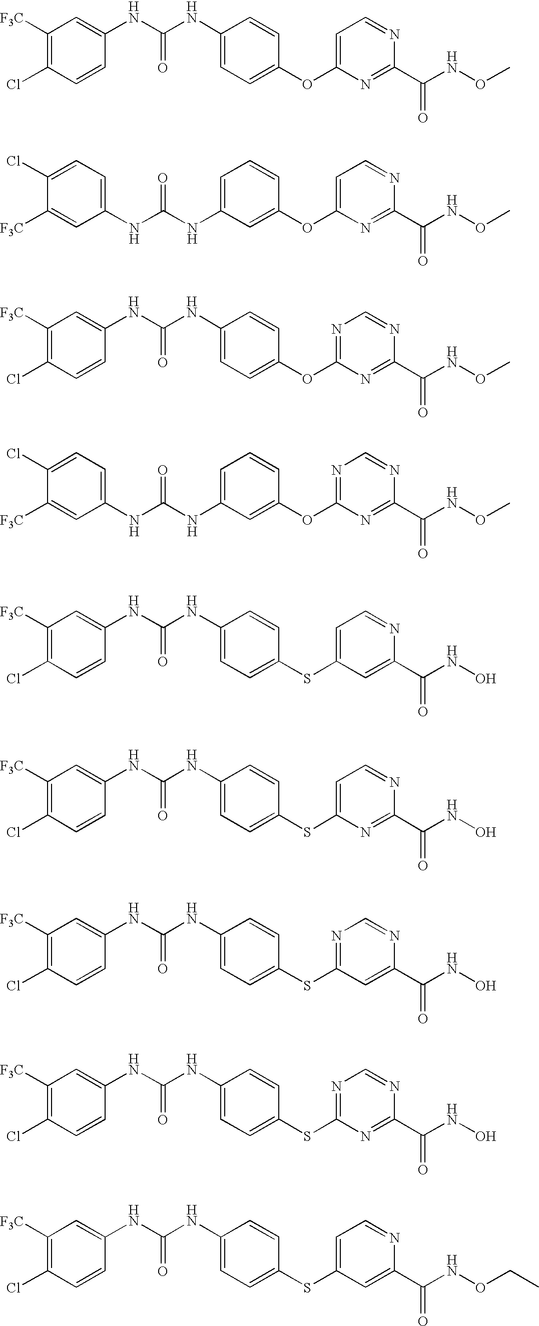

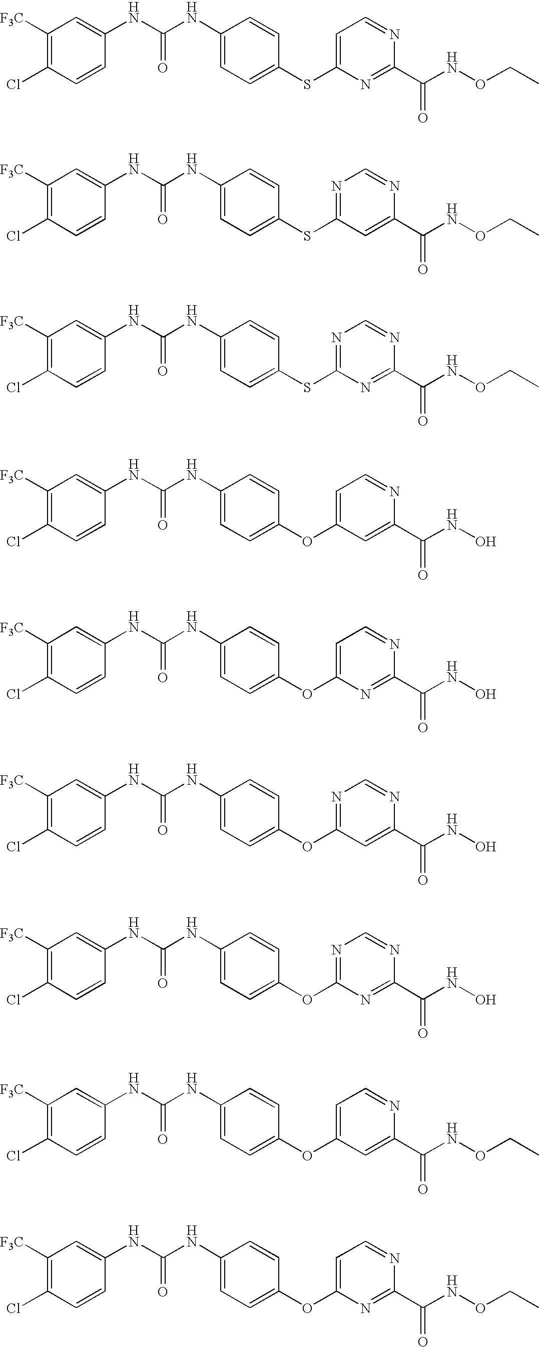

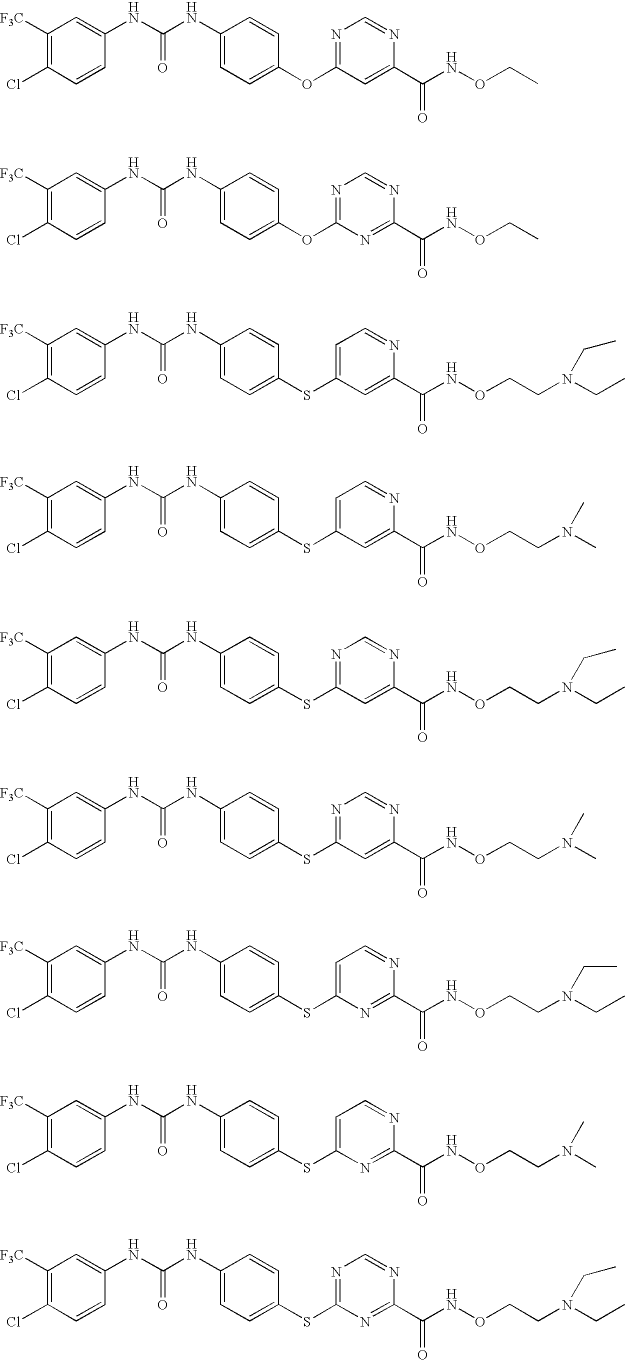

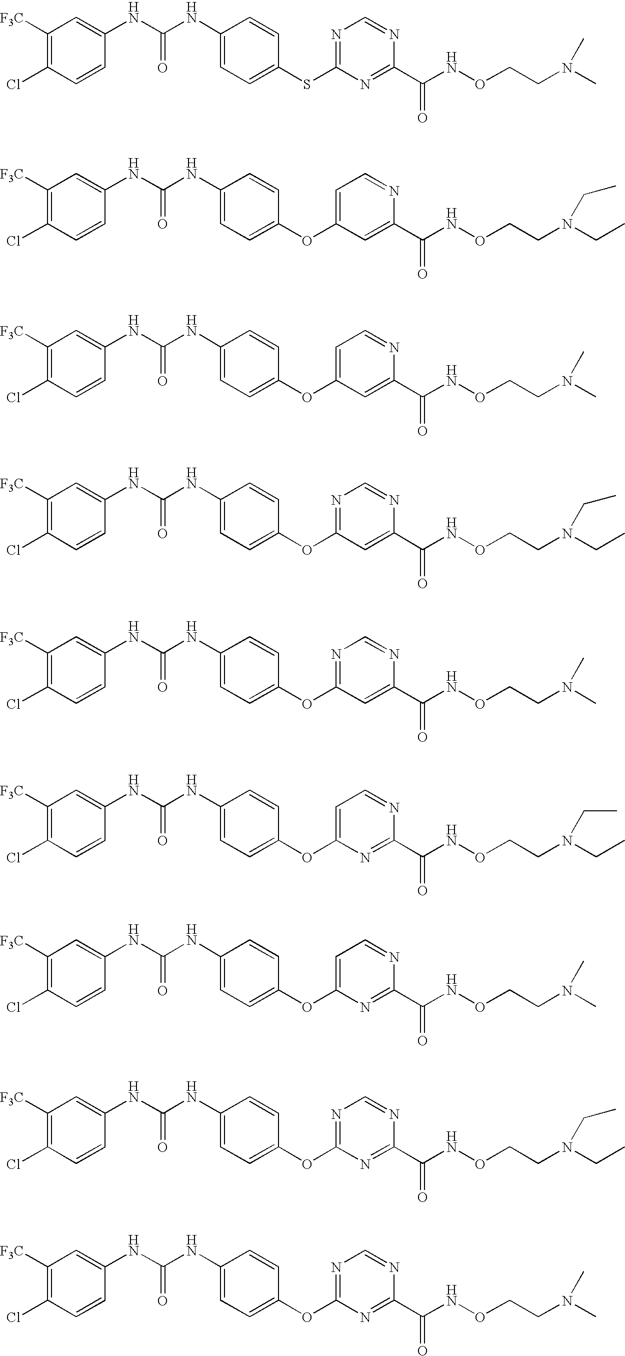

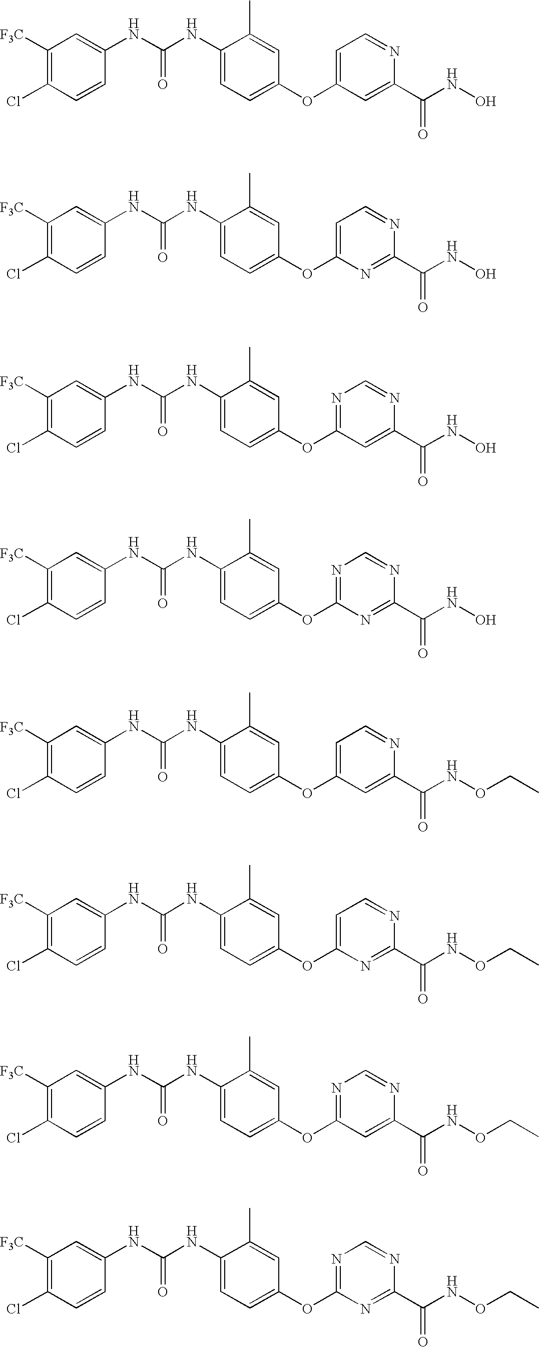

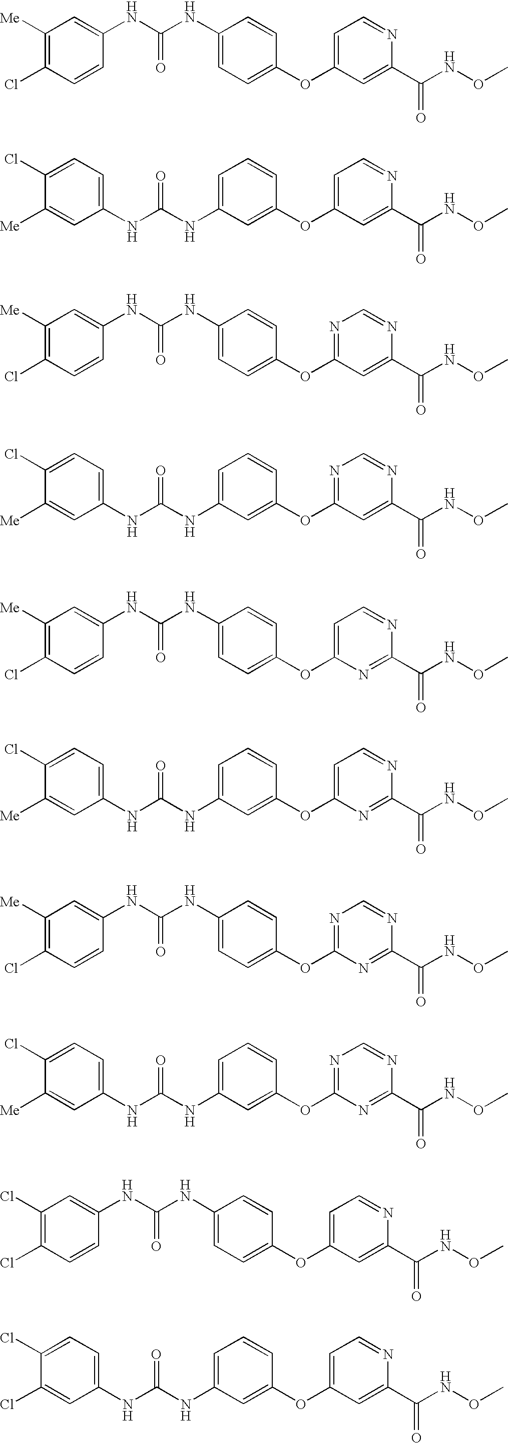

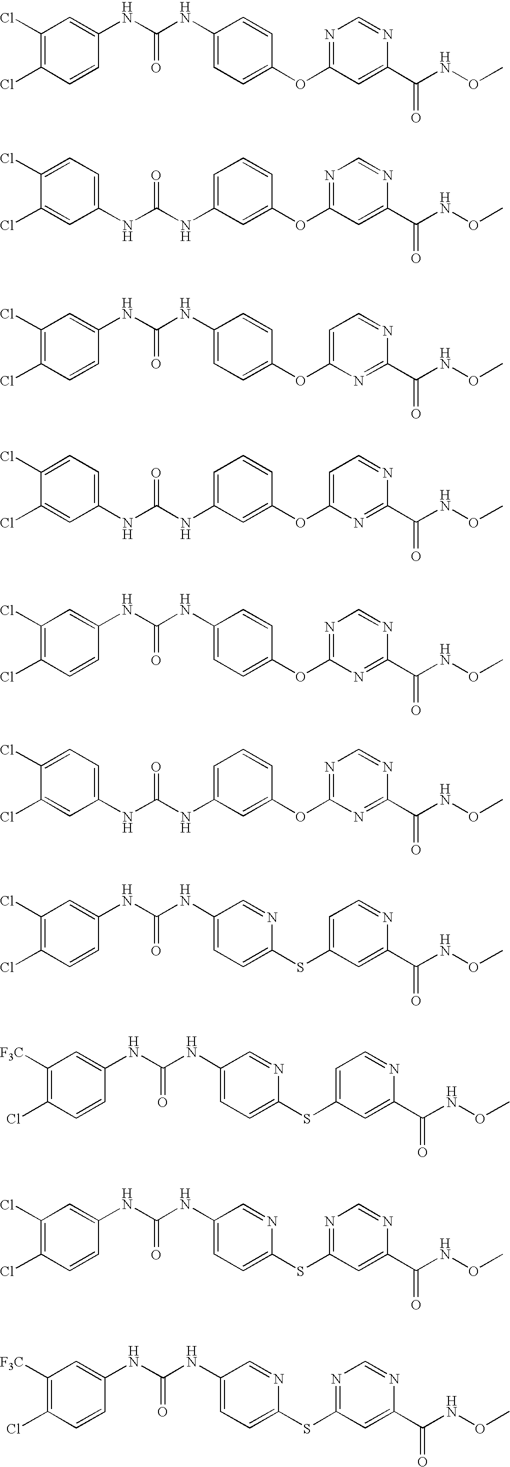

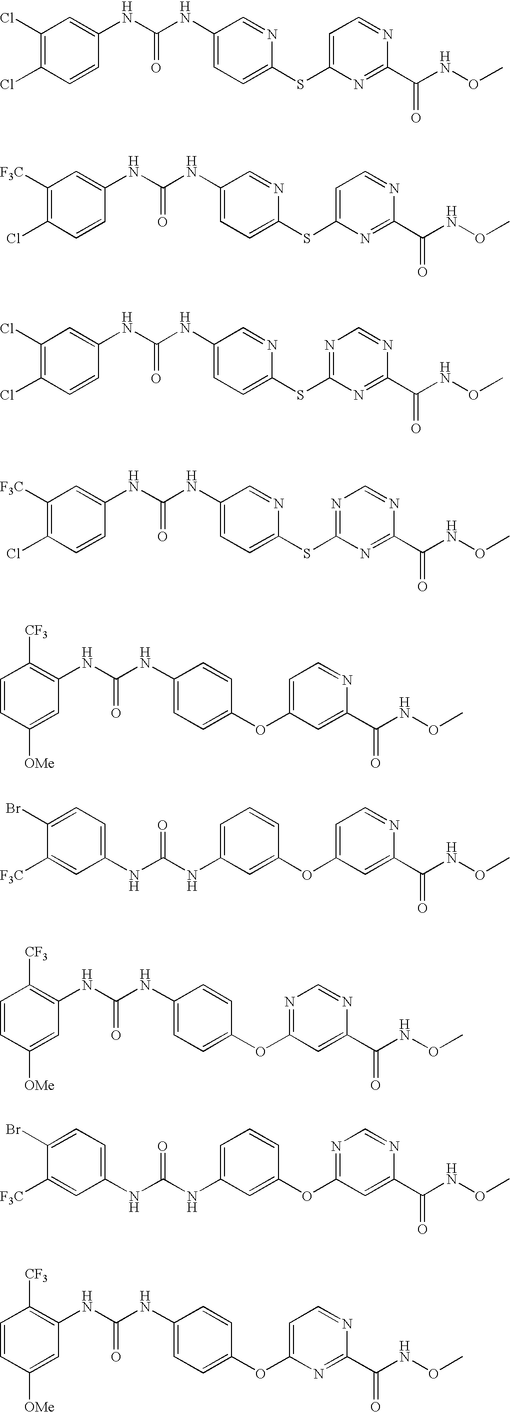

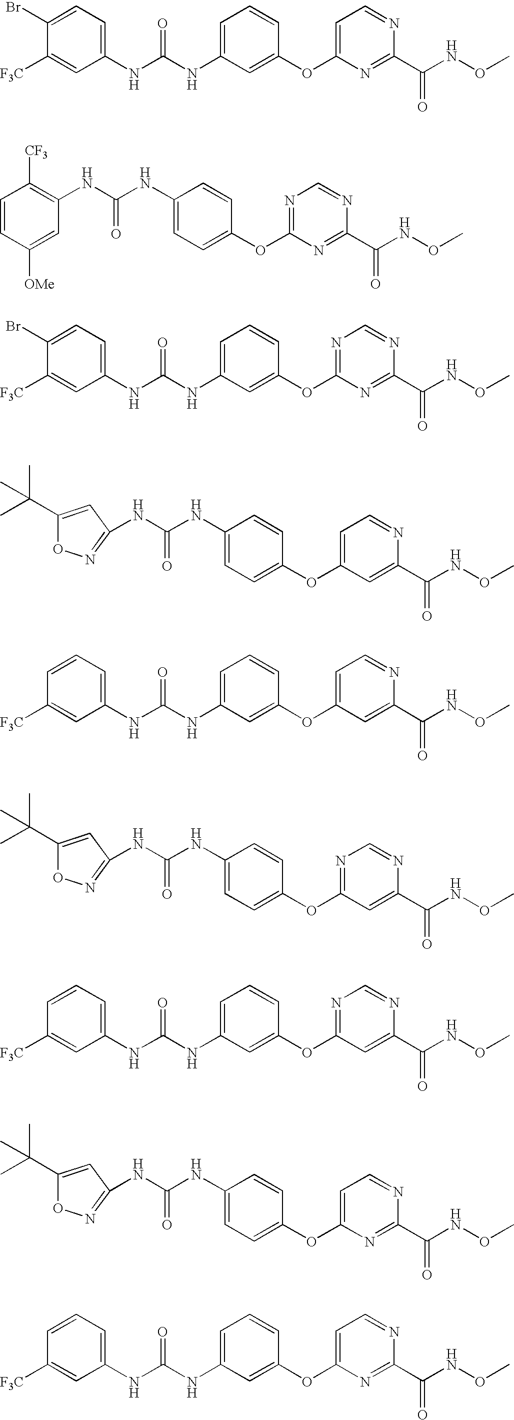

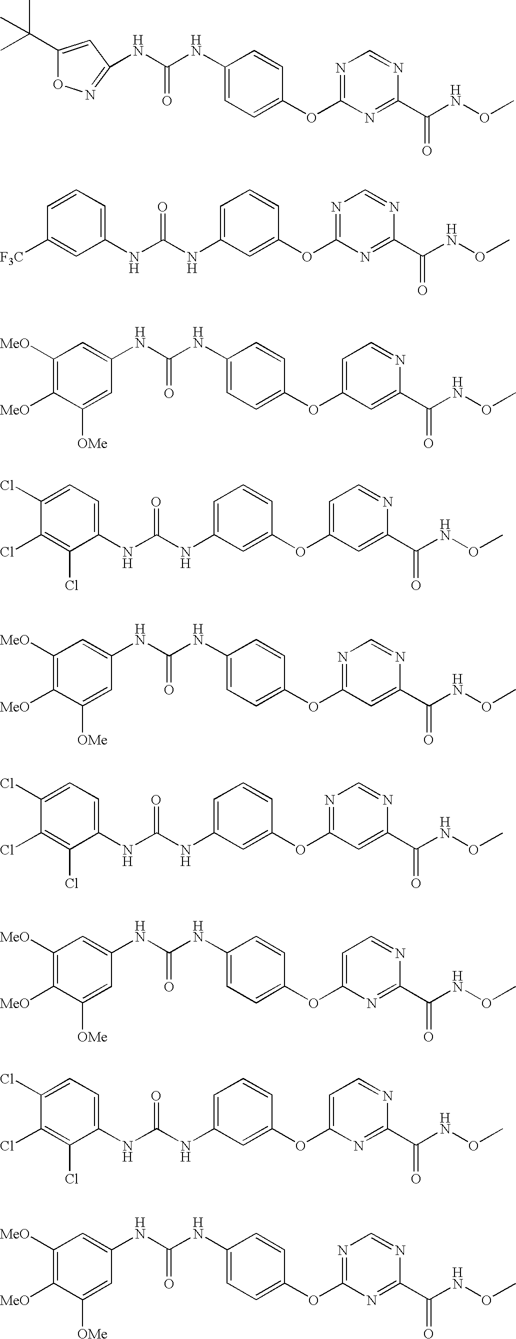

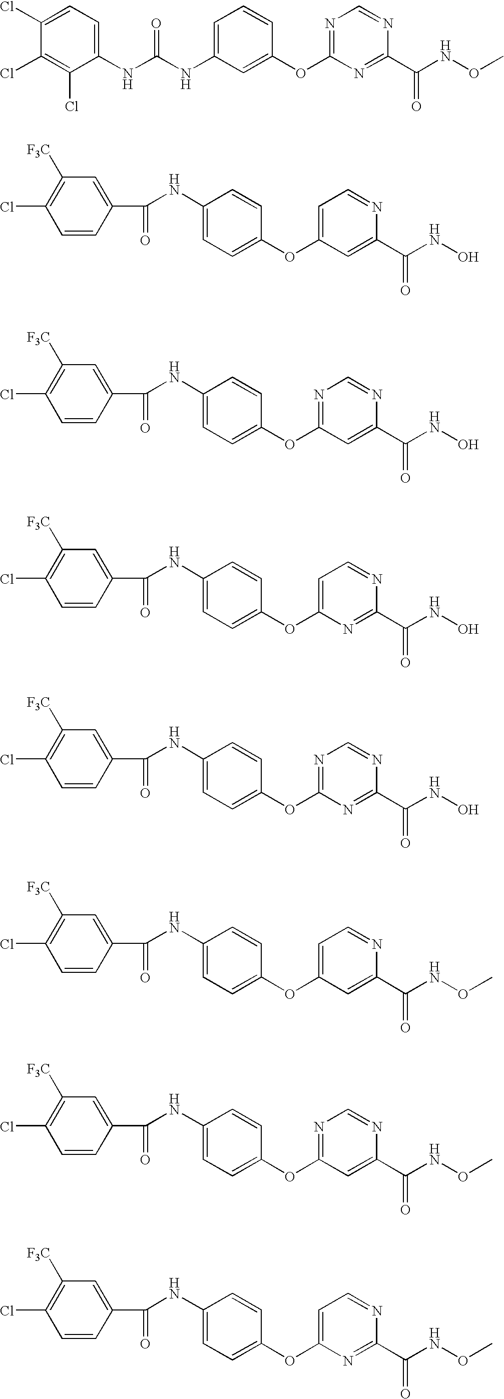

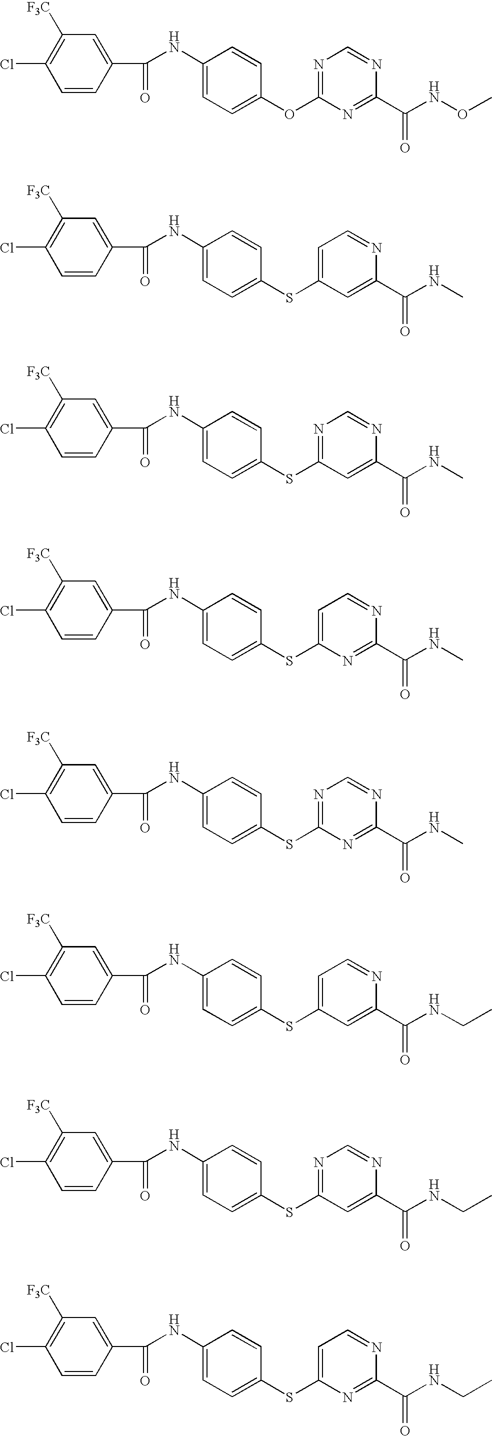

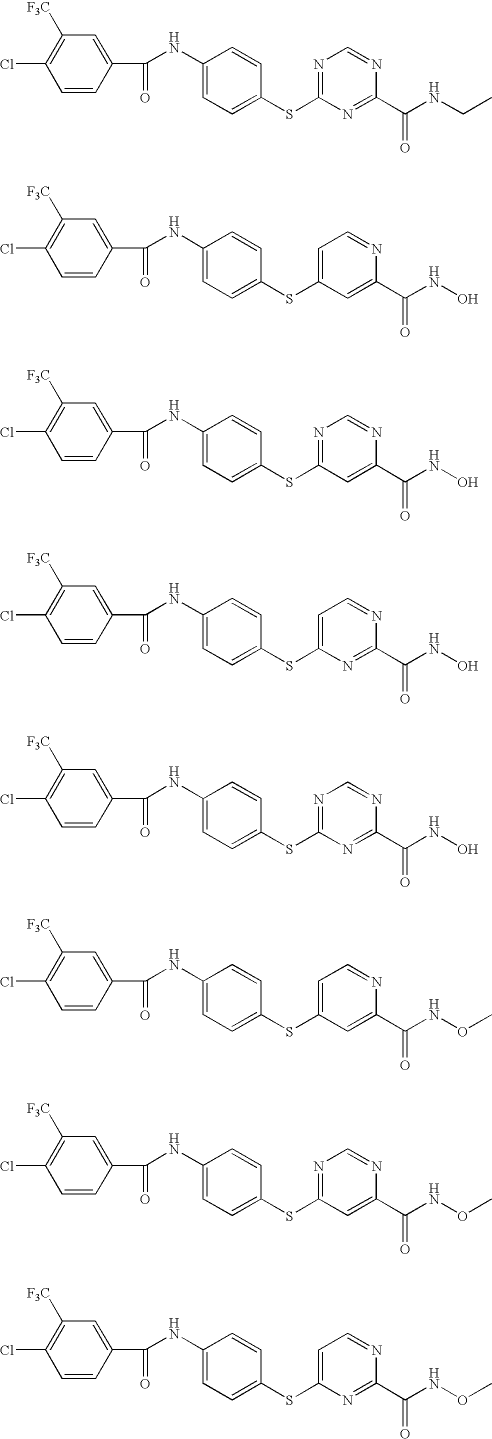

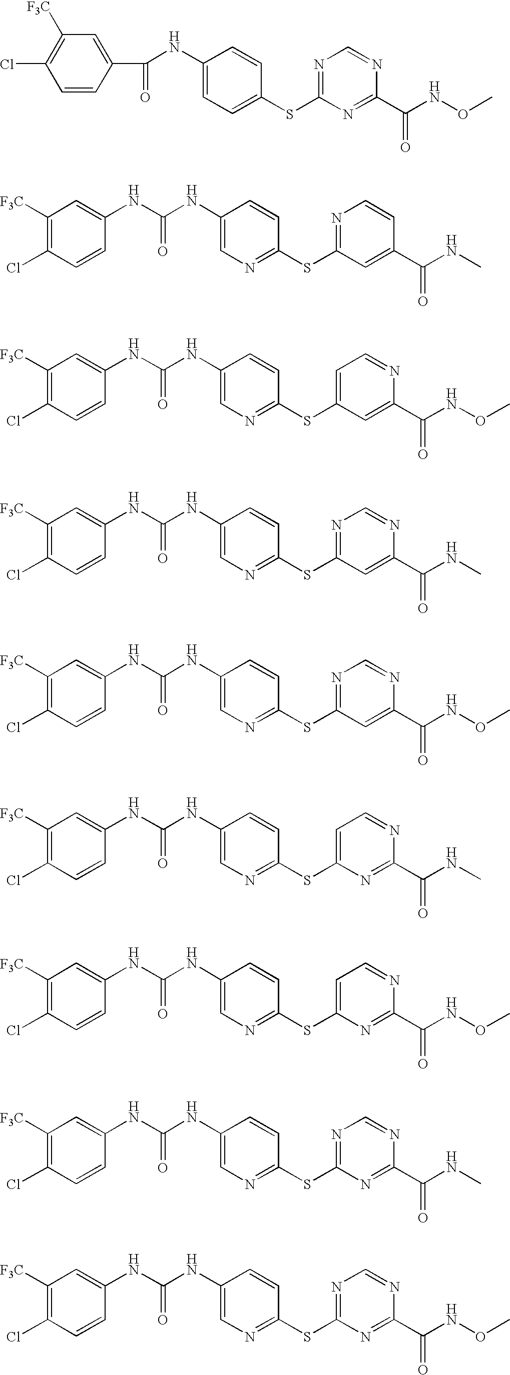

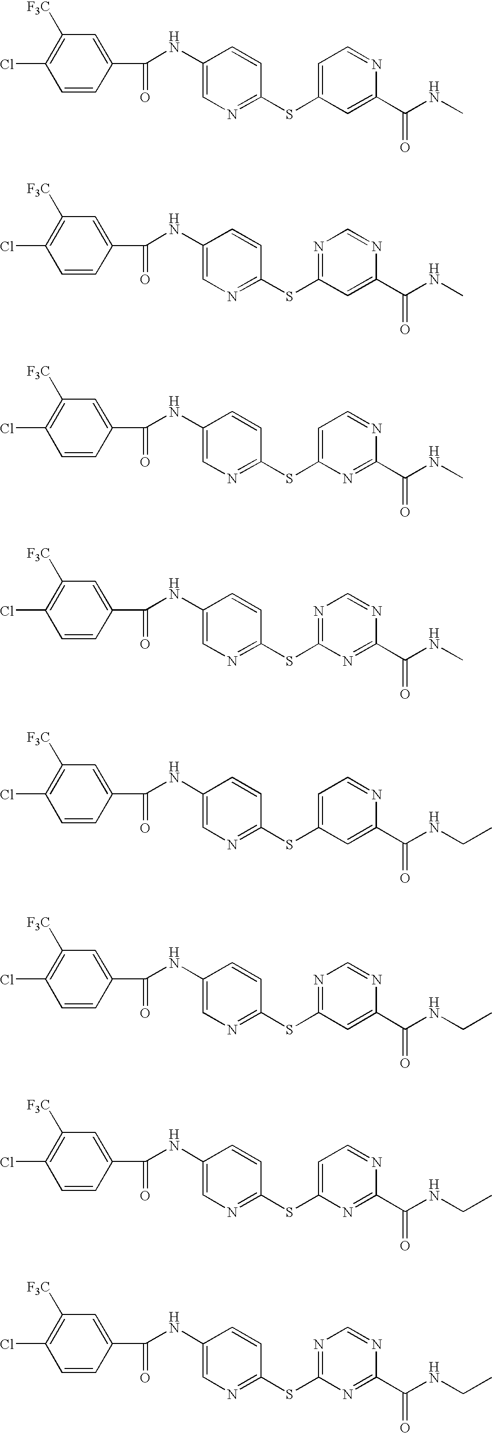

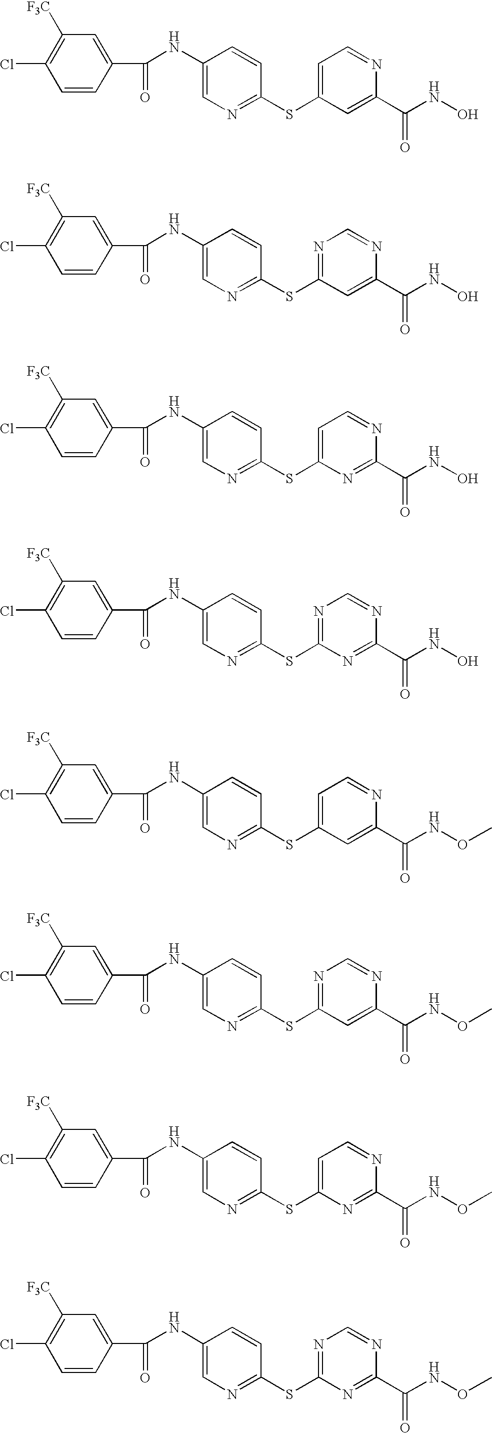

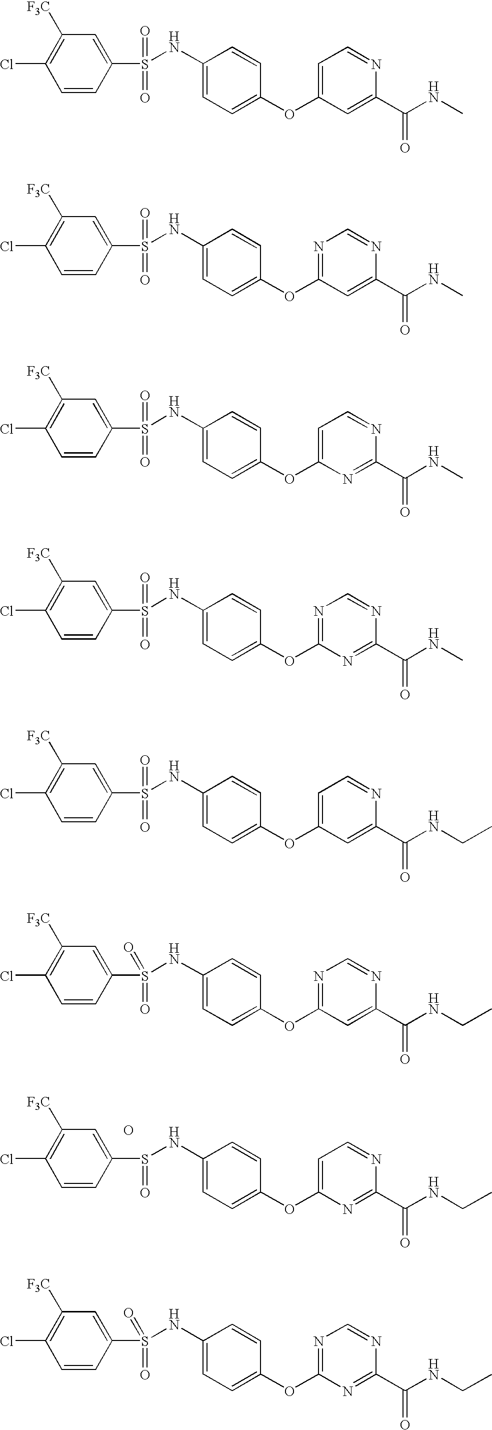

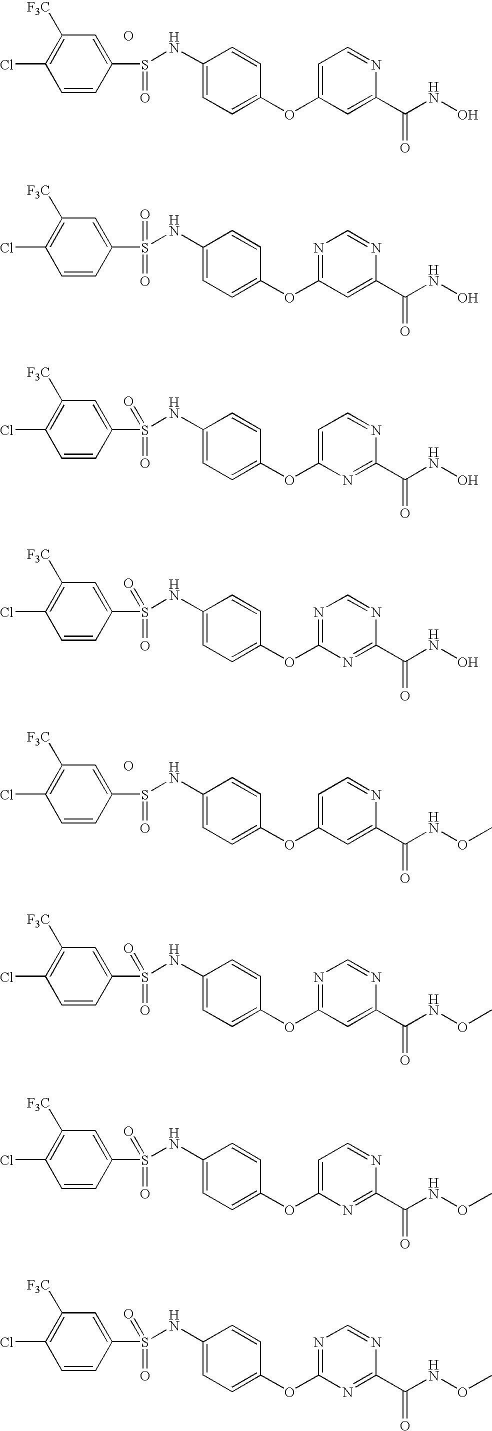

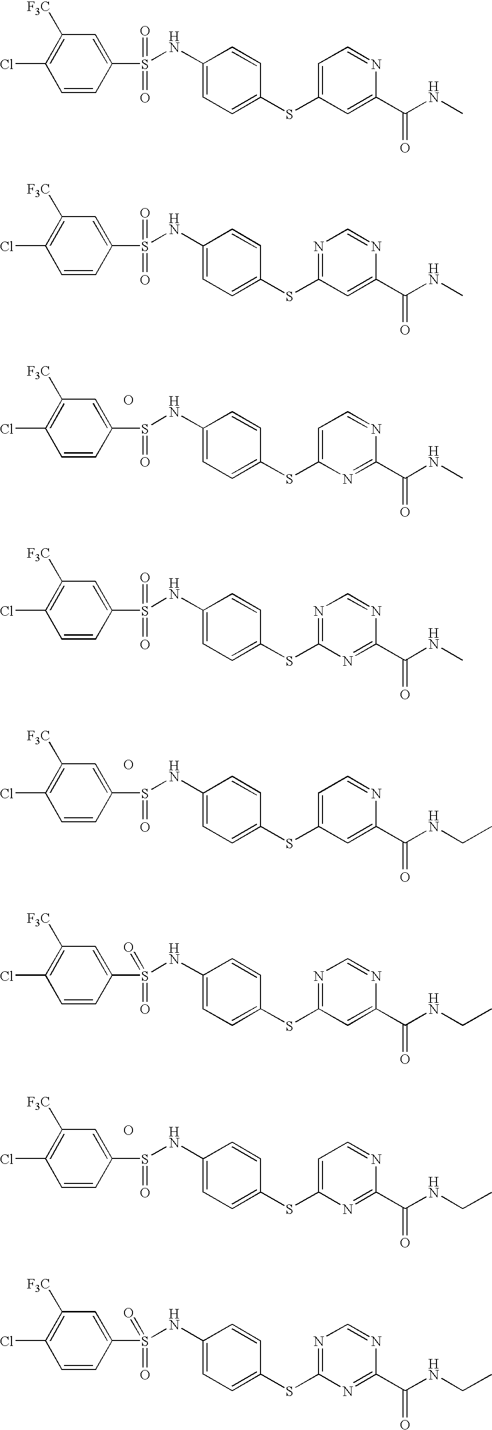

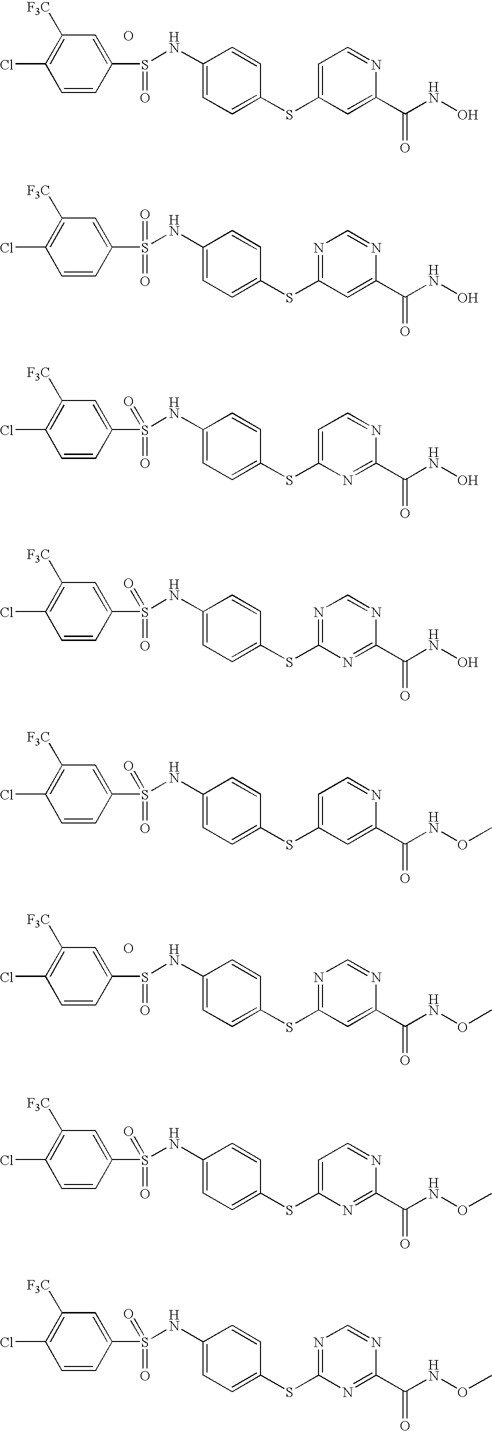

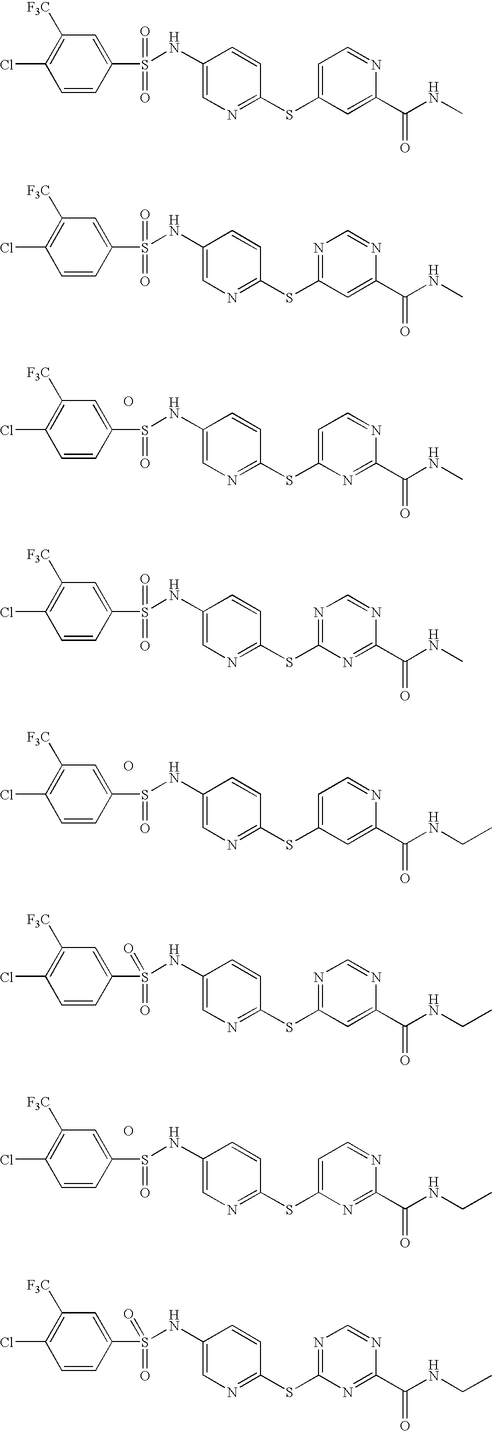

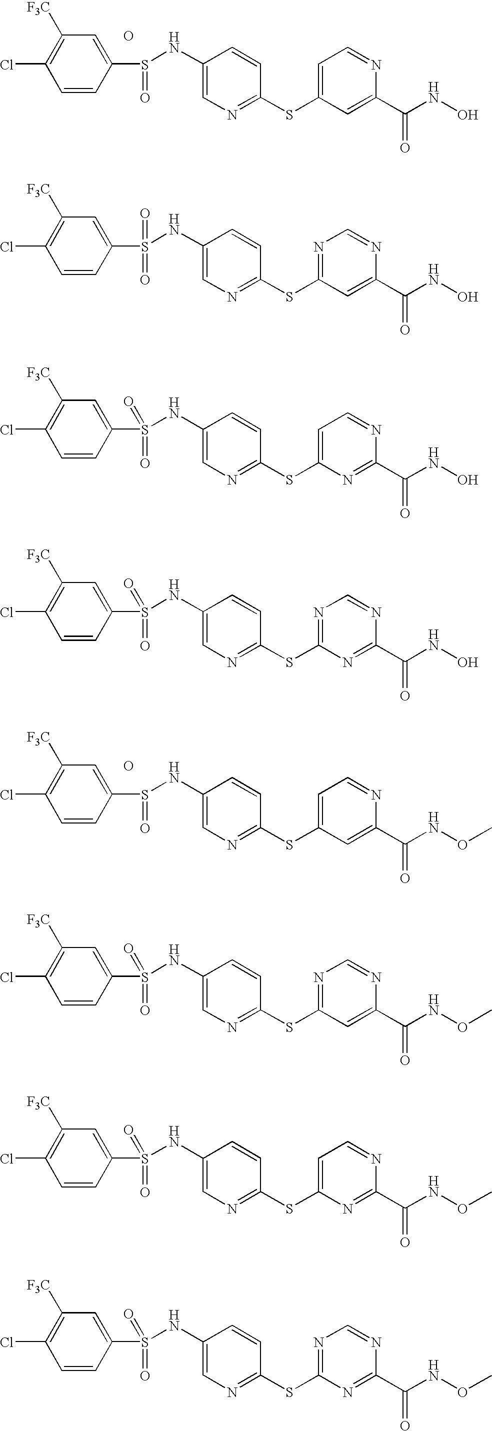

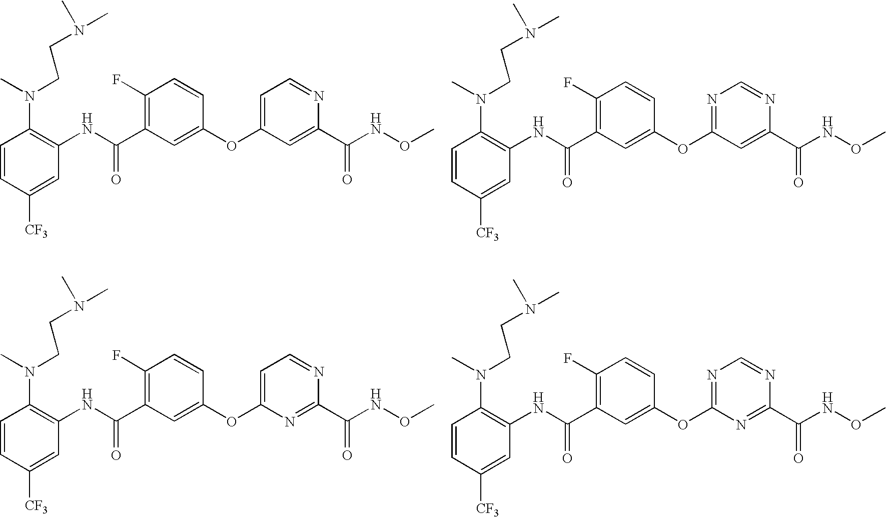

The compounds of Formula (I) preferably have a molecular weight of less than 800.

Particular compounds according to the invention include those mentioned in the examples and their pharmaceutically acceptable salts. It will be understood that the invention includes pharmaceutically acceptable derivatives of compounds of Formula (I) and that these are included within the scope of the invention.

As used herein “pharmaceutically acceptable derivative” includes any pharmaceutically acceptable salt, ester or salt of such ester of a compound of Formula (I) which, upon administration to the recipient, is capable of providing (directly or indirectly) a compound of Formula (I) or an active metabolite or residue thereof.

It will be appreciated that for use in medicine the salts of the compounds of Formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; or organic acids e.g. succinic, maleic, acetic, fumaric, citric, tartaric, benzoic, p-toluenesulfonic, methanesulfonic or naphthalenesulfonic acid. Other salts e.g. oxalates, may be used, for example, in the isolation of compounds of Formula (I) and are included within the scope of this invention.

The compounds of this invention may be in crystalline or non-crystalline form, and, if crystalline, may optionally be hydrated or solvated. This invention includes within its scope stoichiometric hydrates as well as compounds containing variable amounts of water.

The invention extends to all isomeric forms including stereoisomers and geometric isomers of the compounds of Formula (I) including enantiomers and mixtures thereof e.g. racemates. The different isomeric forms may be separated or resolved one from the other by conventional methods, or any given isomer may be obtained by conventional synthetic methods or by stereospecific or asymmetric syntheses.

The compounds of Formula (I) may be prepared singly or as compound libraries comprising at least 2, for example 5 to 1,000 compounds, and more preferably 10 to 100 compounds of Formula (I). Libraries of compounds of Formula (I) may be prepared by a combinatorial ‘split and mix’ approach or by multiple parallel synthesis using either solution phase or solid phase chemistry, by procedures known to those skilled in the art.

According to a further aspect of the invention there is provided methods for producing intermediates useful for the preparation of compounds of Formula (I) are provided, embodiments of said methods being depicted generally in Scheme 1.

Treatment of the chloro-pyrimidinecarboxilic acid with R1 functional group to give R1 substituted derivatives 2, which can be further processed with the difunctional K′—Ar₁—OH to give the ether linking key intermediates 3, which will further modified with the Ar₂ group to lead the desired products 5.

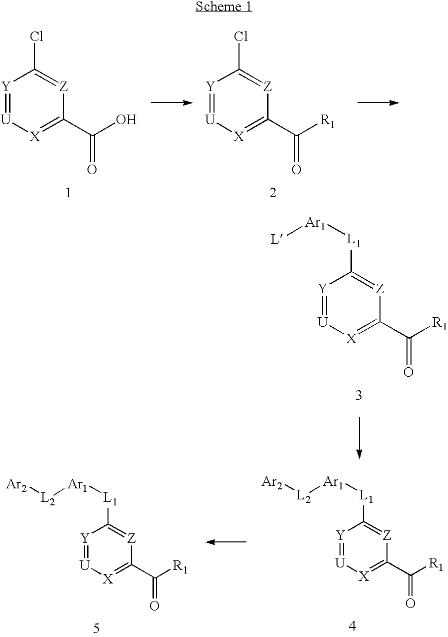

Alternatively, the product can be prepared in the procedure in Scheme 2. The temporary protect group of 3′ can be removed with the standard procedure to generated acid 4′, from which varies new R1 can be attached.

During the synthesis of the compounds of Formula (I) labile functional groups in the intermediate compounds, e.g. hydroxy, carboxy and amino groups, may be protected. A comprehensive discussion of the ways in which various labile functional groups may be protected and methods for cleaving the resulting protected derivatives is given in for example Protective Groups in Organic Chemistry, T. W. Greene and P. G. M. Wuts, (Wiley-Interscience, New York, 2nd edition, 1991). For example, t-butyl ester of compound 2 can be prepared with known procedure (Tetrahedron Lett. 37(24), 4237-4240,1996).

The representative Compound 1 of Scheme 1 can be prepared from the following responding commercial alcohol via the standard procedure reported in the prior art:

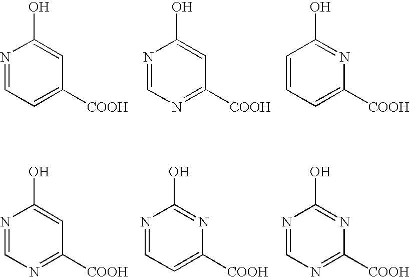

The one of the examples in the above can be synthesized according to the procedure in the literature (Scheme 2, Syn. Commun. 20(13), 2033-2040 (1990). For example, the treatment of formidamide and sodium (Z)-1,4-diethoxy-1,4-dioxobut-2-en-2-olate provides desired product 6,6-hydroxypyrimidine-4-carboxylic acid. The reaction of acid 6 and sulfurous dichloride yielded compound 7, which can be converted to carboxamide 8 readily.

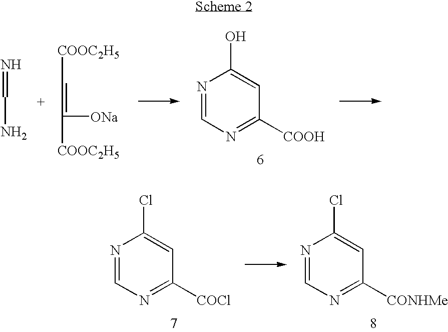

The present invention encompasses any prodrug form of the compounds described herein. Although certain other exemplary prodrug moieties generated from the inventive compounds amino group are detailed herein, it will be appreciated that the present invention is not intended to be limited to these prodrug moieties; rather, a variety of additional prodrug moieties can be readily identified by a person skilled in the relevant art.

As discussed above, the present invention provides compounds that are inhibitors of protein kinases (e.g., Raf kinase), and thus the present compounds are useful for the treatment of diseases, disorders, and conditions including, but not limited to melanoma, leukemia, or cancers such as colon, breast, gastric, ovarian, lung, brain, larynx, cervical, renal, lymphatic system, genitourinary tract (including bladder and prostate), stomach, bone, lymphoma, melanoma, glioma, papillary thyroid, neuroblastoma, and pancreatic cancer. Accordingly, in another aspect of the present invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

Compounds of the present invention may additionally be useful in the treatment of one or more diseases afflicting mammals which are characterized by cellular proliferation in the areas of blood vessel proliferative disorders, fibrotic disorders, mesangial cell proliferative disorders and metabolic diseases. Blood vessel proliferative disorders include arthritis and restenosis. Fibrotic disorders include hepatic cirrhosis and atherosclerosis. Mesangial cell proliferative disorders include glomerulonephritis, diabetic nephropathy, malignant nephrosclerosis, thrombotic microangiopathy syndromes, organ transplant rejection and glomerulopathies.

Metabolic disorders include psoriasis, diabetes mellitus, chronic wound healing, inflammation and neurodegenerative diseases.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

In order to use the compounds of Formula (I) in therapy, they will normally be formulated into a pharmaceutical composition in accordance with standard pharmaceutical practice.

According to a further aspect of the invention there is provided a pharmaceutical composition comprising a compound of Formula (I) or a pharmaceutically acceptable salt thereof and a pharmaceutically acceptable carrier.

The compounds of Formula (I) may be administered in conventional dosage forms prepared by combining it with standard pharmaceutical carriers according to conventional procedures. The compounds of Formula (I) may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable carrier is dictated by the amount of compound of Formula (I) with which it is to be combined, the route of administration and other well-known variables. The carrier(s) must be “acceptable” in the sense of being compatible with the other ingredients of the formulation and not deleterious to the recipient thereof.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Uses of Compounds and Pharmaceutically Acceptable Compositions

In one aspect, a method for the treatment or lessening the severity of an Raf-mediated disease or condition is provided comprising administering an effective amount of a compound, or a pharmaceutically acceptable composition comprising a compound to a subject in need thereof.

In certain embodiments of the present invention an “effective amount” of the compound or pharmaceutically acceptable composition is that amount effective for treating or lessening the severity of an Raf-mediated disease or condition. The compounds and compositions, according to the method of the present invention, may be administered using any amount and any route of administration effective for treating or lessening the severity of an Raf-mediated disease or condition. The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage.

The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The term “patient”, as used herein, means an animal, preferably a mammal, and most preferably a human.

The compounds of Formula (I) may conveniently be administered by any of the routes conventionally used for drug administration to humans and other animals orally, rectally, parenterally, intracisternally, intravaginally, intraperitoneally, topically (as by powders, ointments, or drops), bucally, as an oral or nasal spray, or the like, depending on the severity of the infection being treated.

Preferably, the pharmaceutically acceptable compositions of this invention may be orally administered in any orally acceptable dosage form including, but not limited to, capsules, tablets, troches, elixirs, suspensions, syrups, wafers, chewing gums, aqueous suspensions or solutions.

The oral compositions may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

For the purposes of parenteral therapeutic administration, the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

The compounds of Formula (I) may also be administered by inhalation, that is by intranasal and oral inhalation administration. Appropriate dosage forms for such administration, such as aerosol formulations, may be prepared by conventional techniques.

The compounds of Formula (I) may also be administered topically, that is by non-systemic administration. This includes the application of the inhibitors externally to the epidermis or the buccal cavity and the instillation of such a compound into the ear, eye and nose, such that the compound does not significantly enter the blood stream.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming micro encapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium.

Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of protein kinases. In one embodiment, the compounds and compositions of the invention are Raf kinase inhibitors, and thus, without wishing to be bound by any particular theory, the compounds and compositions are particularly useful for treating or lessening the severity of a disease, condition, or disorder where activation of Raf kinase is implicated in the disease, condition, or disorder. When activation of Raf kinase is implicated in a particular disease, condition, or disorder, the disease, condition, or disorder may also be referred to as “Raf-mediated disease” or disease symptom. Accordingly, in another aspect, the present invention provides a method for treating or lessening the severity of a disease, condition, or disorder where activation of Raf kinase is implicated in the disease state.

The activity of a compound utilized in this invention as an Raf kinase inhibitor, may be assayed in vitro, in vivo or in a cell line. In vitro assays include assays that determine inhibition of either the phosphorylation activity or ATPase activity of activated Raf. Alternate in vitro assays quantitate the ability of the inhibitor to bind to Raf. Inhibitor binding may be measured by radiolabelling the inhibitor prior to binding, isolating the inhibitor/Raf, complex and determining the amount of radiolabel bound. Alternatively, inhibitor binding may be determined by running a competition experiment where new inhibitors are incubated with RAF bound to known radioligands.

It will also be appreciated that the compounds and pharmaceutically acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

For example, other therapies, chemotherapeutic agents or other anti-proliferative agents may be combined with the compounds of this invention to treat proliferative diseases and cancer. Examples of therapies or anticancer agents that may be used in combination with the inventive anticancer agents of the present invention include surgery, radiotherapy (in but a few examples, gamma-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, adriamycin, dexamethasone, and cyclophosphamide. For a more comprehensive discussion of updated cancer therapies see the National Cancer Institute (CNI) website (www.nci.nih.gov) and the Food and Drug Administration (FDA) website for a list of the FDA approved oncology drugs (www.fda.gov).

Other examples of agents the inhibitors of this invention may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-i RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent.

Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the invention relates to inhibiting Raf activity in a biological sample or a patient, which method comprises administering to the patient, or contacting said biological sample with a compound of Formula (I) or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof

Inhibition of Raf kinase activity in a biological sample is useful for a variety of purposes that are known to one of skill in the art. Examples of such purposes include, but are not limited to, blood transfusion, organ-transplantation, biological specimen storage, and biological assays.

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

For all methods of use disclosed herein the daily oral dosage regimen will preferably be from about 0.1 to about 80 mg/kg of total body weight, preferably from about 0.2 to 30 mg/kg, more preferably from about 0.5 mg to 15 mg. The daily parenteral dosage regimen about 0.1 to about 80 mg/kg of total body weight, preferably from about 0.2 to about 30 mg/kg, and more preferably from about 0.5 mg to 15 mg/kg. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of the inhibitors will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of the inhibitors given per day for a defined number of days, can be ascertained by those skilled in the art using conventional course of treatment determination tests. In the case of pharmaceutically acceptable salts the above figures are calculated as the parent compound of Formula (I).

### EXAMPLES

The following Examples illustrate the preparation of pharmacologically active compounds of the invention and the following descriptions illustrate the preparation of intermediates used in the preparation of these compounds.

All experiments were performed under anhydrous conditions (i.e. dry solvents) in an atmosphere of argon, except where stated, using oven-dried apparatus and employing standard techniques in handling air-sensitive materials. Aqueous solutions of sodium bicarbonate (NaHCO3) and sodium chloride (brine) were saturated.

Analytical thin layer chromatography (TLC) was carried out on Merck Kiesel gel 60 F254 plates with visualization by ultraviolet and/or anisaldehyde, potassium permanganate or phosphomolybdic acid dips.

NMR spectra: 1H Nuclear magnetic resonance spectra were recorded at 500 MHz. Data are presented as follows: chemical shift, multiplicity (s=singlet, d=doublet, t=triplet, q=quartet, qn=quintet, dd=doublet of doublets, m=multiplet, bs=broad singlet), coupling constant (J/Hz) and integration. Coupling constants were taken and calculated directly from the spectra and are uncorrected.

Low resolution mass spectra: Electrospray (ES+) ionization was used. The protonated parent ion (M+H) or parent sodium ion (M+Na) or fragment of highest mass is quoted.

### Example 1

### This Example Showed the Synthesis of tert-butyl 4-chloropicolinate

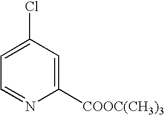

To 100 mL of THF was added 18 gm (240 mmol) of tert-butanol, while the solution was cooled into an ice-water bath, 30 mL (84 mmol) of 2.8 M nBuLi hexane solution was added slowly, and solution was warmed up to room temperature and 6.0 g (35.9 mmol) of methyl 4-chloropicolinate, which was prepared according to the procedure (Organic Process and Development, 2002, 6, p 777-781). The resulting mixture was stirred overnight and 100 mL of ethyl acetate was added. The mixture was washed once with brine solution and organic layer was dried over Na₂SO₄ and concentrated. The product was purified on silica gel, eluting with 0-30% of Ethyl acetate/hexane, affording 3.8 gm (49.6%) desired product. ¹HNMR (300 MHz, DMSO-d6), δ 1.50 (s, 9H, t-butyl), 7.81 (dd, J=5.4 Hz, 2.4 Hz, 1H, Ar), 8.06 (d, J=2.4 Hz, 1H, Ar), 8.68 (d, J=5.4 Hz).

### Example 2

### This Example Showed the Synthesis of tert-butyl 4-(4-aminophenoxy)picolinate

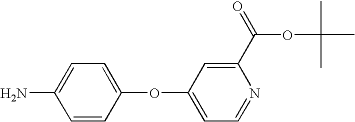

To 2.3 gm (21.1 mmol) of 4-hydroxyaniline was added 40 mL of dry DMF, and then 2.36 gm (21.1 mmol) of potassium tert-butoxide was added and the reaction mixture was stirred for 30 min, and then 4.5 gm (21.1 mmol) of tert-butyl 4-chloropicolinate, prepared as in Example 1, was added, followed by adding 0.2 gm (1.5 mmol) of potassium carbonate. The reaction mixture was heated to 80° C. and stirred for 1 h. After cooling to room temperature, 300 mL of ethyl acetate and 200 mL of the brine were added, after shaking well, organic layer was separated and washed once with the brine, and dried over sodium sulfate and concentrated. The product was further purified on the silica gel, eluting with 0-50% ethyl acetate/hexane to give 2.0 gm (50.9%) of desired product. ¹HNMR (300 MHz, DMSO-d6), δ 1.50 (s, 9H, t-butyl), 5.17 (s, 2H, NH2), 6.46-6.86 (AA′BB′, quartet, J=8.4 Hz, Ar), 7.02 (dd, J=5.7 Hz, 2.4 Hz, 1H, Ar), 7.33 (d, J=2.4 Hz, 1H, Ar), 8.47 (d, J=5.7 Hz, 1H, Ar). Mass spectrum, m/e=287.2 (M+H)⁺.

### Example 3

### This Example Showed the Synthesis of tert-butyl 4-(4-(3-(4-chloro-3-(trifluoromethyl)phenyl)ureido)phenoxy)picolinate

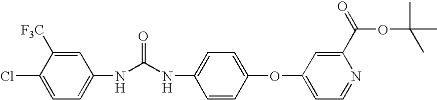

To 1.3 gm (4.54 mmol) of tert-butyl 4-(4-aminophenoxy)picolinate, prepared in Example 2, was added 15 mL of dry CH₂Cl₂, while the solution was cooled into an ice-water bath, a solution of 1.2 gm (5.41 mmol) of 4-chloro-3-(trifloromethyl)phenyl isocynate in 5 mL of dry CH₂Cl₂. The reaction mixture was stirred overnight under N2. Silica gel (120 gm) was loaded with hexane, and the crude product was loaded, the column was eluted with 200 mL of hexane, 500 mL of 30% ethyl acetate/hexane and 2×500 mL of 50% ethyl acetate/hexane. The desired fractions were pooled and concentrated to give 1.70 gm (73.7%) of the desired compound. ¹HNMR (300 MHz, DMSO-d6), δ 1.50 (s, 9H, t-butyl), 7.10 (dd, J=6.6 Hz, 2.7 Hz, 1H, Ar), 7.15 (d, J=8.7 Hz, 2H, Ar), 7.39 (d, J=2.4 Hz, 1H, Ar), 7.55-7.66 (m, 4H, Ar), 8.10 (d, J=2.4 Hz, 1H, Ar), 8.53 (d, J=5.7 Hz, 1H, Ar), 8.99 (s, 1H, CONH) and 9.20 (s, 1H, CONH), Mass spectrum, m/e=508.3 (M+H)⁺.

### Example 4

### This Example Showed the Synthesis of 4-(4-(3-(4-chloro-3-(trifluoromethyl)phenyl)ureido)phenoxy)picolinic acid

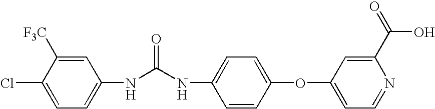

To 1.2 gm (2.37 mmol) of tert-butyl 4-(4-(3-(4-chloro-3-(trifluoromethyl)phenyl)ureido)phenoxy)picolinate, prepared in Example 3, was added a mixture of 35 mL of CH₂Cl₂/35 mL of TFA containing 0.1 mL of triisopropylsilane. After shaking for 10 min, 50 mL of toluene was added and the reaction mixture was concentrated and co-evaporated once with toluene. The crude product was dissolved in the small amount of methanol, followed by adding 50 mL of ether, the top solution was decanted and the gum product was dried over a high vacuum and give about 1.2 gm of the crude desired product, without further purification, which was used for the next step reaction.

### Example 5

### This Example Showed the synthesis of 4-(4-(3-(4-chloro-3-(trifluoromethyl)phenyl)ureido)phenoxy)-N-methoxypicolinamide

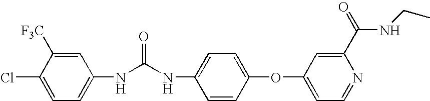

To 0.5 gm (1.1) mL of the crude 4-(4-(3-(4-chloro-3-(trifluoromethyl)phenyl)ureido)phenoxy)picolinic acid, prepared in Example 4, was added 30 mL of dry DMF, and 0.66 gm (3.44 mmol) of EDAC and 0.46 gm (3.44 mmol) of HOBT and 1.0 mL of DIPEA under nitrogen. After stirring for 1 h, 0.8 gm (9.6 mmol) of methoxyamine hydrochloride was added and the reaction mixture was stirred overnight. Into this mixture was added 200 mL of ethyl acetate and 60 mL of the brine solution, shaking well and organic layer was separated and washed once with 30 mL of 0.1 M aqueous citric acid/30 mL of the brine, and once with 100 mL of the brine solution, dried over sodium sulfate and concentrated. The product was further purified on the silica gel column, eluting with 0-50% ethyl acetate/hexane. The desired fractions were pooled and concentrated to give 0.4 gm (75.6%) of the pure desired compound. ¹HNMR (300 MHz, DMSO-d6), δ 3.31 (s, 3H, OCH3), 7.15-7.18 (m, 3H, Ar), 7.31 (d J=2.4 Hz, 1H, Ar), 7.56-7.66 (m, 4H, Ar), 8.11 (d, J=2.4 Hz, 1H, Ar), 8.47 (d, J=6.0 Hz, 1H, Ar.), 9.00 (s, 1H, CONH), 9.21 (s, 1H, Ar), 12.04 (s, 1H, NHOCH3). Mass spectrum, m/e=481.2 (M+1)⁺, 503.2 (M+Na)⁺.

The compounds of examples 6-28 in Table 1 were prepared by the general method described in Example 1-5.

TABLE 1

| | | | MS |
|---|---|---|---|
| | | | (M + |
| Ex. | | | H), |
| No. | Structure | Chemical name | m/e |
| 6 | 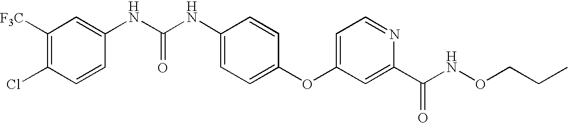 | 4-(4-(3-(4-chloro-3-(trifluoromethyl)phenyl)urei-do)phenoxy)-N-propoxypicolinamide | 509 |
| 7 | 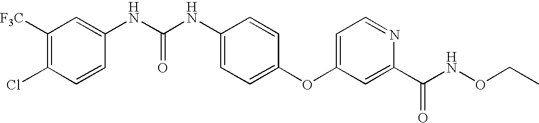 | 4-(4-(3-(4-chloro-3-(trifluoromethyl)phenyl)urei-do)phenoxy)-N-ethoxypicolinamide | 495 |
| 8 | 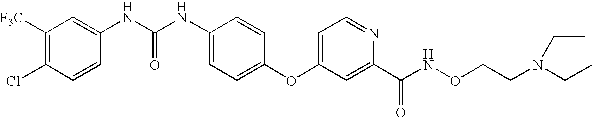 | 4-(4-(3-(4-chloro-3-(trifluoromethyl)phenyl)urei-do)phenoxy)-N-(2-(diethylamino)ethoxy)picolinamide | 566 |
| 9 | 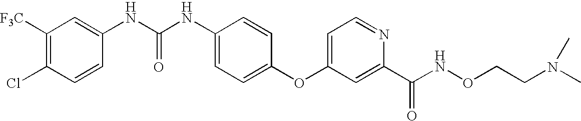 | 4-(4-(3-(4-chloro-3-(trifluoromethyl)phenyl)urei-do)phenoxy)-N-(2-(dimethylamino)ethoxy)picolinamide | 538 |
| 10 | 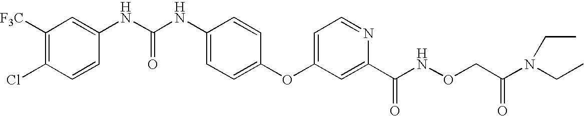 | 4-(4-(3-(4-chloro-3-(trifluoromethyl)phenyl)urei-do)phenoxy)-N-(2-(diethylamino)-2-oxoethoxy)picolinamide | 580 |
| 11 | 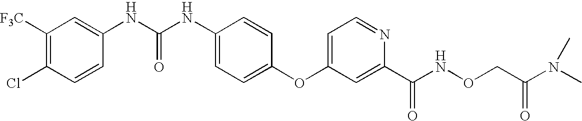 | 4-(4-(3-(4-chloro-3-(trifluoromethyl)phenyl)urei-do)phenoxy)-N-(2-(dimethylamino)-2-oxoethoxy)picolinamide | 552 |
| 12 | 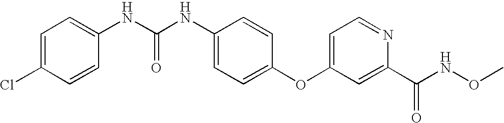 | 4-(4-(3-(4-chlorophenyl)ureido)phenoxy)-N-methoxypicolinamide | 413 |
| 13 | 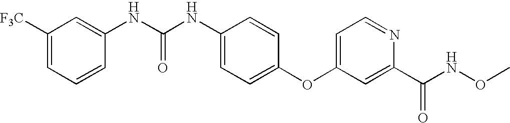 | N-methoxy-4-(4-(3-(3-(trifluoromethyl)phenyl)urei-do)phenoxy)picolinamide | 447 |
| 14 | 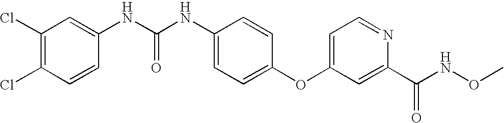 | 4-(4-(3-(3,4-dichlorophenyl)ureido)phenoxy)-N-methoxypicolinamide | 447 |
| 15 | 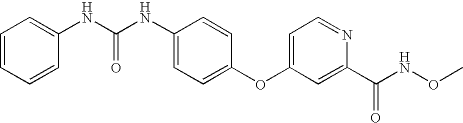 | N-methoxy-4-(4-(3-phenylureido)phenoxy)picolinamide | 379 |
| 16 | 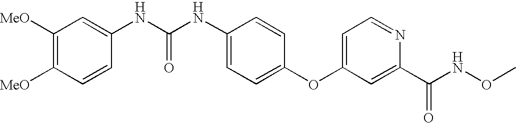 | 4-(4-(3-(3,4-dimethoxyphenyl)ureido)phenoxy)-N-methoxypicolinamide | 439 |
| 17 | 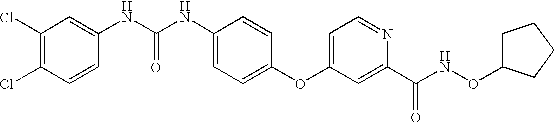 | N-(cyclopentyloxy)-4-(4-(3-(3,4-dichloro-phenyl)ureido)phenoxy)picolinamide | 502 |
| 18 | 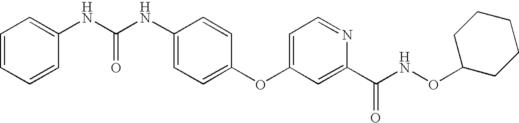 | N-(cyclohexyloxy)-4-(4-(3-phenylureido)phenoxy)picolinamide | 447 |
| 19 | 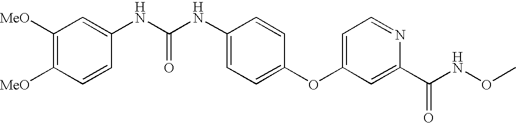 | N-cyclobutoxy-4-(4-(3-(3,4-dimethoxyphenyl)urei-do)phenoxy)picolinamide | 479 |
| 20 | 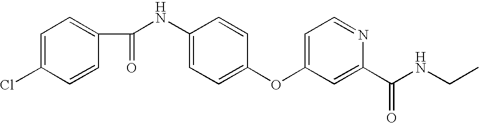 | 4-(4-(4-chloro-benzamido)phenoxy)-N-ethylpicolinamide | 397 |
| 21 | 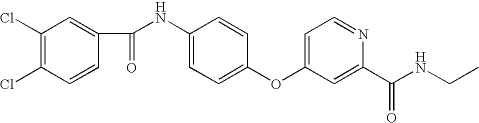 | 4-(4-(3,4-dichlorobenzamido)phenoxy)-N-ethylpicolinamide | 431 |
| 22 | 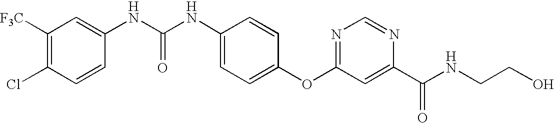 | 6-(4-(3-(4-chloro-3-(trifluoro-methyl)phenyl)ureido)phenoxy)-N-(2-hydroxyethyl)pyrimidine-4-carboxamide | 497 |
| 23 | 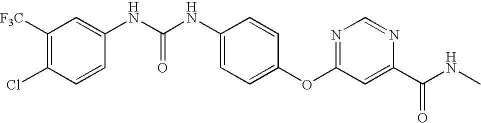 | 6-(4-(3-(4-chloro-3-(trifluoro-methyl)phenyl)ureido)phenoxy)-N-methylpyrimidine-4-carboxamide | 467 |
| 24 | 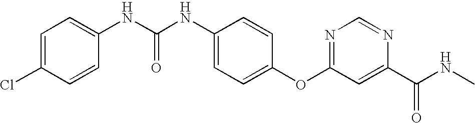 | 6-(4-(3-(4-chlorophenyl)ureido)phenoxy)-N-methylpyrimidine-4-carboxamide | 399 |
| 25 | 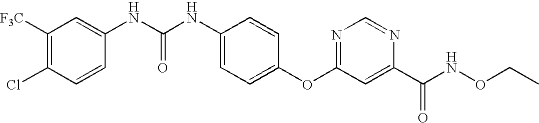 | 6-(4-(3-(4-chloro-3-(trifluoro-methyl)phenyl)ureido)phenoxy)-N-ethoxypyrimidine-4-carboxamide | 497 |
| 26 | 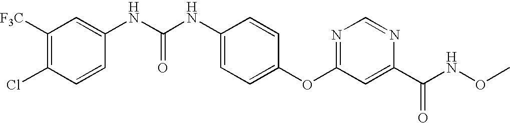 | 6-(4-(3-(4-chloro-3-(trifluoro-methyl)phenyl)ureido)phenoxy)-N-methoxypyrimidine-4-carboxamide | 483 |
| 27 | 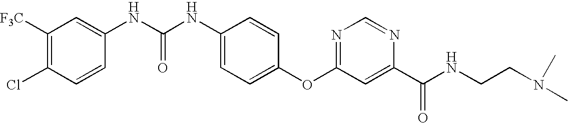 | 6-(4-(3-(4-chloro-3-(trifluoro-methyl)phenyl)ureido)phenoxy)-N-(2-(dimethylamino)ethyl)pyrimidine-4-carboxamide | 524 |
| 28 | 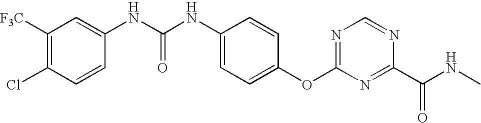 | 4-(4-(3-(4-chloro-3-(trifluoro-methyl)phenyl)ureido)phenoxy)-N-methyl-1,3,5-triazine-2-carboxamide | 468 |

### Example 6

This example demonstrated the assay of Raf inhibitors, which may be determined by the following in vitro Raf Kinase Assay:

Activity of human recombinant Raf protein was assessed in vitro by assay of the incorporation of radiolabelled phosphate to recombinant MAP kinase (MEK), a known physiologic substrate of Raf. Catalytically active human recombinant Raf protein was obtained by purification from sf9 insect cells infected with a human Raf recombinant baculovirus expression vector. To ensure that all substrate phosphorylation resulted from B-Raf activity, a catalytically inactive form of MEK was utilized. This protein was purified from bacterial cells expression mutant inactive MEK as a fusion protein with glutathione-S-transferase (GST-kdMEK).

Method: Standard assay conditions of Raf catalytic activity utilized 3 g of GST-kdMBK, 10 M ATP and 2 uCi ³³P-ATP, 50 mM MOPS, 0.1 mM EDTA, 0.1M sucrose, 10 mM MgCl₂ plus 0.1% dimethylsulphoxide (containing compound where appropriate) in a total reaction volume of 30 ul. Reactions were incubated at 25° C. for 90 minutes and reactions terminated by addition of EDTA to a final concentration of 50 uM. 10 ul of reaction was spotted to P30 phosphocellulose paper and air dried. Following four washes in ice cold 10% trichloroacetic acid, 0.5% phosphoric acid, papers were air dried prior to addition of liquid scintillant and measurement of radioactivity in a scintillation counter. The % inhibition of the B-raf kinase activity is calculated and plotted in order to determine the concentration of test compound required to inhibit 50% of the B-raf kinase activity (IC50).

Compounds 3, 4, 20, and 21 have an IC50 of less than 10 uM. Compounds 5-19, 22-28 have an IC50 of less than 1 uM.

We claim:

1. A compound represented as Formula (I) 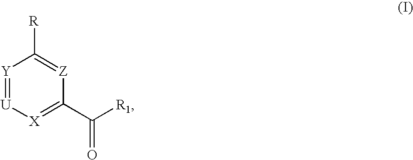
wherein
X represents N or CRₓ; Y represents N or CR_{y}; Z represents N or CR_{z}; U represents N or CRᵤ; wherein Rₓ, R_{y}, R_{z} and Rᵤ, independently represent hydrogen, halogen, C1-C4 alkyl, C1-C4 alkoxy, amino, or C1-C4 alkylamino; among U, X, Y and Z, at least one of them is N;
R represents Formula (II):
-L₁-Ar₁-L₂-Ar₂ (II),
wherein
L₁ and L₂ independently represent NR₂, NR₂CONR₂, NR₂CSR₂, NR₂CO, O, S, SO, SO₂ or CONR₃, or optionally represent cycloalkyl or heterocycloalkyl to link Ar₁ and Ar₂; wherein R₂ and R₃ independently represent hydrogen or C₁-C₄ alkyl;
Ar₁ and Ar₂ independently represent an aryl or heteroaryl, each of which is substituted with from 0 to 4 substituents independently chosen from:
(1) halogen, hydroxy, amino, cyano, —COOH, —SO₂NH₂, oxo, nitro or alkoxycarbonyl; or
(2) C₁-C₆ alkyl, C₁-C₆alkoxy, C₃-C₁₀ cycloalkyl, C₂-C₆ alkenyl, C₂-C₆ alkynyl, C₂-C₆ alkanoyl, C₁-C₆ haloalkyl, C₁-C₆ haloalkoxy, mono- or di-(C₁-C₆alkyl)amino, C₁-C₆ alkylsulfonyl, mono- or di-(C₁-C₆alkyl) sulfonamido, mono- or di-(C₁-C₆alkyl)aminocarbonyl, phenylC₀-C₄alkyl or (4- to 7-membered heterocycle)C₀-C₄alkyl, each of which is substituted with from 0 to 4 secondary substituents independently chosen from halogen, hydroxy, cyano, oxo, imino, C₁-C₄alkyl, C₁-C₄alkoxy or C₁-C₄haloalkyl;
R1 represents
1) OR₄
2) NR₄R₅, or
3) Formula (III) 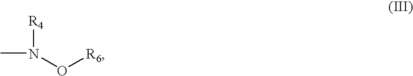
wherein R₄, R₅ and R₆ independently represent hydrogen, C₁-C₁₀ alkyl, C₁-C₁₀alkoxy, C₃-C₁₀ cycloalkyl, C₂-C₁₀ alkenyl, C₂-C₁₀ alkynyl, C₂-C₁₀ alkanoyl, C₁-C₁₀ haloalkyl, C₁-C₁₀ haloalkoxy, mono- or di-(C₁-C₁₀alkyl)amino, C₁-C₁₀ alkylsulfonyl, mono- or di-(C₁-C₁₀alkyl) sulfonamido, mono- or di-(C₁-C₆alkyl)aminocarbonyl, phenylC₀-C₆alkyl or (4- to 7-membered heterocycle)C₀-C₆alkyl, each of which is substituted with from 0 to 4 secondary substituents independently chosen from halogen, hydroxy, cyano, oxo, amino, imino, C₁-C₄alkyl, C₁-C₄alkoxy or C₁-C₄haloalkyl; and
only when X is N, L1 is O, Ar1 is phenyl and L2 is NHCONH, R1 represents Formula (III).

2. The compound according to claim 1, wherein one of U, X, Y and Z is N.

3. The compound according to claim 1, wherein two of U, X, Y and Z are N.

4. The compound according to claim 1, wherein three of U, X, Y and Z are N.

5. The compound according to claim 1, wherein X is N, R1 is Formula (III).

6. The compound according to claim 1, wherein Ar1 is optionally substituted phenyl.

7. The compound according to claim 1, wherein Ar2 is optionally substituted phenyl.

8. The compound according to claim 1, wherein Ar2 is substituted by up to 3 substituents independently selected from halo, trifluoromethyl, hydroxy, C₁₋₆alkyl, or C₁₋₆alkoxy.

9. The compound according to claim 1, wherein L1 is O.

10. The compound according to claim 1, wherein L1 is S.

11. The compound according to claim 1, wherein L1 is NH.

12. The compound according to claim 1, wherein L2 is O.

13. The compound according to claim 1, wherein L2 is S.

14. The compound according to claim 1, wherein L2 is SO.

15. The compound according to claim 1, wherein L2 is SO2.

16. The compound according to claim 1, wherein L2 is NHSO2

17. The compound according to claim 1, wherein L2 is CONH.

18. The compound according to claim 1, wherein L2 is NHCONH.

19. The compound according to claim 1, wherein L2 is NHCO.

20. The compound according to claim 1, wherein R1 is Formula (III):

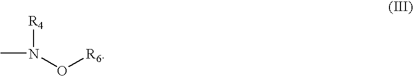

21. The compound according to claim 20, wherein R4 is hydrogen.

22. The compound according to claim 20, wherein R6 is methyl.

23. The compound according to claim 20, wherein R6 is ethyl.

24. The compound according to claim 20, wherein R6 is dimethylaminoethyl.

25. The compound according to claim 20, wherein R6 is diethylaminoethyl.

26. A compound selected from the followings:

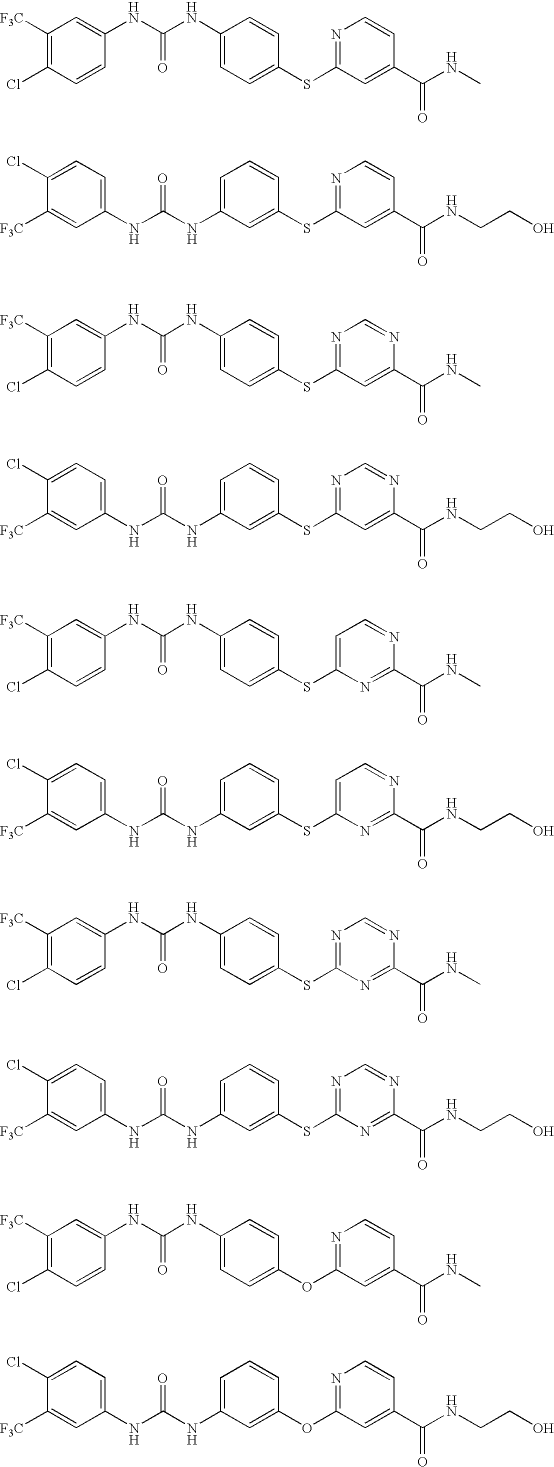

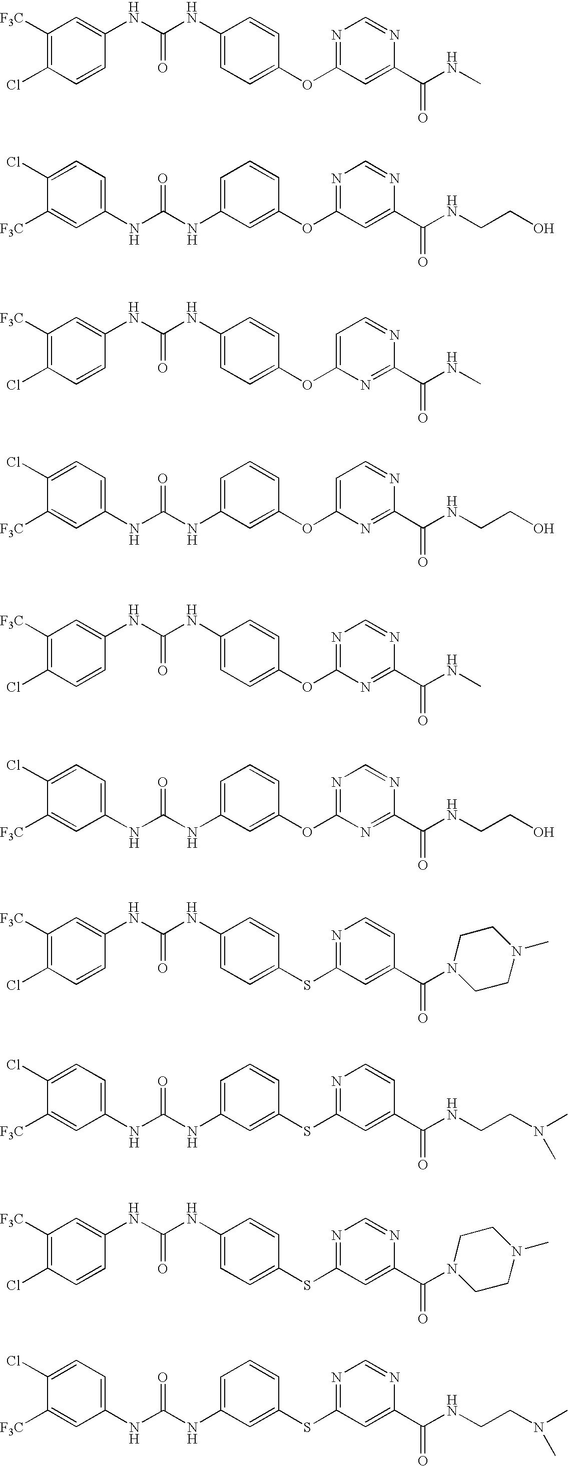

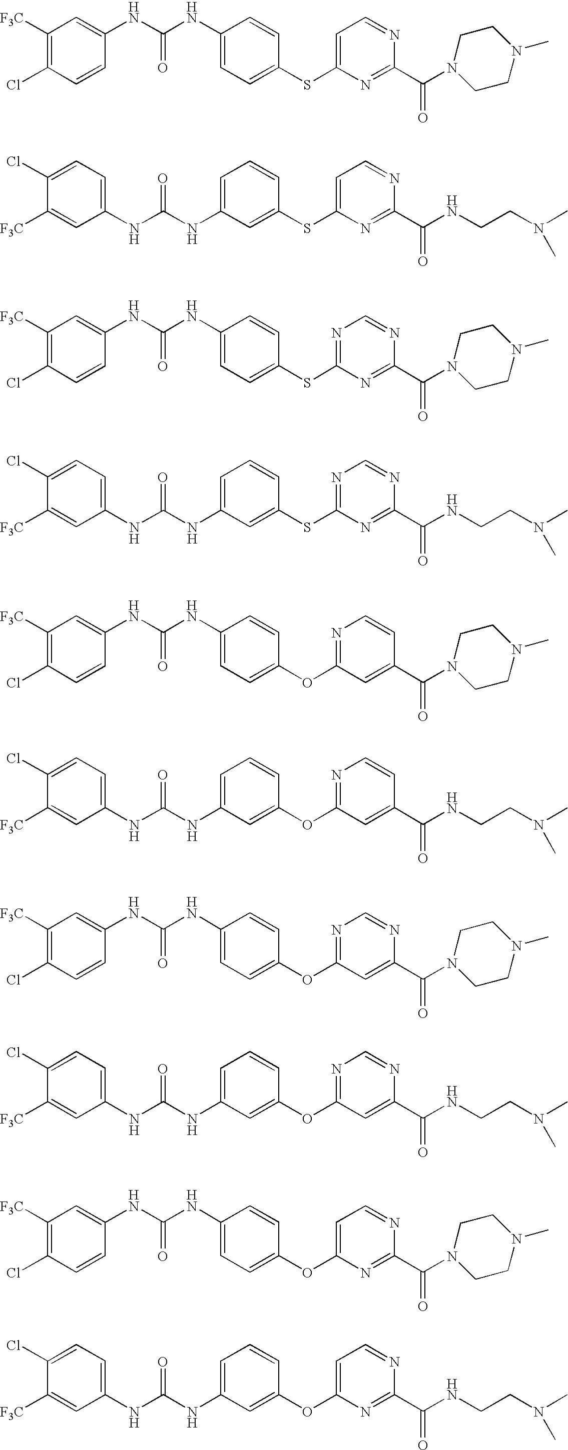

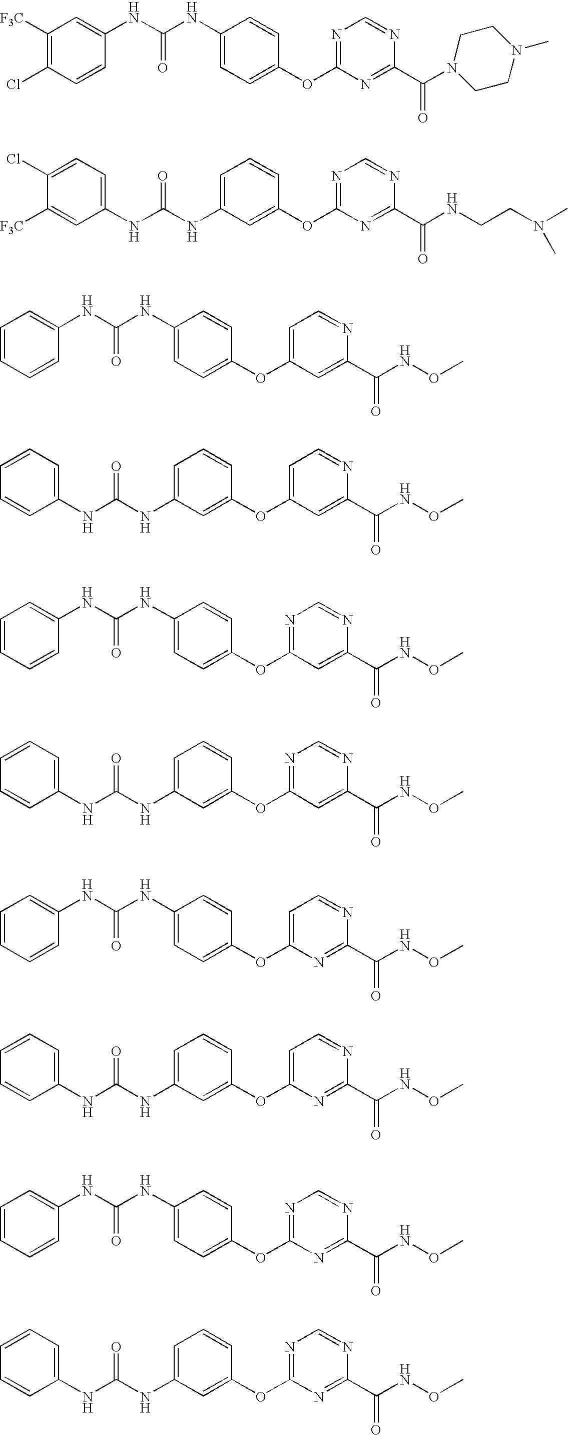

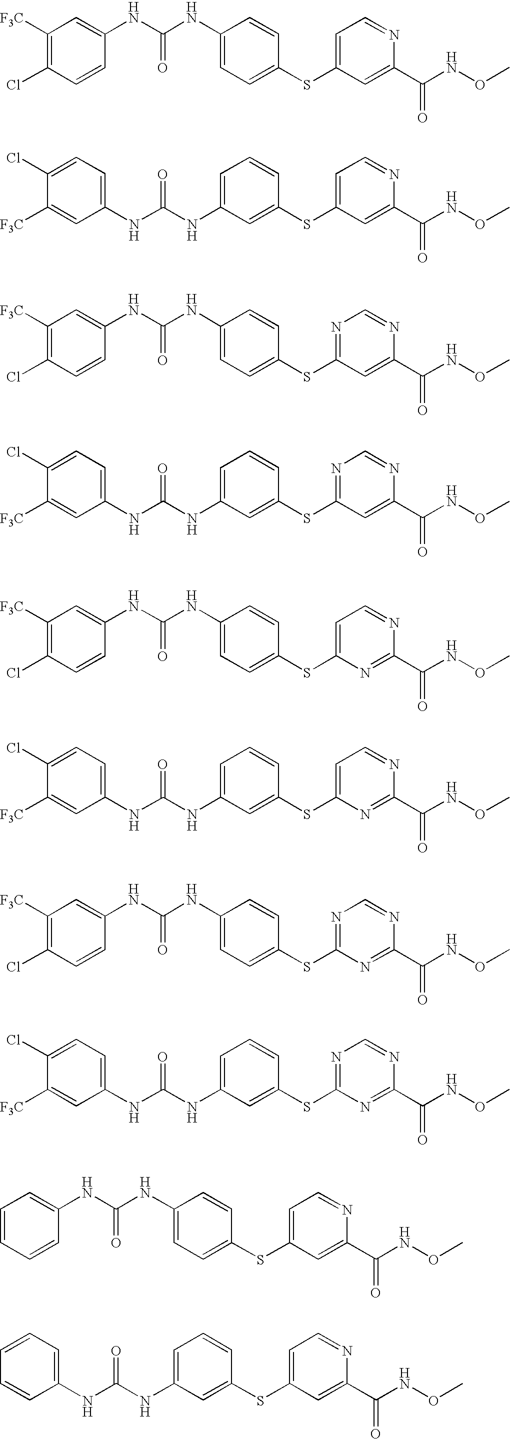

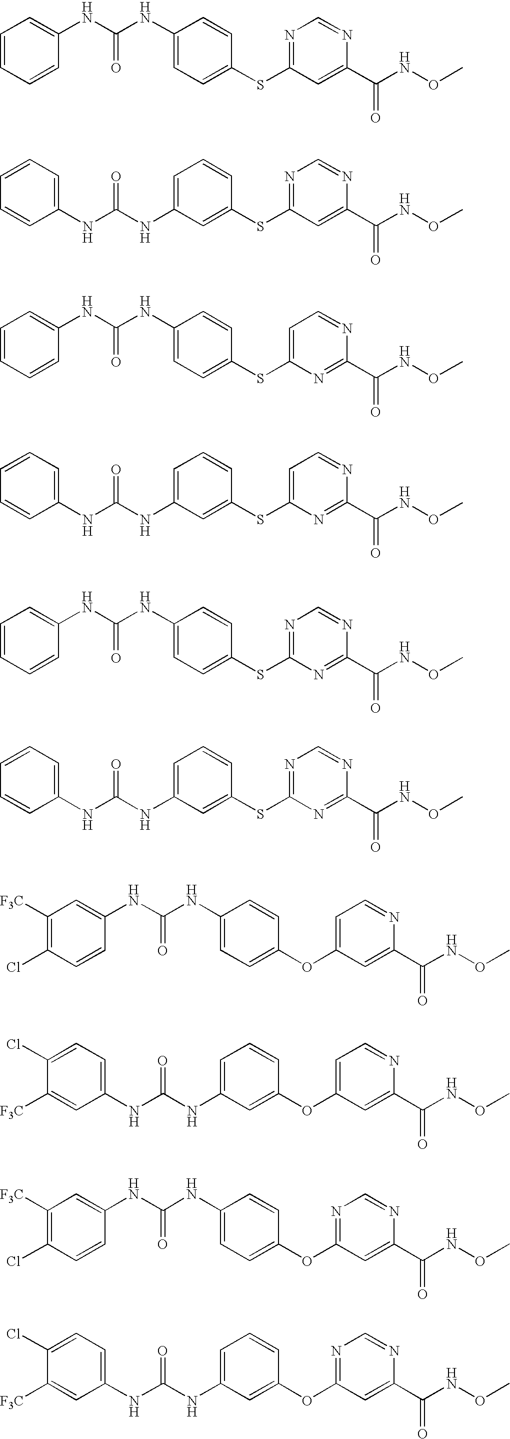

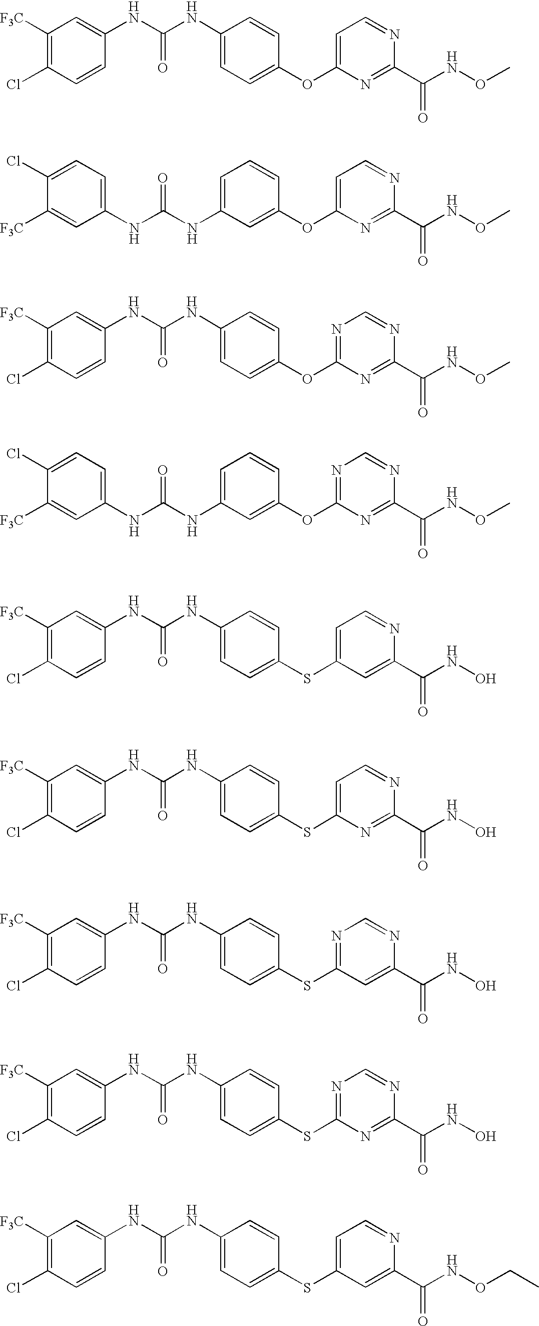

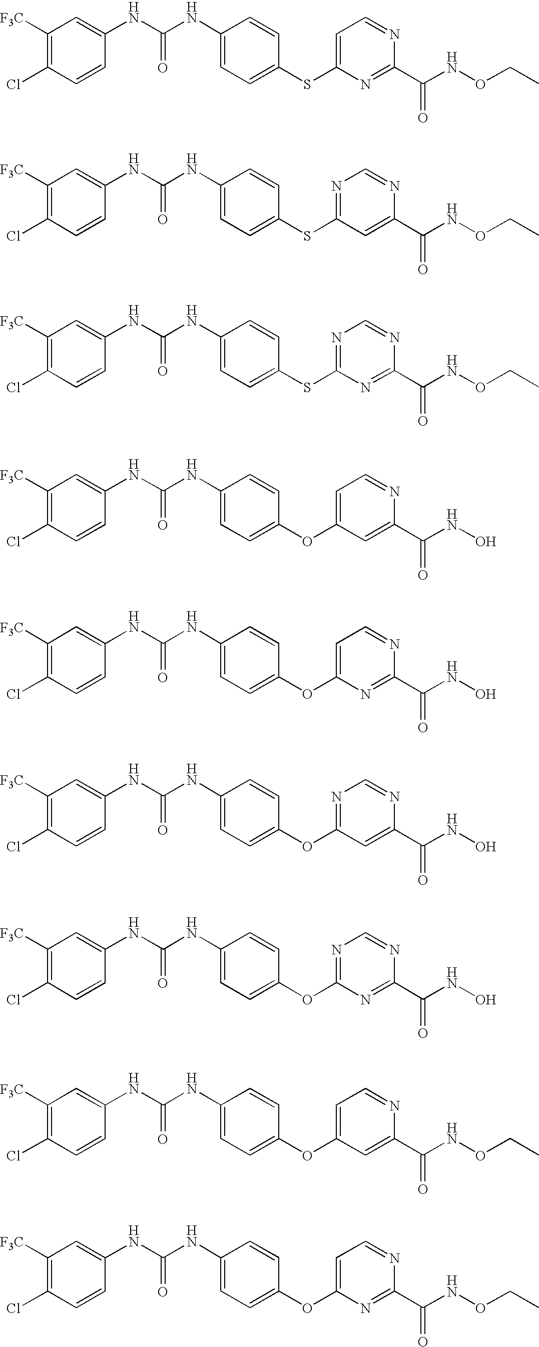

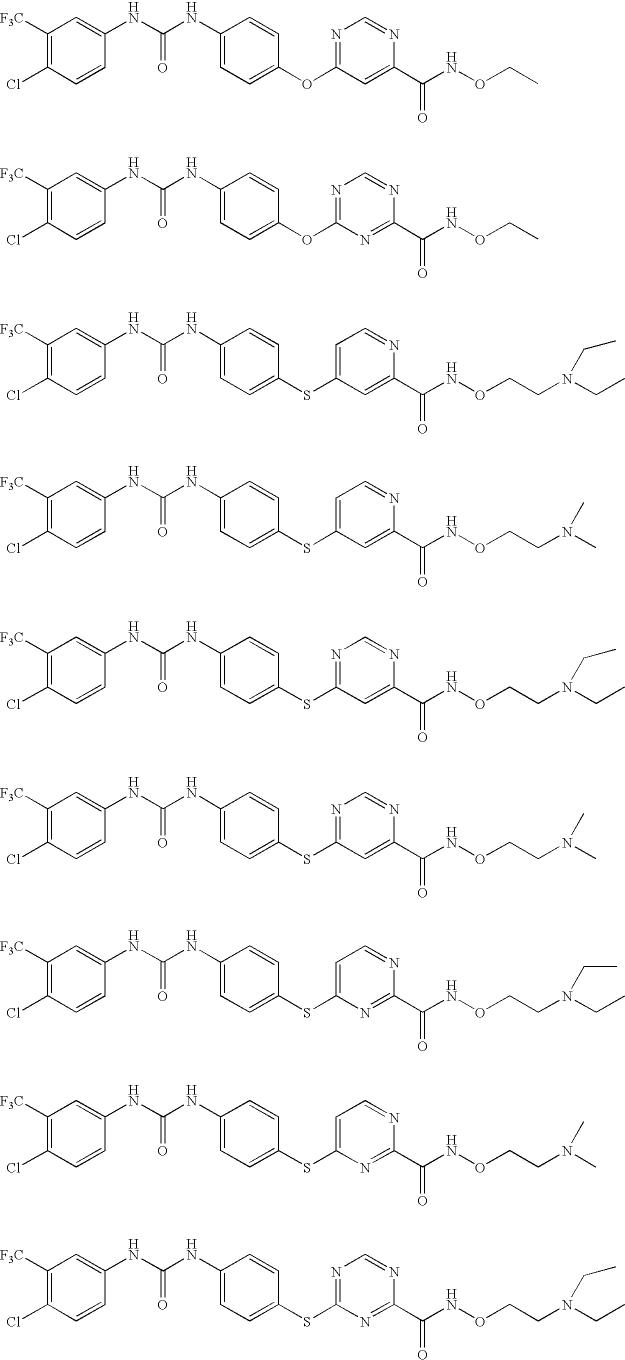

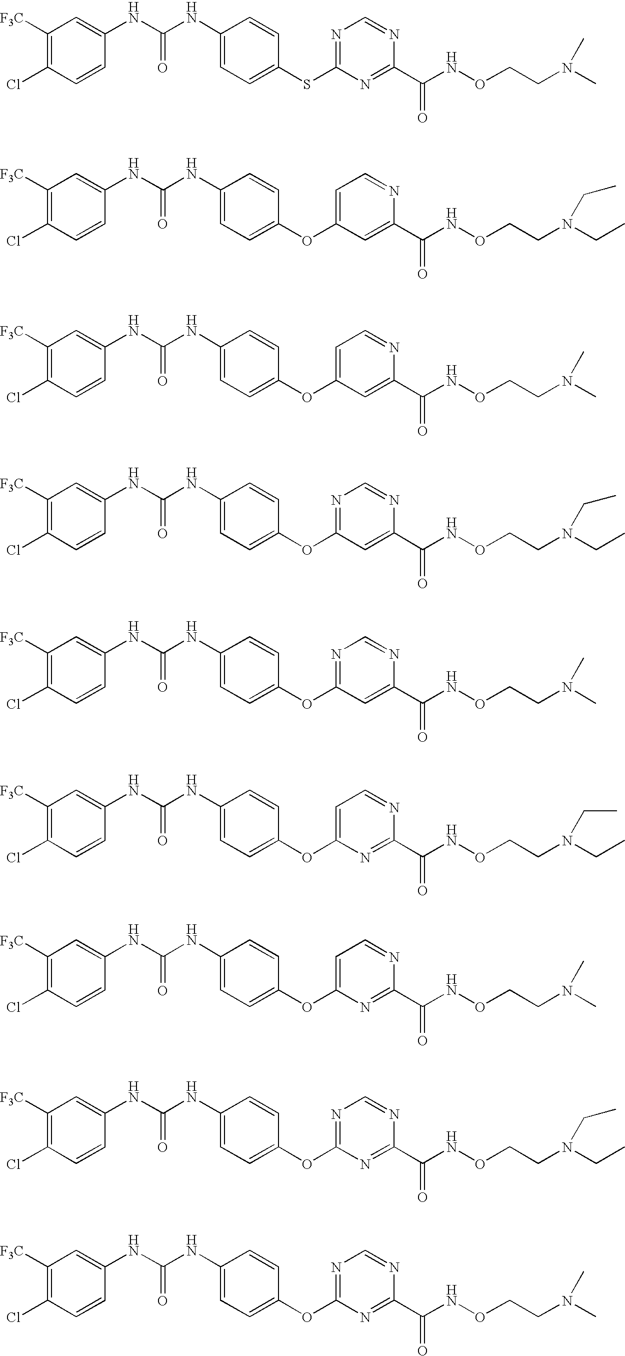

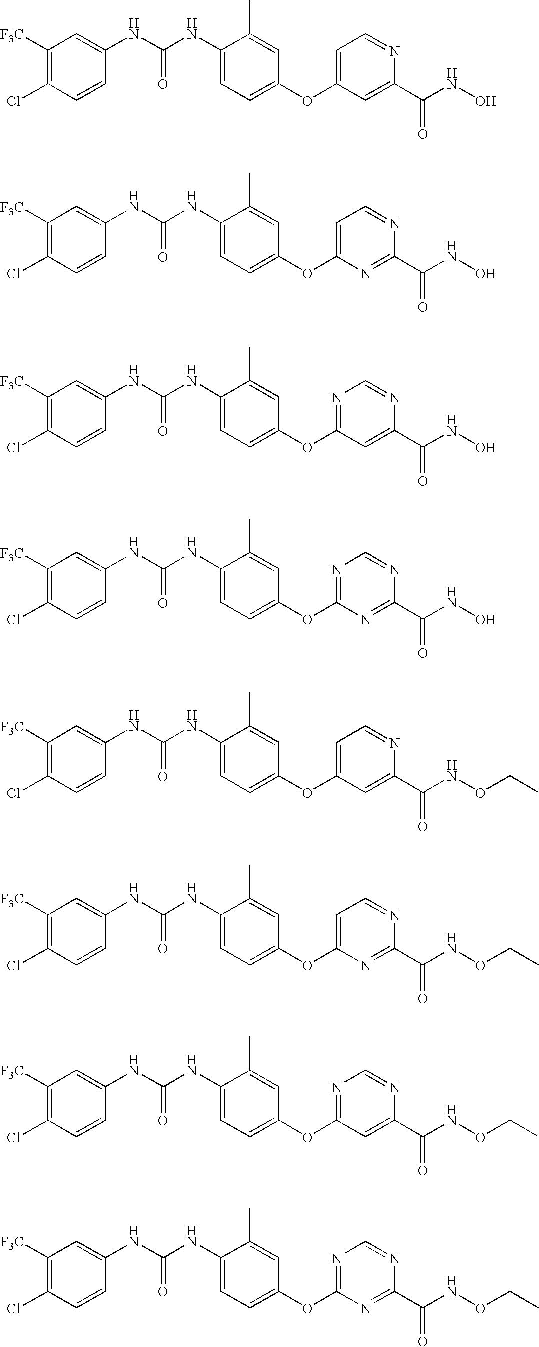

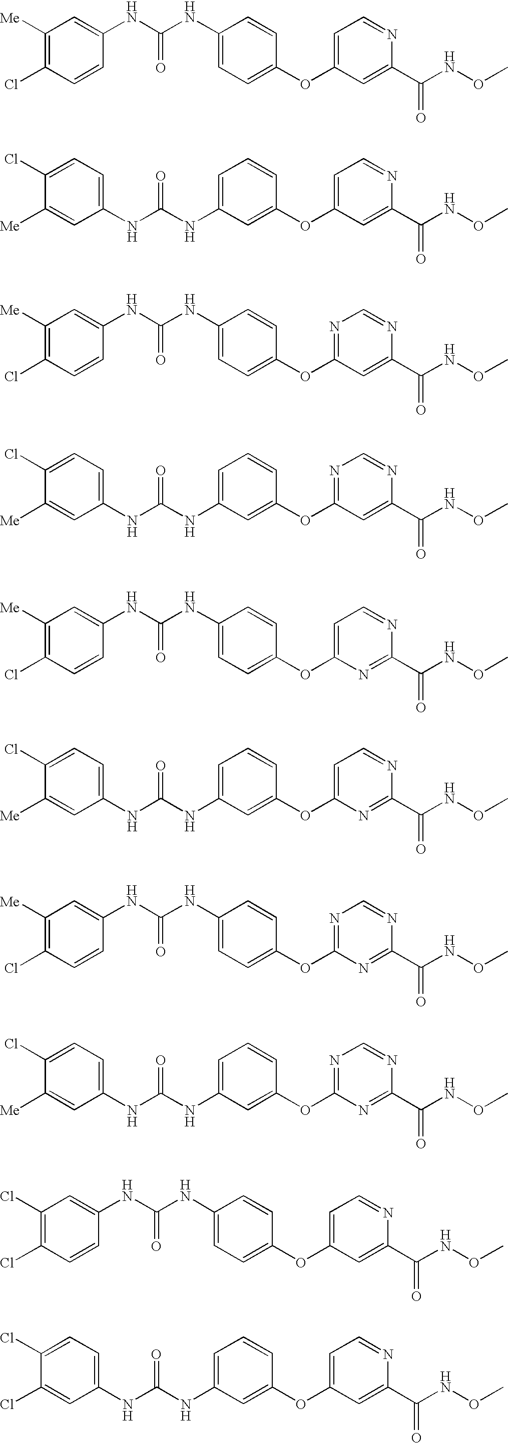

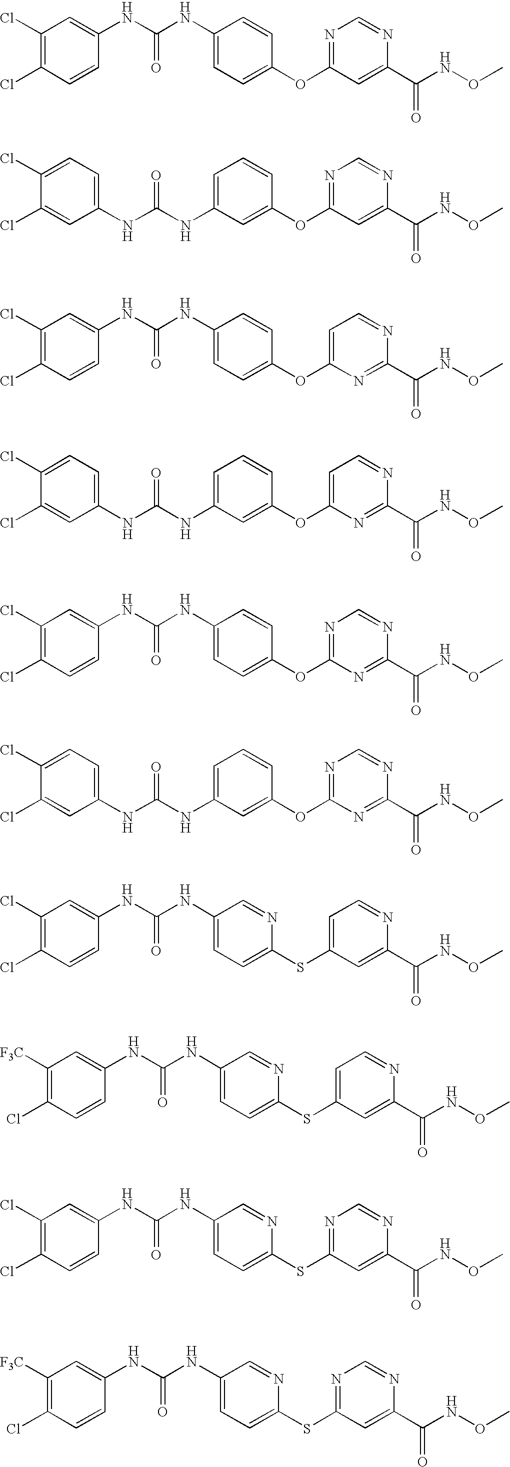

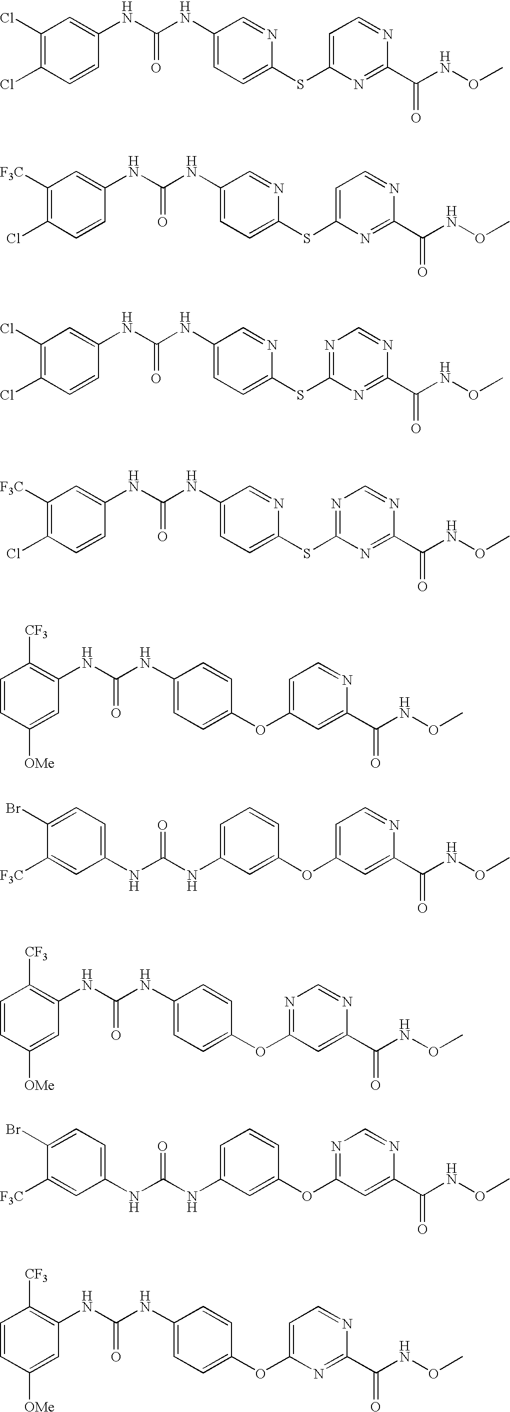

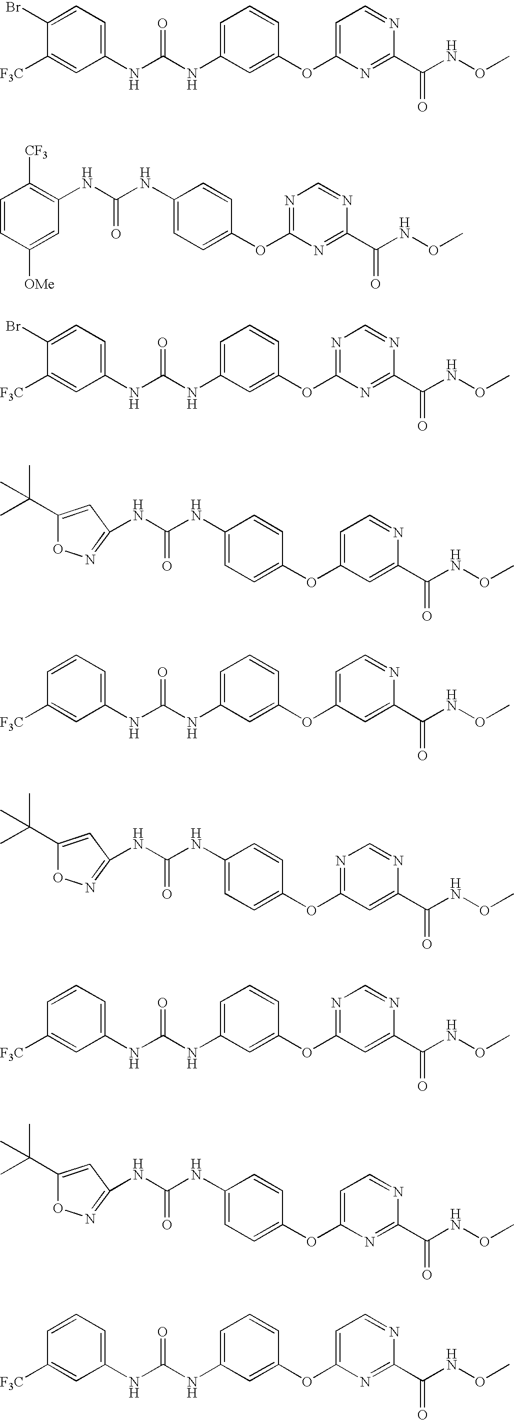

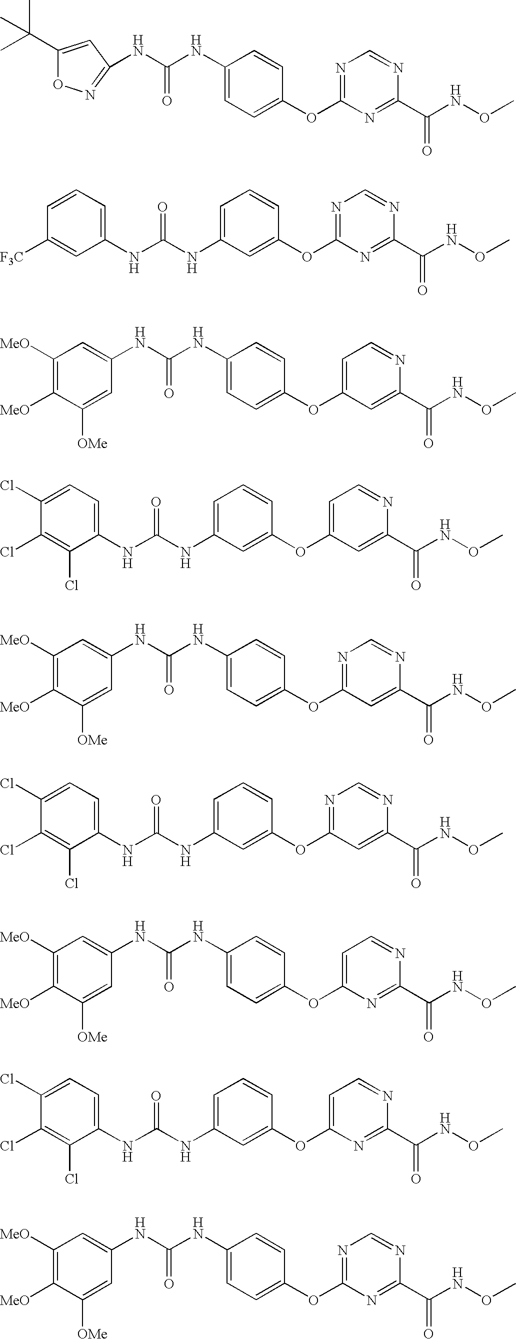

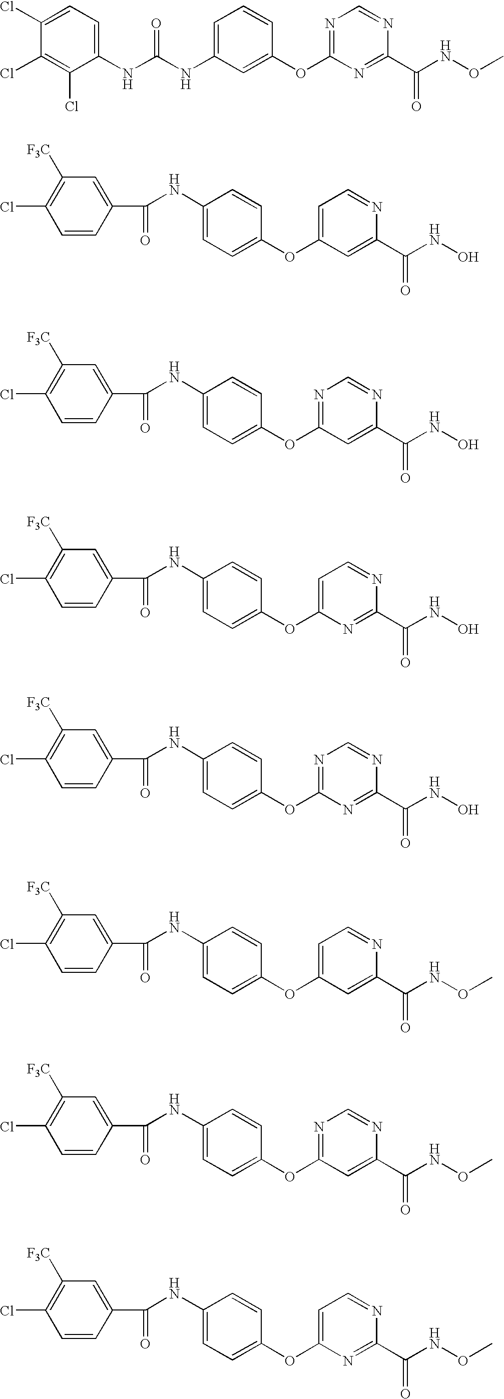

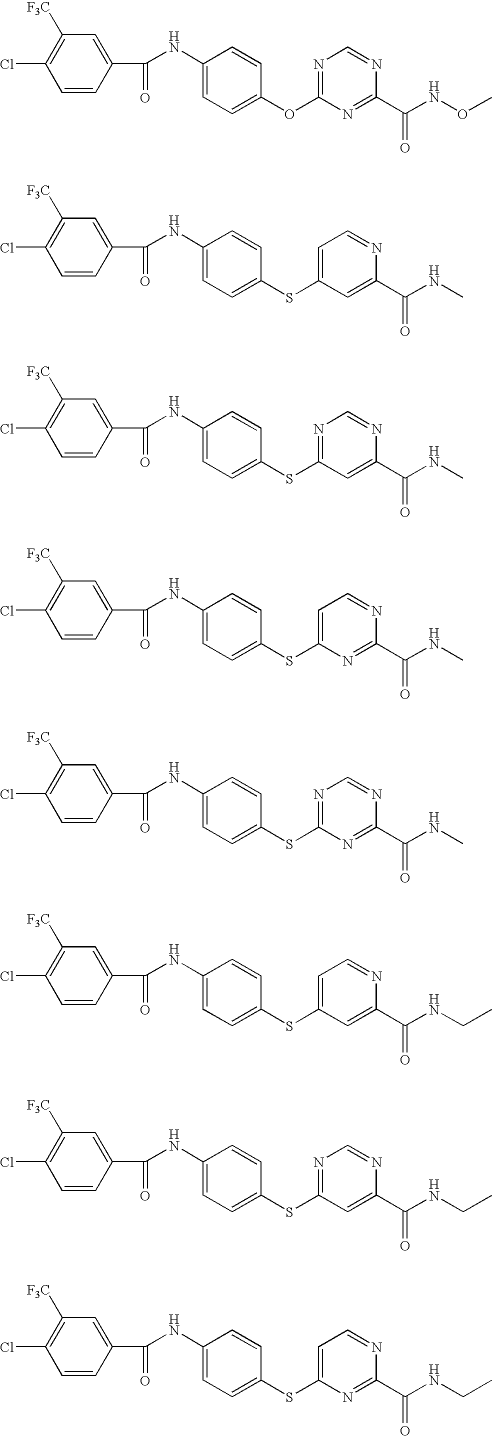

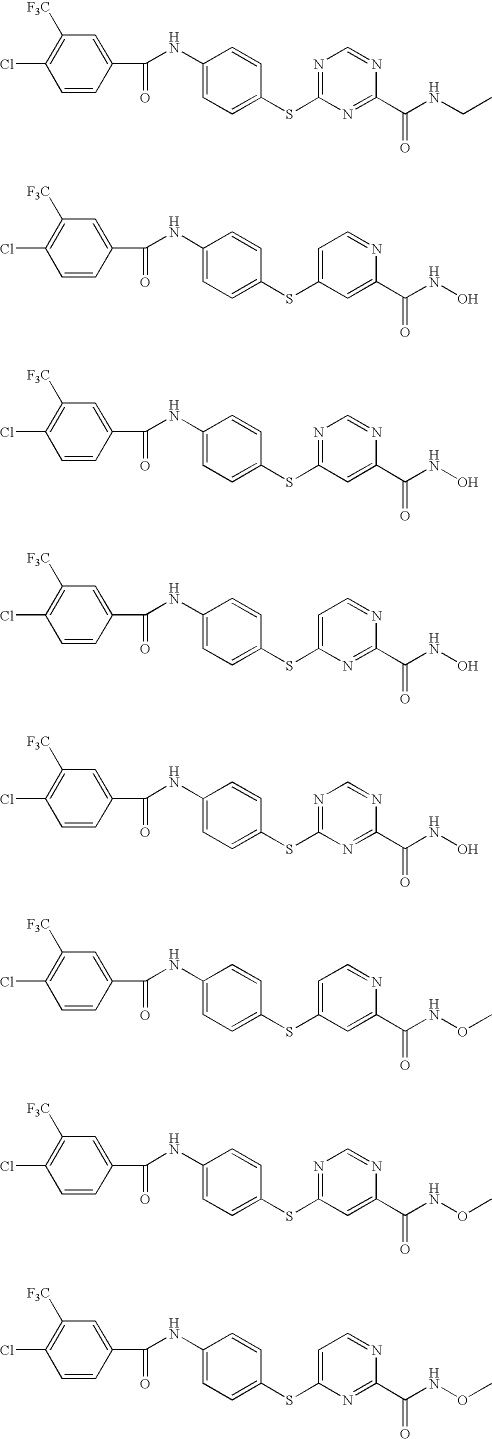

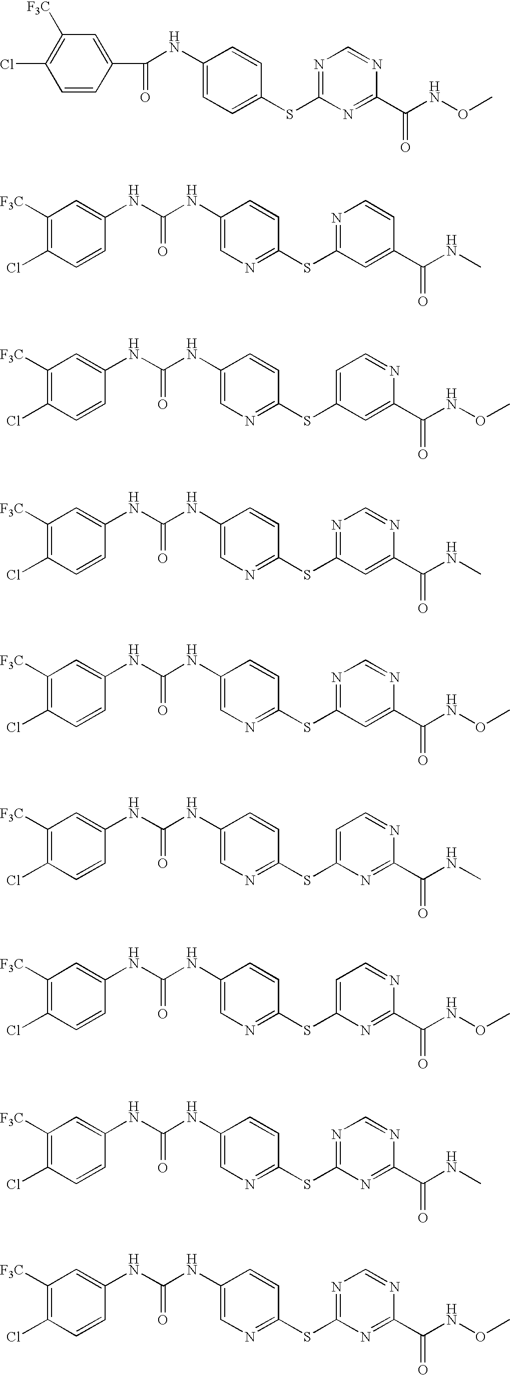

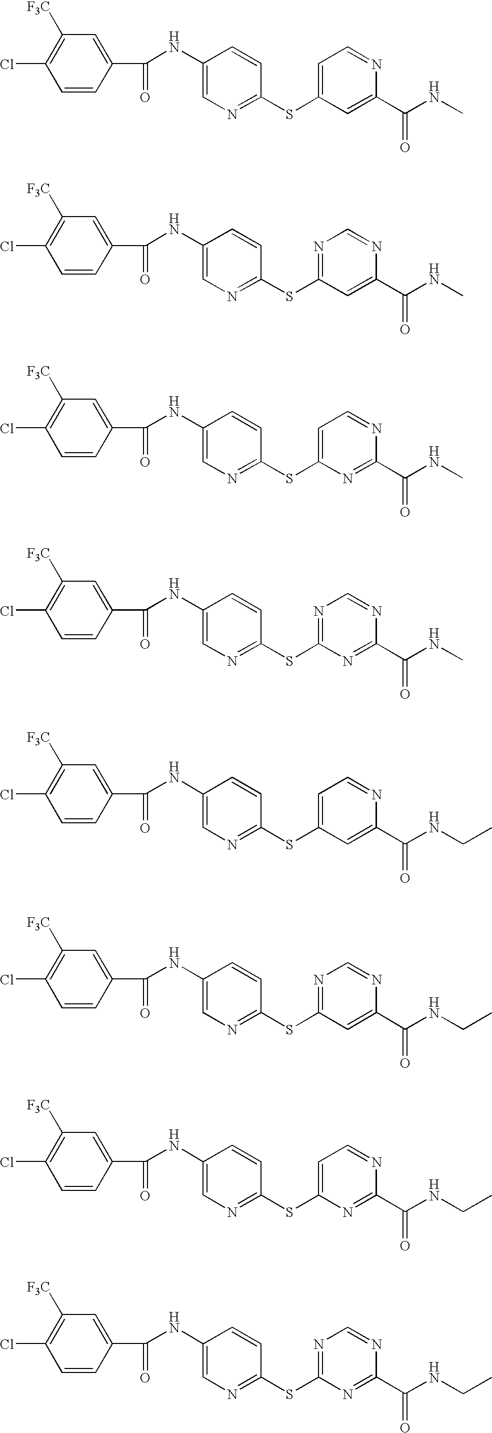

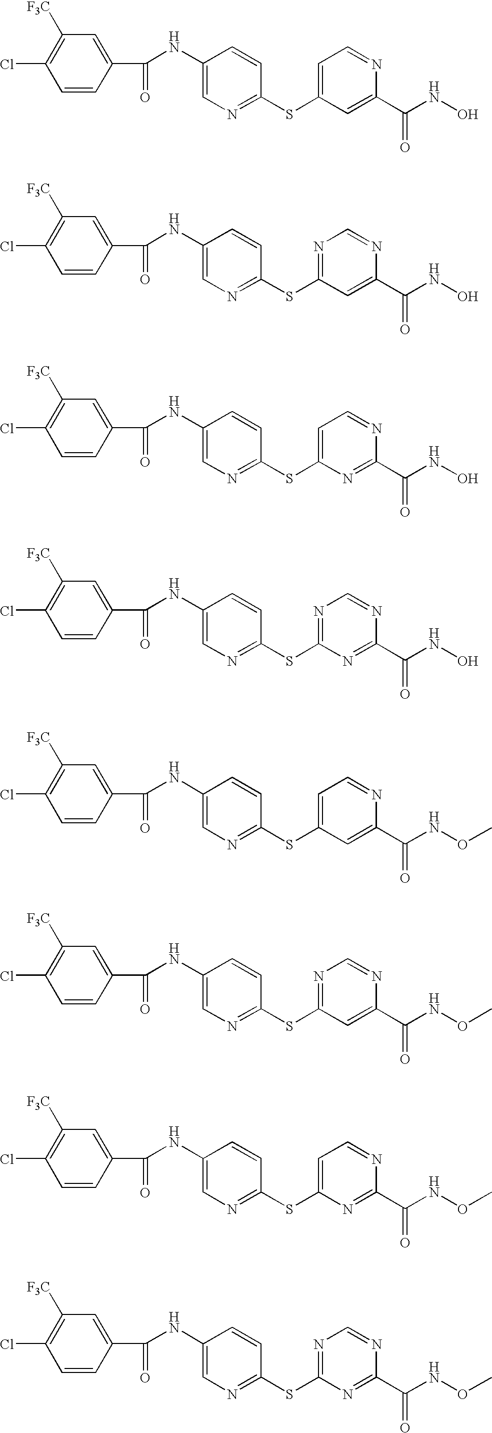

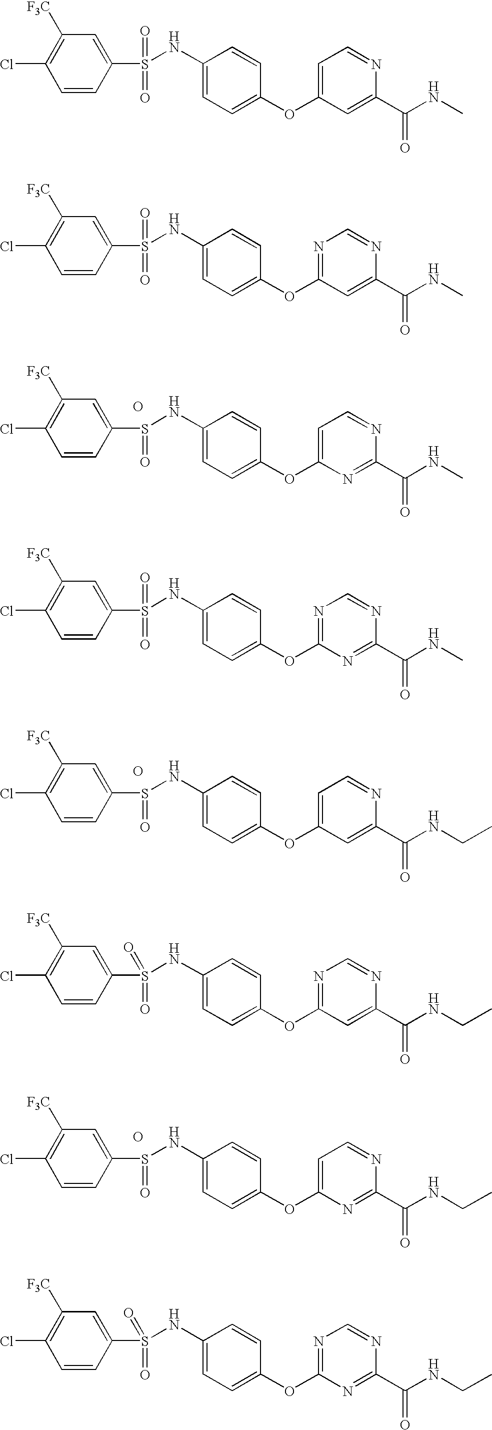

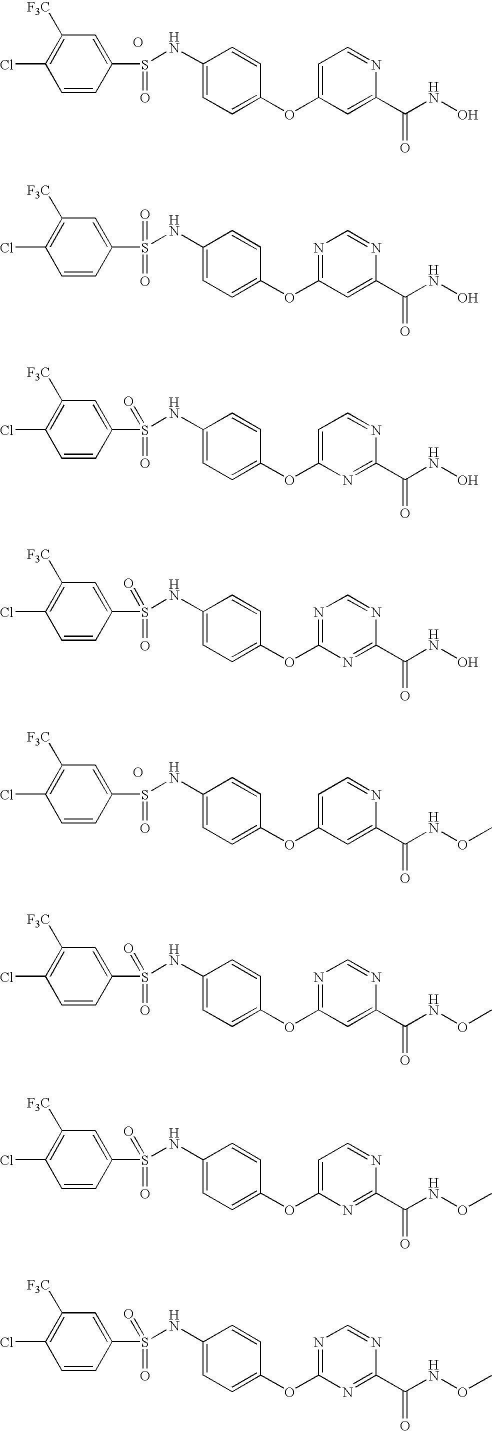

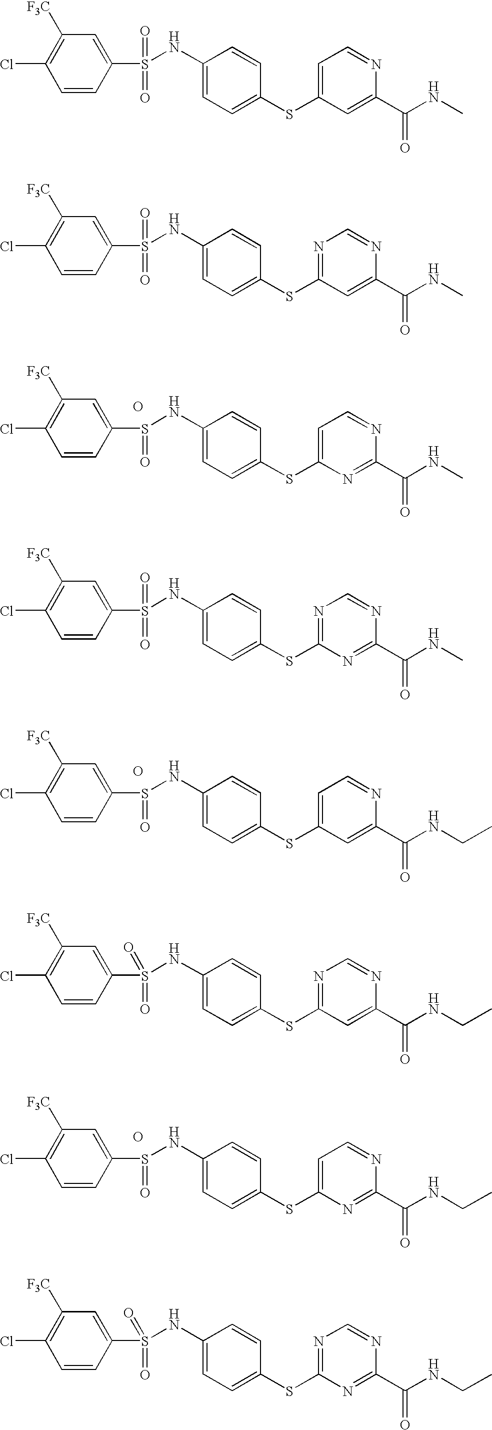

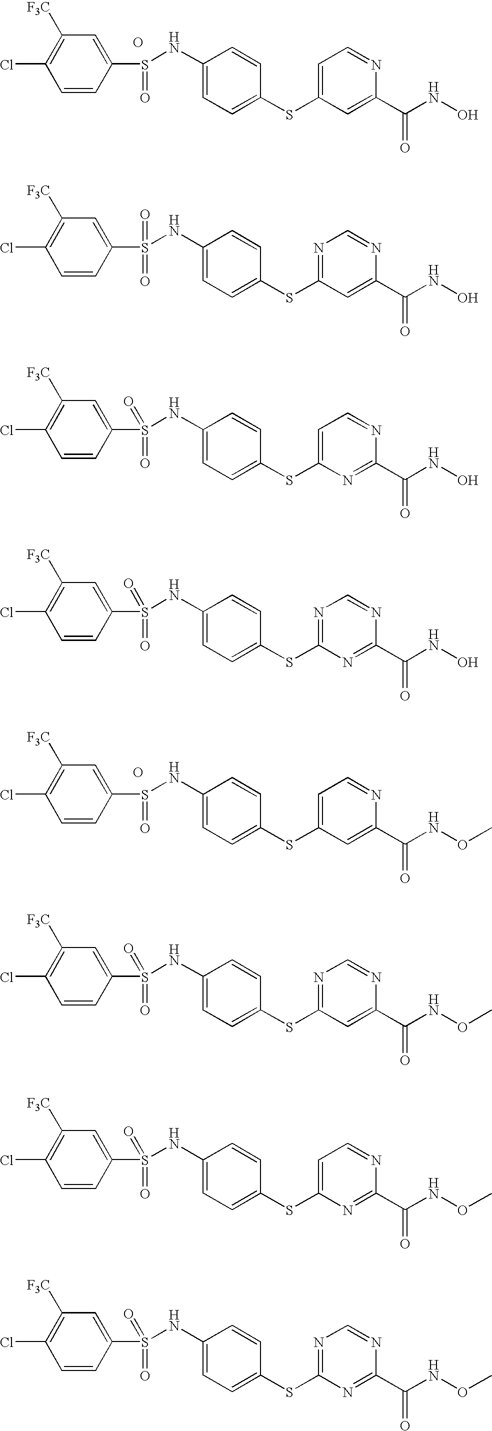

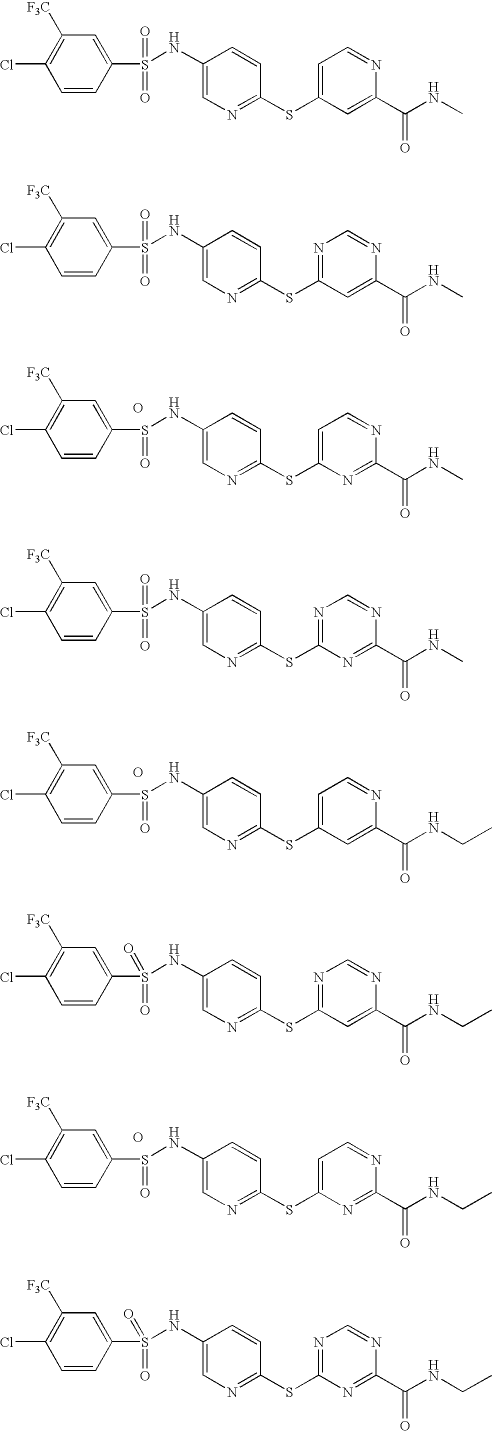

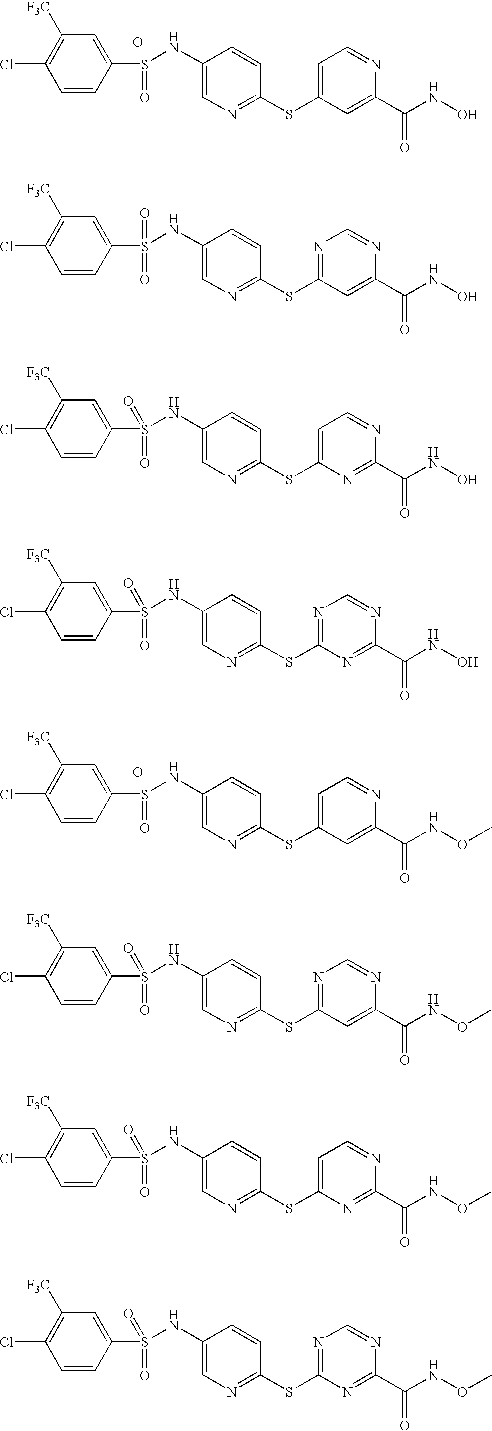

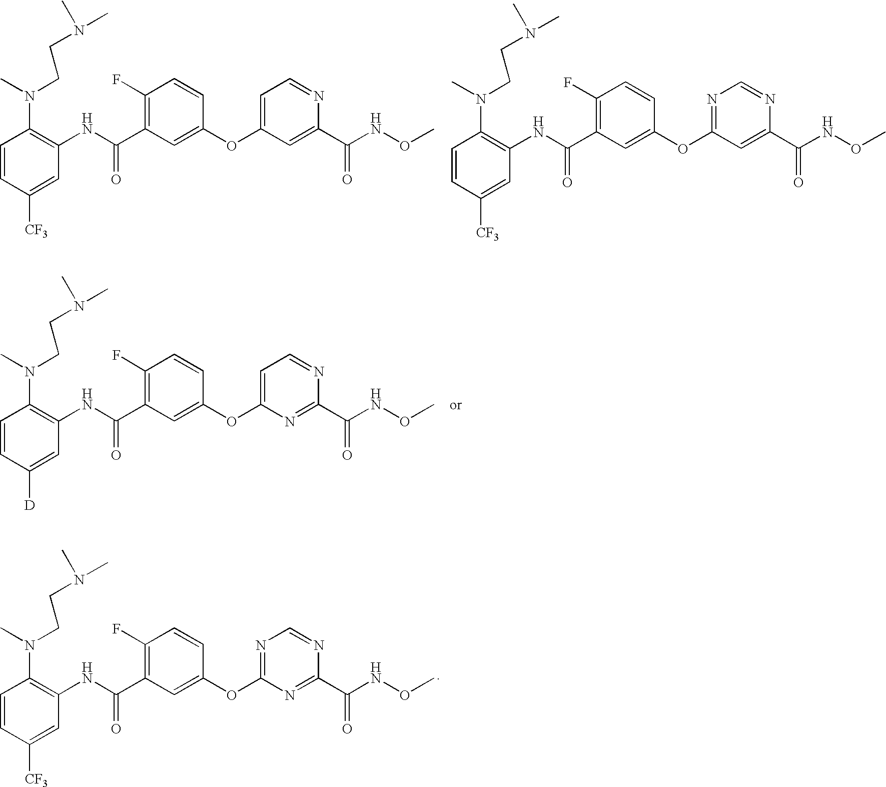

27. A pharmaceutical composition comprising the compound of claim 1 or pharmaceutically acceptable salts, hydrates, solvates, crystal forms salts or individual diastereomers thereof, and a pharmaceutically acceptable carrier.

28. The composition of claim 27, wherein the compound is present in an amount to detectably inhibit Raf protein kinase activity.

29. The pharmaceutical composition of claim 27, which is suitable for delivery via routes of administration selected from the group consisting of oral route, parenteral route, intravenous route, and combinations thereof.

30. A method for treating a disease or condition in a mammal characterized by undesired cellular proliferation or hyperproliferation comprising identifying the mammal afflicted with said disease or condition and administering to said afflicted mammal the composition of claim 27.

31. A method of treating or lessening the severity of a disease of condition selected from a proliferative disorder, a cardiac disorder, a neurodegenerative disorder, an autoimmune disorder, a condition associated with organ transplant, an inflammatory disorder, an immunologically mediated disorder, a viral disease, or a bone disorder, comprising a step of administering: a) the composition of claim 27; or b) the compound of any one of claims 1-26 to a patient.

32. The method of claim 31, wherein the disease or condition is cancer.

33. The method of claim 32, wherein said cancer is selected from the group consisting of cancers of the liver and biliary tree, intestinal cancers, colorectal cancer, ovarian cancer, small cell and non-small cell lung cancer, breast cancer, sarcomas, fibrosarcoma, malignant fibrous histiocytoma, embryonal rhabdomysocarcoma, neuro-fibrosarcoma, osteosarcoma, synovial sarcoma, liposarcoma, alveolar soft part sarcoma, neoplasms of the central nervous systems, brain cancer, lymphomas, and combinations thereof.

34. The method of claim 31, wherein the disease or condition is associated with a kinase.

35. The method of claim 34, wherein the kinase is a tyrosine kinase.

36. The method of claim 34, wherein the kinase is a serine kinase or a threonine kinase.

37. The method of claim 34, wherein the kinase is a Raf family kinase.

38. The method according to claim 31, comprising an additional step of administering to said patient an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an agent for treating liver disease, an anti-viral agent, an agent for treating blood disorders, an agent for treating diabetes, or an agent for treating immunodeficiency disorders, wherein said additional therapeutic agent is appropriate for the disease being treated; and said additional therapeutic agent is administered together with said composition as a single dosage form or separately from said composition as part of a multiple dosage form.
